SEQUENCE LISTING (3 sequences)

SEQ ID NO: 1 - DNA, 12436 bp, Homo sapiens
  source 1..12436: mol_type = other DNA
cccttctgtaggggcagagaggttctacttcattactgcgtctcctgggaaggccatcaggactgctggctaaagtgggaaccaggactctttgtgagttaagaatttgtgtatttatatgtgtgttatacacattttttaaaaaactgtaacgacatcaggttgagcagtcgtctccgggtggtgaattatgtgtatttttaaattttatactatattgttatttttcaaatgttcgaaattgaatatgtagattgttgttatcagcagaaaaataaacattattcaaatactctattcagtaaagtaatttattgggcgcctttgtcaagcacgcatttgcctagatgtgactctacagataaaattcacttggggcctccccttacagacaatcaggcagtggagactgagtgcctgaatggatagaccagcactcagaccactattttcagtatctgtttttcttaactcagggccgtggttttcaaacgtttttcgccttacggtcacccttagggtcccccgagaccggcccagacagacagatatacaaaaacacatacacagtcatgagcgtccaccatttccccaccaggcgcagcacaggcggcttcccggcactgagatgggggggaggagggagagagcgcgaggggggaggggaaagcagagaacgaaagaggcggaggcggcccccgaaccccgctctggtcttcatcatcaccacccctgggtccccagttcccacccacacaccaacctctaacgataccgggtaattttcctccttcttccctcaaacggctatagcgagacggtagacgacgaccagaactacttctgctcacgtaagcgagtaatcacgtgagcgcctacgtcatgtgagatctcggtcacgtgagcaactctcggcttaaactcgggatcactaaggtgccgcacttccttctggtatggaaatagggcgggtcaatatcaagaaaggaagagggtgattggttagcggaacgtcttacgtgactgattattggtctacctctggggataaccgtcccagttgccagagaaacaataacgtcattatttaataagtcatcggtgattggtccgcccctgaggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcaggtatcagatattgggtactcccttccctttgcttttccatgtgtttgggtgtgtttggggaactggagagtctcaacgggaacagttgagcccgagggagagctcccccacccgactctgctgctgcttttttatccccagcaaactgtcccgaatcaggactagccctaaactttctctgtgtgacctttcctgggatgggagtccggccagcggcccctgtttctttctctctctctctctctctcgttctccttctctttctctttctcttctttcctctctctttctctctctccctgcccggttctcttttttcactgctccttgcagagcagggccaccccataggcagtgtgcccaaagtagccctgcccggttctattcagacccttcttgtgaacttctgctcttcctctgccgggtgctaaccgttagaacatctagggtgggtaggaggaatggggaactaagattcgtgccattttttctccttttggggtcgtggatttctcggcagtatctcgagggagttagagagaccataaggtcgctgagatctctcccacctcgcccatgagcgtggcatcaggctggaaggttgacatggaggaactttatacatttacacctttgcgtgagggttgaggctggattagataggtattgaacatatctgaccctcacaatccttatctgtaaattgggattacaaccttttaatttcagggagctgacaaaaaaaatctgaaaaatagttcttatctcacacaggtgagttttcaaggagataacctatttaaagtacatagcacagcgcttgaccattcaactgcgcttacagagcaaatgttcaatgggaaaatgaatgtaaatctacaaatctgaatgaatatgtgtatttttctggagagaggatatttacctttcttcaaattctcaaagggctctgtgatttaaaaaaggttaggaatcactgatagatgttggtaaaaggtggcagtcacagtacatttctgtgtccataagttattcctatgaatatctttatagataaagtcaggatgttggtcagacatcacagaagaaattggccttgtaagtttcatgtgaccctgtggtacagtatgtgtggcaattttgcccatcacggatttttttttattggtatttgcatctgattataaaactaatgcatgatcattgcaaaaaatgtagataaagaagagcaaaatgaaaataaagatttccccccaccgttccaccacccagaaataatcatggtttaaatgttaatatacaaccttacaattgttttctatataaatgaaaacatagatttctttatttcattattttccataaaaaatggatcatgtttatgtcatgtttggctaatggcaagaccctggcacccagtctgggctcaaattctgcctcattgttacttagccctgtgacattgggtaaattacactttttttttttttttttttttgagacggggtctcgctctgtcgcccaggctggagtgcagtggcacgatctcggctcactgcaagtccgcctcctgggttcacgccattcttctgcctcagcctcccgagtagctgggactacaggcgcctgccaccacgcctggctctttttttttttttttttttttttagtacagacggggtttcaccatgttagccagggtggtctcaatctcctgacctcgtgattcgcccgcctcagcctcccaaagtgctggtgtgagccaccgtgcccagccttacttttttttttgagagggggtctcactctgtcacccaggttggagtgcagtggcgcgatctctgctcagtgcaaactccacctcccgggtttaagcagttctcctgtcgtagtctcctgagtagctgggattacaggcacaccaccacggccagctaatttttgtattttcagtagagacgggtttcaccatgttgcccaagctggtctcgaactcctggcctcaagtgatctgcccgccttggcctcccagagtgctgggattacaggtgtgagccaccgcacccggcctcttttttcttttttagtctatcataccttgcaaatacagtggttcttcctatgtgttggttttgatatttatgtaatcaaacacatcagtttttcctttctgatttctgactttggggtcatgctgagaaagtcctttcctacctgaagataatacagtatatacgtttcttactagtatttttgtggatttttaaaatatttaaatctttagtccatctgaacttgttcttctatcagaaatgccacatttaataaataataagtcccatggtatcagatggctggaaggacctctttcgaaactttgtttaattccattaatctgtgtattcttattctaatgctaatagttccacactagcttcctttatcttttttttctttttttttttttttttgagctggagtttcgctcttgttgcccaggctggagtacaatgtcacgatctcggttcaccgcaacctccgcctcccaggttcaagcaattctcctgcctcatcctcgcgagtagctggaattacaggcatgcgccaccacgcctagctattttgtatttttagtagagatggggtttctccatgttggtcaggctggtctcaaactcccagcctcaggtgatctgcctgcctcggcctcccaaaatgctgttattacaggcgtgagccaccacgcccagccttcatcttttaatgaatgtacatgtatgtaatcttttaggtgaactttttgtaatgttgtgccaagttccttaaaaagcccttttggaagctgggcaggtggccacgcctgtaatcccagcattttgggagtctgaggcaggtggatcacttgaggccaggagttcaagactagcctagccaaaatgcaaaaccctgtctctactaaagatacaaaaattagccggatgcgatggcacatgcctgtaatctcagctactcgggaggctgaggtagaagaatcgcttgaaccggggaggcagaggttgcagtgagcaagatggcgccactgcactccagcctgggtgacagagggagactccatctcaaaaaaaaaaaaaaaaaaaaagataaaaaggaaacctaagtactcttgggctttgttaaggattttgttaaatatacaaaggattgcagggaaaattaacttatttttaatattgagtatgcttatccaagagcaaaataatatttctccatttattcaaatcatttaggagcatcatagttttaacatatgggccttgcacgtatcttaaatttatctctaggcattttaggttgttcagttgttcttgtgaatgggatctttttctccaaataggattattgttgatatctgttgattatgttaactttgtagtttctgactttactgaactgtcttcttagatctaatactcttttcaatttcatcatatatttctcattcctattttgtttggggtttttagggcgggaatattaacgggataagagagacaaaagaaaatctggaaaaacaattcattttaccttacattgcttgtgattactaccacactattactgggttggaaaaaattgtgaaatcccaaggtgcctaataaatgggaggtacctaagtgttcatttaatgaattgtaatgattattggaatttctctttcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgtgagtttatagataatgttcttgttcattcagaggactgtaagcacttctgtacagaagcttgtttagaaacagccctcatggccgggcgtggtggctcacgctgtaatcccaacactttgggaggccgaggcgggtggatcacctgaggtcaagagttcaagaccagcctggccaacatggtgaaaccccaactctattaaaagtacaaaaaattagctgggcatggtggtgaacgcctgtaaccccagctacttgggaggctgaggcaggagaatcgcttgaacccaggaggtggaagtttcagtgagctgagatcacgccattgcactctagcctgggcaacaaaagagaaactccatctcaaaaaaaaaaacaaggaaaaaaagaaacagccctcatgacacttagaaagtagaatagctggctgttatctgaacattgaattgtaaggcttatcaggtggactttgcattccatcagcagacaatttttttttttttttttttttgagatggagtctcattctgtctcccaggctggagggcagtggtgcgatctcggctcactgcaagctccacctcctgggttcatgccattctcctgcctcagcctcccaagtagctgggaccacaggcacccgccaccatgcccagttaattttttgtatttttagtagagacggggtttcaccatgttagccaagatggtctcgatctcctgacctcgtgatccgcccacctcggcctcccaaagtgctgggattacaggcatgagccaccgcgcctagcctacaaatgttttgtaatagctcttgaggcccatcttggagttctccttttgctaaaaccactgaactctctaggaggaaaaaggaacttggttcttgacatatgtgtgcatgtatttccatataacctttaggaagctattgcaatggtactataaactagaattttagaagatagaaggaaaatattctggagatcattgaagagaaatggagtccaacactagttaaagatgatgaagacagatttttttttttgacggagtctcgctctgtcgcccaggctggagtgcagtggcacaatctcagctcactgcaaccctccacctcttgggttcaagtgattctcctgcctcagcctcccaagtagctgggactacaggcgcacaccaccacgcccggctaatttttgtatttttagtagagacaaggtttcaccatattcgccaggctggtctcgaactcctgaccttgtaatccgcccaccttggcctcccaaagtgctgggattacaggcatgagccaccacgcccggccgatgaagacagattttattcagtactaccacagtagaggaaagagccaagttcaattccaaatacaacaaagacaggtggagatttatagccaatgagcagattgagggggtcagtggatggaatatttaagaagacatcaagggtagggagcttcttgctaaagcttcatgtacttaaacaagaagggtgggggatgagggaaattgatcagatatcaatggtggcagtattgacttagcaggattcttgctaagaggtcttgctaggacagacataggaagccaaggtggaggtctagtcgaaaagaaggctcatcagagaagtctaactaaagtttggtcaagaagagtctttgtcaaggtaaatctatcatttccctcaaaaggtaattttcaggatcccatcaggaagattagcatggctgctagctttctcctcagttctgggctatagctcacatgcctagtttgaactagctcagcagaactgggggatttattctttgtcttccaacaaactcatctggatgattttgggggtttgtggggaaaagcccccaatacctggtgaagtaaccttgtctcttcccccagcctggaatggttctctctttctgctacctcacgattgtgcttctacaatggtgactcttttcctccctctcatttcaggttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggtaatgtttcattccagagatttagccacaaaggaaagaactttgaggccatggtagctgagccaaagaaccaatcttcagaattttaaataccctgtcacaatactggaaataattattctccatgtgccagagctcccatctcttctctttcagttcattaattaattaattaattcatgtaaaatccatgcatacctaaccatagctaatattgtgcacttataattcaagagggctctaagagttaattagtaattgtaactctctataacatcatttaggggagtccaggttgtcaatcggtcacagagaaagaagcatcttcattcctgcctttcctcaatatacacaccatctctgcactacttcctcagaacaatcccagcagtctgggaggtactttacacaatttaagcacagagcaactgcctgtccctgctgctagtttaaacatgaaccttccaggtagcctcttcttaaaatatacagccccagctgggcatgatggctcatgcctgtaatcctagcactttgggaggctgaggcgggtggattacttgaggtcaggagttcgagaccaccctggccaacatggtgaaaccccatctctagtaaaaatacaaaaattagctgactttggtggcacatgcctgtaatcccagctacttgggaagctgagacagaagagtcacttgaacctgggaaacagaggttgcagtgagccaagatcgcaccactgcactccaccctggatgacagactgaaccccatctcaaaaaattaaaataaaataaaataaaataactatatatatagccccagctggaaattcatttctttcccttattttacccattgttttctcatacaggttataagcacatgtccttggccctgaataggactggcagaagcattgtgtactcctgtgagtggcctctttatatgtggccctttcaaaaggtgagatagtgagcccagaatccaatagaactgtactgatagatagaacttgacaacaaaggaaaccaaggtctccttcaaagtccaacgttacttactatcatcctaccatctctcccaggttccaaccacttctcaccatccccactgctgtaattatagcctaagctaccatcacctggaaagtcatccttgtgtcttcccctttatttcaccattcatgtcctgtctatcaacagtccttccaccagtatctctaaaatatctcctgaatcagcccacttccttccatcttcactacatgcaccctggccttccaagctactatcggctctcaaccagactgctgggaccacctgatctctctgcttccactctgtctcaacccccatctattttccaagcagcactagagttatcatattaaaatgtaaatatcagttttttttttaaagaaaaaaaccctgagacttaacagagttataaaaaatataaatgtcatcatcagttccctgcttaaaacccttaactcgcttccaattgcacttggaatgaaaccaaactgcactgatccagcccttgcctgcctccccaaagtccaaggggtcatggctctttccctggctacactggttttctttctgtccctcaacactgcaagcctattgctgccccagggcctttacacttgctttttttctgcctagaacagttcttccccaaagatttttaaagggccgggctccttaacattgaagtcgcagaccaaacgccacatatgcagacagttcttctctaactactttaaaatagccctctgtccattcattcttcatcacattaacctgtttaattttcttctcagagctccacactatttggaagtatttgttgacttgttaccatgtctccccactagagtgtaagtttcatgagggcagggaccttgtctgactttgactgtatctctcgcatatggttaagtgttaaatagttatttatggaatgaatccctattattccctcattatctctgcaaaatagtcttttttctcaacatcttaaacctgatatcccacctgcctatctacaaactttttttttgcgacagagtctcactgtcacccaggctagagtgcagtggcgccatctcggctcactgcaacctccgcctcccgggtttaagcgattctcttgcctcagcctcccagtagctgggattataggcgtgcgctaccacatctggctaatttttgtatttttagtagagatggtttcaccatgttggccaggcttgtctcgaactcctgacctcagatgatccacctgcctcggcctcccaaagtgctgggattacaggcatgagccaccgtgcccagcctctacaaactttttattccattaacaaactatatgctgggatttaagttttcttaatacttgatggagtcctatgtaattttcgagcttttaattttactaagaccattttagttctgattatagaagtaaattaactttaagggatttcaagttatatggcctacttctgaagcaaacttcttacagtgaaaattcattataagggtttagacctccttatggagacgttcaatctgtaaactcaagagaaggctacaagtgcctcctttaaactgttttcatctcacaaggatgttagtagaaagtaaacagaagagtcatatctgttttcacagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatggtaaaaacttgagccctccttgttcaagaccctgcggtaggcttgtttcctattttgacattcaaggtaaatacaggtaaagttcctgggaggaggctttatgtgagagtacttagagcaggatgctgtggaaagtggtttctccatatgggtcatctaggtaactttaagaatgtttcctcctctcttgtttgaattatttcattctttttctcagttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggtaaataagagtatatattttaagatggctttatatacccaataccaactttgtcttgggcctaaatctatttttttcccttgctcttgatgttactatcagtaataaagcttcttgctagaaacattactttatttccaaaataatgctacaggatcattttaatttttcctacaagtgcttgatagttctgacattaagaatgaatgccaaactaacagggccacttatcactagttgctaagcaaccacactttcttggtttttcagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtttattttattgccaactactacttcctgtccacctttttctccattcactttaaaagctcaaggctaggtggctcatgcctgtaatcccagcactttgggaggctgaggcgggcagatcacctgaggtcgggactttgagacccgcctggacaacatggtgaaaccccatttctaataaaaatataaaaattagccaggtgtggtggcgcacctgtggtcccagctactctgggggctgaggcatgagaatcgcttgaacccgggagtggaggttgcattgagctgagatcatgccacctcactccagcctgggcaacaaagattccatctcaaaaaaaaaaaaaaagccaggcacagtggctcatgcctggaatcccagcacttttggaagctgaggcaggcagatcacttgaggttaggatttcaagaccagcctggctaacatagtaaagccctgtctctactaaaaatacaaaaattagccaggtatggtggcgagcttctgtagccccagctactcaggagactgaggcaggagaatcacttgaacccgggaagtgggggggtgcagtgacccaagatcacgccactgcattccagcctgggcaacagagcaagactccatctcaaaaaaaaaagttctatttccttgaataaaattttccgaagtttaaactttaggaataaaactattaaacccgtatttactcatccagatacccaccccccttgttgagattctctcccaattatcaaaatgtgtagcatatttaactaccaagagctaaacatcattaagactgaaatgtattaagaaggatgtataggccaggcacggtgtctcacgcctgtaatcccaacactttgggaggccaagtcgggcggatcacgaggtcaggagatggagaccatcctggccaacatggtgaaaccccctctctactaaaaatacaaaaattagccaggcaggtggcaggcacctgtaatcccagctactccagaggctgaggcaggacaatcacttgaacctgggaggcagaggctgcagtgagctgaggttgtaccaattgcactccagcctaggtaacgagcaacactccatctcaaaaaaagaaaaaaaaaaagatgtataatttggaactgttaagaggcattttaaaga

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 3 - DNA, 1290 bp, Homo sapiens
  source 1..1290: mol_type = other DNA
atgcagctgaggaatcccgagctccacctgggctgtgctctggctctgcggttcctggccctcgtgtcctgggacatccctggcgctagggccctcgataacggactggcccggacccccacaatgggatggctccactgggaaaggttcatgtgcaatctggactgtcaggaggaacccgactcctgcatcagcgaaaagctcttcatggagatggccgagctgatggtgagcgagggctggaaggacgccggctacgagtatctgtgcatcgatgactgctggatggcccctcaaagggactccgaaggcaggctgcaggctgatccccaaaggtttccccacggaatccggcagctcgccaactacgtgcattccaagggcctcaagctcggcatctacgccgacgtgggcaacaaaacatgcgccggattccccggcagcttcggctactacgacatcgacgcccagacattcgctgattggggagtggacctgctgaagttcgacggctgttactgcgattccctggaaaacctggccgacggctacaaacacatgtccctcgccctgaaccggacaggcaggtccatcgtgtacagctgcgagtggcccctgtacatgtggcctttccagaagcccaactacacagagatcaggcagtactgcaaccactggaggaacttcgctgacatcgacgactcctggaagagcatcaagagcatcctggactggaccagcttcaaccaggagaggatcgtggacgtggctggacccggaggctggaacgaccccgatatgctggtgattggcaacttcggactgagctggaaccagcaggtgacccagatggccctgtgggccattatggccgctcccctgttcatgtccaacgacctgaggcacatcagcccccaggccaaggctctgctgcaggacaaggatgtgatcgccatcaaccaggaccccctgggcaagcagggctaccagctgaggcaaggagataacttcgaggtgtgggagaggcccctgtccggactggcttgggccgtggccatgatcaatcggcaggagatcggcggaccccggtcctacaccattgctgtggccagcctgggaaaaggagtcgcctgcaaccccgcctgcttcattacccagctgctccccgtgaagcggaagctgggcttctatgagtggaccagcaggctgaggtcccatatcaatcctaccggcaccgtcctcctccagctcgagaataccatgcagatgagcctcaaggatctgctgtga

(19) United States
(12) Patent Application Publication
(10) Pub. No.: US 2026/0076951 A1
(43) Pub. Date: Mar. 19, 2026

(54) METHODS OF TREATING FABRY DISEASE IN PATIENTS HAVING RENAL IMPAIRMENT

(71) Applicant: Amicus Therapeutics, Inc., Philadelphia, PA (US)

(72) Inventor: Franklin Johnson, Bridgewater, NJ (US)

(73) Assignee: Amicus Therapeutics, Inc., Philadelphia, PA (US)

(21) Appl. No.: 19/333,835

(22) Filed: Sep. 19, 2025

Related U.S. Application Data
(60) Provisional application No. 63/696,627, filed on Sep. 19, 2024.

(51) Int. Cl. A61K 31/445; A61P 13/12

(52) U.S. Cl. CPC A61K 31/445; A61P 13/12

(57) ABSTRACT

Provided are methods of treating Fabry disease in a patient having severe renal impairment. A method for the treatment of Fabry disease in a patient having severe renal impairment, including administering to the patient about 50 mg to about 200 mg free base equivalent (FBE) of migalastat or salt thereof at a frequency of less than once every week. A method for the treatment of Fabry disease in a patient having severe renal impairment, including administering to the patient about 50 mg to about 100 mg free base equivalent of migalastat or salt thereof at a frequency of between about once every three days and about once every two weeks.

### CROSS-REFERENCE TO RELATED APPLICATIONS

This application claims the benefit of U.S. Provisional Application Ser. No. 63/696,627, filed Sep. 19, 2024, the disclosure of which is incorporated by reference in its entirety.

### REFERENCE TO THE SEQUENCE LISTING

The Sequence Listing XML file submitted herewith, identified as “AT24-004 Sequence Listing.xml” (17,760 bytes, created Sep. 19, 2025), is hereby incorporated by reference.

### TECHNICAL FIELD

Principles and embodiments of the present invention relate generally to the use of pharmacological chaperones for the treatment of Fabry disease, particularly in patients with severe renal impairment who are undergoing dialysis treatment.

### BACKGROUND

Many human diseases result from mutations that cause changes in the amino acid sequence of a protein which reduce its stability and may prevent it from folding properly. Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded proteins are often eliminated by the quality control mechanisms after initially being retained in the ER. In certain instances, misfolded proteins can accumulate in the ER before being eliminated. The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Such mutations can lead to lysosomal storage disorders (LSDs), which are characterized by deficiencies of lysosomal enzymes due to mutations in the genes encoding the lysosomal enzymes. The resultant disease causes the pathologic accumulation of substrates of those enzymes, which include lipids, carbohydrates, and polysaccharides. Although there are many different mutant genotypes associated with each LSD, many of the mutations are missense mutations which can lead to the production of a less stable enzyme. These less stable enzymes are sometimes prematurely degraded by the ER-associated degradation pathway. This results in the enzyme deficiency in the lysosome, and the pathologic accumulation of substrate. Such mutant enzymes are sometimes referred to in the pertinent art as “folding mutants” or “conformational mutants.”

Fabry Disease is a LSD caused by a mutation to the GLA gene, which encodes the enzyme α-galactosidase A (α-Gal A). α-Gal A is required for glycosphingolipid metabolism. The mutation causes the substrate globotriaosylceramide (Gb3, GL-3, or ceramide trihexoside) to accumulate in various tissues and organs. Males with Fabry disease are hemizygotes because the disease genes are encoded on the X chromosome. Fabry disease is estimated to affect 1 in 40,000 and 60,000 males, and occurs less frequently in females.

There have been several approaches to treatment of Fabry disease. One approved therapy for treating Fabry disease is enzyme replacement therapy (ERT), which typically involves intravenous, infusion of a purified form of the corresponding wild-type protein (Fabrazyme®, Genzyme Corp.). ERT has several drawbacks, however. One of the main complications with enzyme replacement therapy is rapid degradation of the infused protein, which leads to the need for numerous, costly high dose infusions. ERT has several additional caveats, such as difficulties with large-scale generation, purification, and storage of properly folded protein; obtaining glycosylated native protein; generation of an anti-protein immune response; and inability of protein to cross the blood-brain barrier to mitigate central nervous system pathologies (i.e., low bioavailability). In addition, replacement enzyme cannot penetrate the heart or kidney in sufficient amounts to reduce substrate accumulation in the renal podocytes or cardiac myocytes, which figure prominently in Fabry pathology.

Another approach to treating some enzyme deficiencies involves the use of small molecule inhibitors to reduce production of the natural substrate of deficient enzyme proteins, thereby ameliorating the pathology. This “substrate reduction” approach has been specifically described for a class of about 40 related enzyme disorders called lysosomal storage disorders that include glycosphingolipid storage disorders. The small molecule inhibitors proposed for use as therapy are specific for inhibiting the enzymes involved in synthesis of glycolipids, reducing the amount of cellular glycolipid that needs to be broken down by the deficient enzyme.

A third approach to treating Fabry disease has been treatment with what are called pharmacological chaperones (PCs). Such PCs include small molecule inhibitors of α-Gal A, which can bind to the α-Gal A to increase the stability of both mutant enzyme and the corresponding wild type.

One problem with current treatments is difficulty in treating patients exhibiting renal impairment, which is very common in Fabry patients and progresses with the disease. On average, it take between about 10-20 years for patients to decline from normal kidney function to severe renal impairment, with some countries reporting even faster declines. By some estimates, about 10% of Fabry patients receiving ERT may have moderate renal impairment. Another 25% of males and 5% of females receiving ERT have an estimated glomerular filtration rate (eGFR) of less than 30, corresponding to severe kidney impairment or even renal failure. Of these, about half have severe kidney impairment, and about half are on dialysis.

Unfortunately, renal impairment will progress despite ERT treatment. A patient having an eGFR of 30 may deteriorate to the point of needing dialysis in two to five years. About 30% of patients receiving ERT will end up on dialysis or needing a kidney transplant, depending on the start of ERT. The earlier ERT is commenced, the longer renal function may be preserved, but commencement of ERT may be delayed because Fabry disease is rare and often misdiagnosed.

Further, and as discussed above, ERT often does not sufficiently penetrate the kidneys to reduce substrate accumulation, thereby allowing further damage during disease progression. With PC treatment, the kidneys are often how the drug is cleared from the body, and renal impairment may affect drug pharmacokinetics and/or drug pharmacodynamics. Thus, there is still a need for a treatment of Fabry patients who have renal impairment.

For patients with Fabry disease receiving dialysis therapy for severe renal impairment or end stage renal disease (ESRD), consideration of dose adjustment may be important data to drive informed treatment decisions. Timely and effective interventions, including disease intervention, are needed for this subset of the FD population (Linares D, Luna B, Loayza E, Taboada G, Ramaswamu U., Presence of Fabry diseases in patients with chronic kidney disease: a systematic review and meta-analysis. Mol Genet Metab. 2023, 140 (4): 107714, which is incorporated herein by reference in its entirety). Widening treatment options in patients with FD and severe renal impairment are needed to improve the clinical care of these affected patients and provide greater treatment options.

### SUMMARY

One aspect of the invention pertains to a method for treatment of Fabry disease in a patient having renal impairment, the method comprising administering to the patient about 50 mg to about 200 mg free base equivalent (FBE) of migalastat or salt thereof at a frequency of less than once every week. In some embodiments, the frequency is once every two weeks. In one or more embodiments, the patient has severe renal impairment. In some embodiments, the patient is undergoing dialysis treatment. In some embodiments, the migalastat is in a solid dosage form. In one or more embodiments, the patient is administered about 123 mg FBE. In some embodiments, the patient is administered about 150 mg migalastat HCl. In one or more embodiments, the migalastat is administered orally. In one or more embodiments, the migalastat is administered for at least 28 days. In one or more embodiments, the migalastat is administered for at least 6 months. In one or more embodiments, the migalastat is administered for at least 12 months.

A second aspect of the invention pertains to a method for treatment of Fabry disease in a patient having renal impairment, the method comprising administering to the patient about 50 mg to about 100 mg FBE of migalastat or salt thereof at a frequency of from about once every three days to about once every two weeks. In some embodiments, the frequency is once every week. In one or more embodiments, the patient has severe renal impairment. In some embodiments, the patient is undergoing dialysis treatment. In some embodiments, the migalastat is in a solid dosage form. In one or more embodiments, the patient is administered about 82 mg FBE. In some embodiments, the patient is administered about 100 mg migalastat HCl. In one or more embodiments, the migalastat is administered orally. In one or more embodiments, the migalastat is administered for at least 28 days. In one or more embodiments, the migalastat is administered for at least 6 months. In one or more embodiments, the migalastat is administered for at least 12 months.

A third aspect of the invention pertains to the use of migalastat in the treatment of Fabry disease in a patient having renal impairment, wherein the migalastat is administered to a Fabry disease patient having renal impairment in an amount of about 50 mg to about 200 mg FBE of migalastat or salt thereof at a frequency of less than once every week. In some embodiments, the frequency is once every two weeks. In some embodiments, the patient has severe renal impairment. In some embodiments, the patient is undergoing dialysis treatment. In one or more embodiments, the migalastat is in a solid dosage form. In some embodiments, the patient is administered about 123 mg FBE. In one or more embodiments, the patient is administered about 150 mg migalastat HCl. In some embodiments, the migalastat is administered orally.

A fourth aspect of the invention pertains to the use of migalastat in the treatment of Fabry disease in a patient having renal impairment, wherein the migalastat is administered to a Fabry disease patient having renal impairment in an amount of about 50 mg to about 100 mg FBE of migalastat or salt thereof at a frequency of from about once every three days to about once every two weeks. In some embodiments, the frequency is once every week. In some embodiments, the patient has severe renal impairment. In some embodiments, the patient is undergoing dialysis treatment. In one or more embodiments, the migalastat is in a solid dosage form. In some embodiments, the patient is administered about 82 mg FBE. In one or more embodiments, the patient is administered about 100 mg migalastat HCl. In some embodiments, the migalastat is administered orally.

Various embodiments are listed below. It will be understood that the embodiments listed below may be combined not only as listed below, but in other suitable combinations in accordance with the scope of the invention.

### BRIEF DESCRIPTION OF THE DRAWINGS

FIG. 1 is a schematic flow diagram of subject disposition for a Phase 1 clinical study as described herein;

FIG. 2 is a schematic of migalastat does timing, follow-up, and sample collection for a Phase I clinical study as described herein;

FIG. 3 illustrates plasma migalastat concentration curves for subjects administered migalastat 24 hours prior to dialysis;

FIG. 4 illustrates plasma migalastat concentration curves for subjects administered migalastat immediately prior to dialysis;

FIG. 5 illustrates predicted geometric mean ratios for C_{avg} and Cₘₐₓ concentrations as calculated using a Population Pharmacokinetic Model;

FIGS. 6A and 6B illustrate predicted median steady-state plasma migalastat concentrations as calculated using a Population Pharmacokinetic Model; and

FIG. 7 illustrates predicted C_{trough} concentrations as calculated using a Population Pharmacokinetic Model.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Various aspects of the present invention pertain to particular dosing regimens of migalastat or a salt thereof for Fabry patients having renal impairment. Migalastat is a pharmacological chaperone used in the treatment of Fabry disease. This pharmacological chaperone is usually cleared from the body by the kidneys. However, patients who have renal impairment (a common problem for Fabry patients) may not be able to clear the migalastat from the body, and it was not previously known how patients with both Fabry disease and renal impairment would respond to migalastat therapy. Because pharmacological chaperones are also inhibitors, 7alanceng the enzyme-enhancing and inhibitory effects of pharmacological chaperones such as migalastat is very difficult. Moreover, due to the complex interactions between Fabry disease and renal function and the lack of knowledge on the role of a pharmacological chaperone, migalastat dosing for Fabry patients with renal impairment is difficult to ascertain without significant clinical data and/or computer modeling.

Accordingly, one aspect of the invention pertains to a method for treatment of Fabry disease in a patient having renal impairment. In some embodiments, the method comprises administering migalastat or a salt thereof every three, four, five, six or seven days. In other embodiments, the method comprises administering migalastat or a salt thereof every eight, nine, ten, eleven, twelve, thirteen, or fourteen days. In some embodiments, the method comprises administering migalastat or a salt thereof at a frequency of less than once every 14 days. Although specific reference is made to administering every one week or every two weeks, the methods and uses disclosed herein can also be used with other intermittent dosing regimens, such as every three, five or six days, or every three or four weeks, based on, for example, the state of a patient's kidneys.

In one or more embodiments, the method comprises administering to the patient about 50 mg to about 200 mg FBE of migalastat or salt thereof at a frequency of less than once every week. In some embodiments, the method comprises administering to the patient about 123 mg FBE of migalastat or salt thereof at a frequency of once every two weeks. The patient may have severe renal impairment. In some embodiments, the patient may be undergoing dialysis treatment. In some embodiments, the migalastat is administered between about 12 hours and about 36 hours prior to starting the dialysis treatment, such as about 24 hours prior to starting the dialysis treatment. In some embodiments, the dialysis comprises hemodialysis or hemodiafiltration.

In one or more embodiments, the method comprises administering to the patient about 50 mg to about 100 mg FBE of migalastat or salt thereof at a frequency of between about once every 3 days and about once every two weeks. In some embodiments, the method comprises administering to the patient about 82 mg FBE of migalastat or salt thereof at a frequency of once every week. The patient may have severe renal impairment. In some embodiments, the migalastat is administered between about 12 hours and about 36 hours prior to starting the dialysis treatment, such as about 24 hours prior to starting the dialysis treatment. In some embodiments, the dialysis comprises hemodialysis or hemodiafiltration.

Another aspect of the invention pertains to a use of migalastat in the treatment of Fabry disease in a patient having renal impairment, wherein the migalastat is administered to a Fabry disease patient having renal impairment in an amount of about 50 mg to about 200 mg FBE of migalastat or salt thereof at a frequency of less than once every week. In some embodiments, the use comprises administering to the patient about 123 mg FBE of migalastat or salt thereof at a frequency of once every two weeks. The patient may have severe renal impairment.

Another aspect of the invention pertains to a use of migalastat in the treatment of Fabry disease in a patient having renal impairment, wherein the migalastat is administered to a Fabry disease patient having renal impairment in an amount of about 50 mg to about 100 mg FBE of migalastat or salt thereof at a frequency of between about once every three days and about once every two weeks. In some embodiments, the use comprises administering to the patient about 82 mg FBE of migalastat or salt thereof at a frequency of once every week. The patient may have severe renal impairment.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase A activity. This defect causes accumulation of globotriaosylceramide (ceramide trihexoside) and related glycosphingolipids in vascular endothelial lysosomes of the heart, kidneys, skin, and other tissues.

The term “atypical Fabry disease” refers to patients with primarily cardiac manifestations of the α-Gal A deficiency, namely progressive globotriaosylceramide (GL-3) accumulation in myocardial cells that leads to significant enlargement of the heart, particularly the left ventricle.

A “carrier” is a female who has one X chromosome with a defective α-Gal A gene and one X chromosome with the normal gene and in whom X chromosome inactivation of the normal allele is present in one or more cell types. A carrier is often diagnosed with Fabry disease.

A “patient” refers to a subject who has been diagnosed with or is suspected of having a particular disease. The patient may be human or animal.

A “Fabry disease patient” refers to an individual who has been diagnosed with or suspected of having Fabry disease and has a mutated α-Gal A as defined further below. Characteristic markers of Fabry disease can occur in male hemizygotes and female carriers with the same prevalence, although females typically are less severely affected.

Human α-galactosidase A (α-Gal A) refers to an enzyme encoded by the human GLA gene. The full DNA sequence of α-Gal A, including introns and exons, is available in GenBank Accession No. X14448.1 and shown in SEQ ID NO: 1. The human α-Gal A enzyme consists of 429 amino acids and is available in GenBank Accession Nos. X14448.1 and U78027.1 and shown in SEQ ID NO: 2. The nucleic acid sequence that only includes the coding regions (i.e. exons) of SEQ ID NO: 1 is shown in SEQ ID NO: 3.

The term “mutant protein” includes a protein which has a mutation in the gene encoding the protein which results in the inability of the protein to achieve a stable conformation under the conditions normally present in the ER. The failure to achieve a stable conformation results in a substantial amount of the enzyme being degraded, rather than being transported to the lysosome. Such a mutation is sometimes called a “conformational mutant.” Such mutations include, but are not limited to, missense mutations, and in-frame small deletions and insertions.

As used herein in one embodiment, the term “mutant α-Gal A” includes an α-Gal A which has a mutation in the gene encoding α-Gal A which results in the inability of the enzyme to achieve a stable conformation under the conditions normally present in the ER. The failure to achieve a stable conformation results in a substantial amount of the enzyme being degraded, rather than being transported to the lysosome.

As used herein, the term “specific pharmacological chaperone” (“SPC”) or “pharmacological chaperone” (“PC”) refers to any molecule including a small molecule, protein, peptide, nucleic acid, carbohydrate, etc. that specifically binds to a protein and has one or more of the following effects: (i) enhances the formation of a stable molecular conformation of the protein; (ii) induces trafficking of the protein from the ER to another cellular location, preferably a native cellular location, i.e., prevents ER-associated degradation of the protein; (iii) prevents aggregation of misfolded proteins; and/or (iv) restores or enhances at least partial wild-type function and/or activity to the protein. A compound that specifically binds to e.g., α-Gal A, means that it binds to and exerts a chaperone effect on the enzyme and not a generic group of related or unrelated enzymes. More specifically, this term does not refer to endogenous chaperones, such as BiP, or to non-specific agents which have demonstrated non-specific chaperone activity against various proteins, such as glycerol, DMSO or deuterated water, i.e., chemical chaperones. In one or more embodiments of the present invention, the PC may be a reversible competitive inhibitor.

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

As used herein, the term “specifically binds” refers to the interaction of a pharmacological chaperone with a protein such as α-Gal A, specifically, an interaction with amino acid residues of the protein that directly participate in contacting the pharmacological chaperone. A pharmacological chaperone specifically binds a target protein, e.g., α-Gal A, to exert a chaperone effect on the protein and not a generic group of related or unrelated proteins. The amino acid residues of a protein that interact with any given pharmacological chaperone may or may not be within the protein's “active site.” Specific binding can be evaluated through routine binding assays or through structural studies, e.g., co-crystallization, NMR, and the like. The active site for α-Gal A is the substrate binding site.

“Deficient α-Gal A activity” refers to α-Gal A activity in cells from a patient which is below the normal range as compared (using the same methods) to the activity in normal individuals not having or suspected of having Fabry or any other disease (especially a blood disease).

As used herein, the terms “enhance α-Gal A activity” or “increase α-Gal A activity” refer to increasing the amount of α-Gal A that adopts a stable conformation in a cell contacted with a pharmacological chaperone specific for the α-Gal A, relative to the amount in a cell (preferably of the same cell-type or the same cell, e.g., at an earlier time) not contacted with the pharmacological chaperone specific for the α-Gal A. This term also refers to increasing the trafficking of α-Gal A to the lysosome in a cell contacted with a pharmacological chaperone specific for the α-Gal A, relative to the trafficking of α-Gal A not contacted with the pharmacological chaperone specific for the protein. These terms refer to both wild-type and mutant α-Gal A. In one embodiment, the increase in the amount of α-Gal A in the cell is measured by measuring the hydrolysis of an artificial substrate in lysates from cells that have been treated with the PC. An increase in hydrolysis is indicative of increased α-Gal A activity.

The term “α-Gal A activity” refers to the normal physiological function of a wild-type α-Gal A in a cell. For example, α-Gal A activity includes hydrolysis of GL-3.

A “responder” is an individual diagnosed with or suspected of having a lysosomal storage disorder, such, for example Fabry disease, whose cells exhibit sufficiently increased α-Gal A activity, respectively, and/or amelioration of symptoms or improvement in surrogate markers, in response to contact with a PC. Non-limiting examples of improvements in surrogate markers for Fabry are lyso-Gb3 and those disclosed in US Patent Application Publication No. US 2010-0113517, which is hereby incorporated by reference in its entirety.

Non-limiting examples of improvements in surrogate markers for Fabry disease disclosed in US 2010/0113517 include increases in α-Gal A levels or activity in cells (e.g., fibroblasts) and tissue; reductions in of GL-3 accumulation; decreased plasma concentrations of homocysteine and vascular cell adhesion molecule-1 (VCAM-1); decreased GL-3 accumulation within myocardial cells and valvular fibrocytes; reduction in plasma globotriaosylsphingosine (lyso-Gb3); reduction in cardiac hypertrophy (especially of the left ventricle), amelioration of valvular insufficiency, and arrhythmias; amelioration of proteinuria; decreased urinary concentrations of lipids such as CTH, lactosylceramide, ceramide, and increased urinary concentrations of glucosylceramide and sphingomyelin; the absence of laminated inclusion bodies (Zebra bodies) in glomerular epithelial cells; improvements in renal function; mitigation of hypohidrosis; the absence of angiokeratomas; and improvements hearing abnormalities such as high frequency sensorineural hearing loss progressive hearing loss, sudden deafness, or tinnitus. Improvements in neurological symptoms include prevention of transient ischemic attack (TIA) or stroke; and amelioration of neuropathic pain manifesting itself as acroparaesthesia (burning or tingling in extremities). Another type of clinical marker that can be assessed for Fabry disease is the prevalence of deleterious cardiovascular manifestations. Common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure.

The phrase “pharmaceutically acceptable” refers to molecular entities and compositions that are physiologically tolerable and do not typically produce untoward reactions when administered to a human. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” in reference to a pharmaceutical carrier refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

The term “enzyme replacement therapy” or “ERT” refers to the introduction of a non-native, purified enzyme into an individual having a deficiency in such enzyme. The administered protein can be obtained from natural sources or by recombinant expression (as described in greater detail below). The term also refers to the introduction of a purified enzyme in an individual otherwise requiring or benefiting from administration of a purified enzyme, e.g., suffering from enzyme insufficiency. The introduced enzyme may be a purified, recombinant enzyme produced in vitro, or protein purified from isolated tissue or fluid, such as, e.g., placenta or animal milk, or from plants.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

As used herein, the term “free base equivalent” or “FBE” refers to the amount of migalastat present in the migalastat or salt thereof. In other words, the term “FBE” means either an amount of migalastat free base, or the equivalent amount of migalastat free base that is provided by a salt of migalastat. For example, due to the weight of the hydrochloride salt, 150 mg of migalastat hydrochloride only provides as much migalastat as 123 mg of the free base form of migalastat. Other salts are expected to have different conversion factors, depending on the molecular weight of the salt.

The term “migalastat” encompasses migalastat free base or a pharmaceutically acceptable salt thereof (e.g., migalastat HCl), unless specifically indicated to the contrary.

As used herein, the term “severe renal impairment” includes a renal impairment wherein an eGFR level, as measured by the Cockgroft-Gault equation, is less than about 30 mL/min/1.73 m².

As used herein, the term “dialysis” has its ordinary meaning as understood by the skilled person practicing in the art of the disclosure, and includes the clinical purification of blood by external means, such as to remove excess fluids and waste products, as a substitute for the normal functioning of the kidneys. The term “dialysis” includes hemodialysis (HD) and hemodiafiltration (HDF). “Hemodialysis,” or “standard dialysis,” as used herein, has its ordinary meaning as understood by the skilled person practicing in the art of the disclosure, and includes solute removal from blood across a semi-permeable membrane by diffusion. “Hemodiafiltration,” as used herein, has its ordinary meaning as understood by the skilled person practicing in the art of the disclosure, and includes solute remove from blood across a semi-permeable membrane by convection and diffusion, such as by using positive pressure to drive water across the semi-permeable membrane such that solutes are dragged with the water movement. Generally, HDF may clear more molecules of a larger size compared with standard HD.

### Fabry Disease

Fabry disease is a rare, progressive and devastating X-linked lysosomal storage disorder. Mutations in the GLA gene result in a deficiency of the lysosomal enzyme, α-Gal A, which is required for glycosphingolipid metabolism. Beginning early in life, the reduction in α-Gal A activity results in an accumulation of glycosphingolipids, including GL-3 and plasma lyso-Gb3, and leads to the symptoms and life-limiting sequelae of Fabry disease, including pain, gastrointestinal symptoms, renal failure, cardiomyopathy, cerebrovascular events, and early mortality. Early initiation of therapy and lifelong treatment provide an opportunity to slow disease progression and prolong life expectancy.

Fabry disease encompasses a spectrum of disease severity and age of onset, although it has traditionally been divided into 2 main phenotypes, “classic” and “late-onset”. The classic phenotype has been ascribed primarily to males with undetectable to low α-Gal A activity and earlier onset of renal, cardiac and/or cerebrovascular manifestations. The late-onset phenotype has been ascribed primarily to males with higher residual α-Gal A activity and later onset of these disease manifestations. Heterozygous female carriers typically express the late-onset phenotype but depending on the pattern of X-chromosome inactivation may also display the classic phenotype.

More than 800 Fabry disease-causing GLA mutations have been identified. Approximately 60% are missense mutations, resulting in single amino acid substitutions in the α-Gal A enzyme. Missense GLA mutations often result in the production of abnormally folded and unstable forms of α-Gal A and the majority are associated with the classic phenotype. Normal cellular quality control mechanisms in the endoplasmic reticulum block the transit of these abnormal proteins to lysosomes and target them for premature degradation and elimination. Many missense mutant forms are targets for migalastat, an α-Gal A-specific pharmacological chaperone.

The clinical manifestations of Fabry disease span a broad spectrum of severity and roughly correlate with a patient's residual α-GAL levels. The majority of currently treated patients are referred to as classic Fabry disease patients, most of whom are males. These patients experience disease of various organs, including the kidneys, heart and brain, with disease symptoms first appearing in adolescence and typically progressing in severity until death in the fourth or fifth decade of life. A number of recent studies suggest that there are a large number of undiagnosed males and females that have a range of Fabry disease symptoms, such as impaired cardiac or renal function and strokes, that usually first appear in adulthood. Individuals with this type of Fabry disease, referred to as later-onset Fabry disease, tend to have higher residual α-GAL levels than classic Fabry disease patients. Individuals with later-onset Fabry disease typically first experience disease symptoms in adulthood, and often have disease symptoms focused on a single organ, such as enlargement of the left ventricle or progressive kidney failure. In addition, later-onset Fabry disease may also present in the form of strokes of unknown cause.

Fabry patients have progressive kidney impairment, and untreated patients exhibit end-stage renal impairment by the fifth decade of life. Deficiency in α-Gal A activity leads to accumulation of globotriaosylceramide (Gb3) and related glycosphingolipids in many cell types including cells in the kidney. Gb3 accumulates in podocytes, epithelial cells and the tubular cells of the distal tubule and loop of Henle. Impairment in kidney function can manifest as proteinuria and reduced glomerular filtration rate.

Because Fabry disease can cause progressive worsening in renal function, it is important to understand the pharmacokinetics (PK) of potential therapeutic agents in individuals with renal impairment and particularly so for therapeutic agents that are predominantly cleared by renal excretion. Impairment of renal function may lead to accumulation of the therapeutic agent to levels that become toxic.

Because Fabry disease is rare, involves multiple organs, has a wide age range of onset, and is heterogeneous, proper diagnosis is a challenge. Awareness is low among health care professionals and misdiagnoses are frequent. Diagnosis of Fabry disease is most often confirmed on the basis of decreased α-Gal A activity in plasma or peripheral leukocytes (WBCs) once a patient is symptomatic, coupled with mutational analysis. In females, diagnosis is even more challenging since the enzymatic identification of carrier females is less reliable due to random X-chromosomal inactivation in some cells of carriers. For example, some obligate carriers (daughters of classically affected males) have α-Gal A enzyme activities ranging from normal to very low activities. Since carriers can have normal α-Gal A enzyme activity in leukocytes, only the identification of an α-Gal A mutation by genetic testing provides precise carrier identification and/or diagnosis.

Mutant forms of α-galactosidase A are considered to be amenable to migalastat are defined as showing a relative increase (+10 μM migalastat) of ≥1.20-fold and an absolute increase (+10 μM migalastat) of ≥3.0% wild-type (WT) when the mutant form of α-galactosidase A is expressed in HEK-293 cells (referred to as the “HEK assay”) according to Good Laboratory Practice (GLP)-validated in vitro assay (GLP HEK or Migalastat Amenability Assay). Such mutations are also referred to herein as “HEK assay amenable” mutations.

Previous screening methods have been provided that assess enzyme enhancement prior to the initiation of treatment. For example, an assay using HEK-293 cells has been utilized in clinical trials to predict whether a given mutation will be responsive to pharmacological chaperone (e.g., migalastat) treatment. In this assay, cDNA constructs are created. The corresponding α-Gal A mutant forms are transiently expressed in HEK-293 cells. Cells are then incubated±migalastat (17 nM to 1 mM) for 4 to 5 days. After, α-Gal A levels are measured in cell lysates using a synthetic fluorogenic substrate (4-MU-α-Gal) or by western blot. This has been done for known disease-causing missense or small in-frame insertion/deletion mutations. Mutations that have previously been identified as responsive to a PC (e.g. migalastat) using these methods are listed in U.S. Pat. No. 8,592,362, which is hereby incorporated by reference in its entirety.

### Pharmacological Chaperones

The binding of small molecule inhibitors of enzymes associated with LSDs can increase the stability of both mutant enzyme and the corresponding wild-type enzyme (see U.S. Pat. Nos. 6,274,597; 6,583,158; 6,589,964; 6,599,919; 6,916,829, and 7,141,582 all incorporated herein by reference). In particular, administration of small molecule derivatives of glucose and galactose, which are specific, selective competitive inhibitors for several target lysosomal enzymes, effectively increased the stability of the enzymes in cells in vitro and, thus, increased trafficking of the enzymes to the lysosome. Thus, by increasing the amount of enzyme in the lysosome, hydrolysis of the enzyme substrates is expected to increase. The original theory behind this strategy was as follows: since the mutant enzyme protein is unstable in the ER (Ishii et al., Biochem. Biophys. Res. Comm. 1996; 220:812-815), the enzyme protein is retarded in the normal transport pathway (ER→Golgi apparatus→endosomes→lysosome) and prematurely degraded. Therefore, a compound which binds to and increases the stability of a mutant enzyme, may serve as a “chaperone” for the enzyme and increase the amount that can exit the ER and move to the lysosomes. In addition, because the folding and trafficking of some wild-type proteins is incomplete, with up to 70% of some wild-type proteins being degraded in some instances prior to reaching their final cellular location, the chaperones can be used to stabilize wild-type enzymes and increase the amount of enzyme which can exit the ER and be trafficked to lysosomes.

In one or more embodiments, the pharmacological chaperone comprises migalastat or a salt thereof. The compound migalastat, also known as 1-deoxygalactonojirimycin (1-DGJ) or (2R,3S,4R,5S)-2-(hydroxymethyl) piperdine-3,4,5-triol is a compound having the following chemical formula:

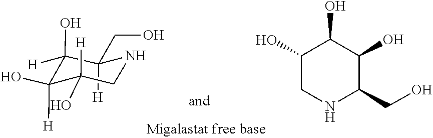 [SMILES: OCC1NCC(O)C(O)C1O ; OC[C@H]1NC[C@H](O)[C@@H](O)[C@H]1O]

As discussed herein, pharmaceutically acceptable salts of migalastat may also be used in the present invention. When a salt of migalastat is used, the dosage of the salt will be adjusted so that the dose of migalastat received by the patient is equivalent to the amount which would have been received had the migalastat free base been used. One example of a pharmaceutically acceptable salt of migalastat is migalastat HCl:

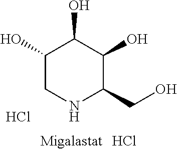 [SMILES: OC[C@H]1NC[C@H](O)[C@@H](O)[C@H]1O ; Cl ; Cl]

Migalastat is a low molecular weight iminosugar and is an analogue of the terminal galactose of GL-3. In vitro and in vivo pharmacologic studies have demonstrated that migalastat acts as a pharmacological chaperone, selectively and reversibly binding, with high affinity, to the active site of wild-type (WT) α-Gal A and specific mutant forms of a Gal A, the genotypes of which are referred to as HEK assay amenable mutations. Migalastat binding stabilizes these mutant forms of α-Gal A in the endoplasmic reticulum facilitating their proper trafficking to lysosomes where dissociation of migalastat allows α-Gal A to reduce the level of GL-3 and other substrates. Approximately 30-50% of patients with Fabry disease have HEK assay amenable mutations; the majority of which are associated with the classic phenotype of the disease. A list of HEK assay amenable mutations includes at least those mutations listed in Table 1 below. In one or more embodiments, if a double mutation is present on the same chromosome (males and females), that patient is considered HEK assay amenable if the double mutation is present in one entry in Table 1 (e.g., D55V/Q57L). In some embodiments, if a double mutation is present on different chromosomes (only in females) that patient is considered HEK assay amenable if either one of the individual mutations is present in Table 1. In addition to Table 1 below, HEK assay amenable mutations can also be found in the summary of product characteristics and/or prescribing information for GALAFOLD™ in various countries in which GALAFOLD™ is approved for use, or at the website www.galafoldamenabilitytable.com, each of which is hereby incorporated by reference in its entirety.

TABLE 1
Amenable mutations
Table 1

| | | Protein |
|---|---|---|
| Nucleotide | Nucleotide | sequence |
| change | change | change |
| c.7C > G | c.C7G | L3V |
| c.8T > C | c.T8C | L3P |
| c.[11G > T; | c.G11T/A620C | R4M/Y207S |
| 620A > C] | | |
| c.13A > G | c.A13G | N5D |
| c.15C > G | c.C15G | N5K |
| c.16C > A | c.C16A | P6T |
| c.16C > T | c.C16T | P6S |
| c.17C > A | c.C17A | P6Q |
| c.17C > G | c.C17G | P6R |
| c.17C > T | c.C17T | P6L |
| c.19G > A | c.G19A | E7K |
| c.20A > T | c.A20T | E7V |
| c.21A > T | c.A21T | E7D |
| c.22C > A | c.C22A | L8I |
| c.23T > A | c.T23A | L8Q |
| c.23T > C | c.T23C | L8P |
| c.25C > T | c.C25T | H9Y |
| c.26A > G | c.A26G | H9R |
| c.26A > T | c.A26T | H9L |
| c.27T > A | c.T27A | H9Q |
| c.28C > A | c.C28A | L10M |
| c.28C > G | c.C28G | L10V |
| c.29T > A | c.T29A | L10Q |
| c.29T > C | c.T29C | L10P |
| c.29T > G | c.T29G | L10R |
| c.31G > A | c.G31A | G11S |
| c.31G > C | c.G31C | G11R |
| c.31G > T | c.G31T | G11C |
| c.32G > A | c.G32A | G11D |
| c.32G > T | c.G32T | G11V |
| c.34T > A | c.T34A | C12S |
| c.34T > C | c.T34C | C12R |
| c.34T > G | c.T34G | C12G |
| c.35G > A | c.G35A | C12Y |
| c.37G > A | c.G37A | A13T |
| c.37G > C | c.G37C | A13P |
| c.38C > A | c.C38A | A13E |
| c.38C > G | c.C38G | A13G |
| c.40C > G | c.C40G | L14V |
| c.40C > T | c.C40T | L14F |
| c.41T > A | c.T41A | L14H |
| c.43G > A | c.G43A | A15T |
| c.44C > G | c.C44G | A15G |
| c.49C > A | c.C49A | R17S |
| c.49C > G | c.C49G | R17G |
| c.49C > T | c.C49T | R17C |
| c.50G > A | c.G50A | R17H |
| c.50G > C | c.G50C | R17P |
| c.52T > A | c.T52A | F18I |
| c.53T > G | c.T53G | F18C |
| c.54C > G | c.C54G | F18L |
| c.58G > C | c.G58C | A20P |
| c.59C > A | c.C59A | A20D |
| c.59C > G | c.C59G | A20G |
| c.62T > A | c.T62A | L21H |
| c.64G > A | c.G64A | V22I |
| c.64G > C | c.G64C | V22L |
| c.64G > T | c.G64T | V22F |
| c.65T > C | c.T65C | V22A |
| c.65T > G | c.T65G | V22G |
| c.67T > A | c.T67A | S23T |
| c.67T > C | c.T67C | S23P |
| c.70T > C or | c.T70C or | W24R |
| c.70T > A | c.T70A | |
| c.70T > G | c.T70G | W24G |
| c.71G > C | c.G71C | W24S |
| c.72G > C or | c.G72C or | W24C |
| c.72G > T | c.G72T | |
| c.73G > C | c.G73C | D25H |
| c.77T > A | c.T77A | I26N |
| c.79C > A | c.C79A | P27T |
| c.79C > G | c.C79G | P27A |
| c.79C > T | c.C79T | P27S |
| c.80C > T | c.C80T | P27L |
| c.82G > C | c.G82C | G28R |
| c.82G > T | c.G82T | G28W |
| c.83G > A | c.G83A | G28E |
| c.85G > C | c.G85C | A29P |
| c.86C > A | c.C86A | A29D |
| c.86C > G | c.C86G | A29G |
| c.86C > T | c.C86T | A29V |
| c.88A > G | c.A88G | R30G |
| c.94C > A | c.C94A | L32M |
| c.94C > G | c.C94G | L32V |
| c.95T > A | c.T95A | L32Q |
| c.95T > C | c.T95C | L32P |
| c.95T > G | c.T95G | L32R |
| c.97G > C | c.G97C | D33H |
| c.97G > T | c.G97T | D33Y |
| c.98A > C | c.A98C | D33A |
| c.98A > G | c.A98G | D33G |
| c.98A > T | c.A98T | D33V |
| c.99C > G | c.C99G | D33E |
| c.100A > C | c.A100C | N34H |
| c.100A > G | c.A100G | N34D |
| c.101A > C | c.A101C | N34T |
| c.101A > G | c.A101G | N34S |
| c.102T > G or | c.T102G or | N34K |
| c.102T > A | c.T102A | |
| c.103G > C or | c.G103C or | G35R |
| c.103G > A | c.G103A | |
| c.104G > A | c.G104A | G35E |
| c.104G > C | c.G104C | G35A |
| c.104G > T | c.G104T | G35V |
| c.106T > A | c.T106A | L36M |
| c.106T > G | c.T106G | L36V |
| c.107T > C | c.T107C | L36S |
| c.107T > G | c.T107G | L36W |
| c.108G > C or | c.G108C or | L36F |
| c.108G > T | c.G108T | |
| c.109G > A | c.G109A | A37T |
| c.109G > T | c.G109T | A37S |
| c.110C > A | c.C110A | A37E |
| c.110C > G | c.C110G | A37G |
| c.110C > T | c.C110T | A37V |
| c.112A > G | c.A112G | R38G |
| c.112A > T | c.A112T | R38W |
| c.113G > T | c.G113T | R38M |
| c.114G > C | c.G114C | R38S |
| c.115A > G | c.A115G | T39A |
| c.115A > T | c.A115T | T39S |
| c.116C > A | c.C116A | T39K |
| c.116C > G | c.C116G | T39R |
| c.116C > T | c.C116T | T39M |
| c.121A > G | c.A121G | T41A |
| c.122C > A | c.C122A | T41N |
| c.122C > G | c.C122G | T41S |
| c.122C > T | c.C122T | T41I |
| c.124A > C or | c.A124C or | M42L |
| c.124A > T | c.A124T | |
| c.124A > G | c.A124G | M42V |
| c.125T > A | c.T125A | M42K |
| c.125T > C | c.T125C | M42T |
| c.125T > G | c.T125G | M42R |
| c.126G > A or | c.G126A or | M42I |
| c.126G > C or | c.G126C or | |
| c.126G > T | c.G126T | |
| c.128G > C | c.G128C | G43A |
| c.133C > A | c.C133A | L45M |
| c.133C > G | c.C133G | L45V |
| c.136C > A | c.C136A | H46N |
| c.136C > G | c.C136G | H46D |
| c.137A > C | c.A137C | H46P |
| c.138C > G | c.C138G | H46Q |
| c.142G > C | c.G142C | E48Q |
| c.143A > C | c.A143C | E48A |
| c.149T > A | c.T149A | F50Y |
| c.151A > G | c.A151G | M51V |
| c.152T > A | c.T152A | M51K |
| c.152T > C | c.T152C | M51T |
| c.152T > G | c.T152G | M51R |
| c.153G > A or | c.G153A or | M51I |
| c.153G > T or | c.G153T or | |
| c.153G > C | c.G153C | |
| c.157A > C | c.A157C | N53H |
| c.[157A > C; | c.A157C/A158T | N53L |
| 158A > T] | | |
| c.157A > G | c.A157G | N53D |
| c.157A > T | c.A157T | N53Y |
| c.158A > C | c.A158C | N53T |
| c.158A > G | c.A158G | N53S |
| c.158A > T | c.A158T | N53I |
| c.159C > G or | c.C159G or | N53K |
| c.159C > A | c.C159A | |
| c.160C > G | c.C160G | L54V |
| c.160C > T | c.C160T | L54F |
| c.161T > A | c.T161A | L54H |
| c.161T > C | c.T161C | L54P |
| c.161T > G | c.T161G | L54R |
| c.163G > C | c.G163C | D55H |
| c.163G > T | c.G163T | D55Y |
| c.164A > C | c.A164C | D55A |
| c.164A > G | c.A164G | D55G |
| c.164A > T | c.A164T | D55V |
| c.[164A > T; | c.A164T/A170T | D55V/Q57L |
| 170A > T] | | |
| c.165C > G | c.C165G | D55E |
| c.167G > A | c.G167A | C56Y |
| c.167G > T | c.G167T | C56F |
| c.168C > G | c.C168G | C56W |
| c.170A > G | c.A170G | Q57R |
| c.170A > T | c.A170T | Q57L |
| c.172G > A | c.G172A | E58K |
| c.175G > A | c.G175A | E59K |
| c.175G > C | c.G175C | E59Q |
| c.176A > C | c.A176C | E59A |
| c.176A > G | c.A176G | E59G |
| c.176A > T | c.A176T | E59V |
| c.177G > C | c.G177C | E59D |
| c.178C > A | c.C178A | P60T |
| c.178C > G | c.C178G | P60A |
| c.178C > T | c.C178T | P60S |
| c.179C > A | c.C179A | P60Q |
| c.179C > G | c.C179G | P60R |
| c.179C > T | c.C179T | P60L |
| c.182A > T | c.A182T | D61V |
| c.183T > A | c.T183A | D61E |
| c.184_185insTAG | c.184_185insTAG | S62delinsLA |
| c.184T > C | c.T184C | S62P |
| c.184T > G | c.T184G | S62A |
| c.185C > A | c.C185A | S62Y |
| c.185C > G | c.C185G | S62C |
| c.185C > T | c.C185T | S62F |
| c.190A > C | c.A190C | I64L |
| c.190A > G | c.A190G | I64V |
| c.193A > G | c.A193G | S65G |
| c.193A > T | c.A193T | S65C |
| c.195T > A | c.T195A | S65R |
| c.196G > A | c.G196A | E66K |
| c.197A > G | c.A197G | E66G |
| c.197A > T | c.A197T | E66V |
| c.198G > C | c.G198C | E66D |
| c.199A > C | c.A199C | K67Q |
| c.199A > G | c.A199G | K67E |
| c.200A > C | c.A200C | K67T |
| c.200A > T | c.A200T | K67M |
| c.201G > C | c.G201C | K67N |
| c.202C > A | c.C202A | L68I |
| c.205T > A | c.T205A | F69I |
| c.206T > A | c.T206A | F69Y |
| c.207C > A or | c.C207A or | F69L |
| c.207C > G | c.C207G | |
| c.208A > T | c.A208T | M70L |
| c.209T > A | c.T209A | M70K |
| c.209T > G | c.T209G | M70R |
| c.210G > C | c.G210C | M70I |
| c.211G > C | c.G211C | E71Q |
| c.212A > C | c.A212C | E71A |
| c.212A > G | c.A212G | E71G |
| c.212A > T | c.A212T | E71V |
| c.213G > C | c.G213C | E71D |
| c.214A > G | c.A214G | M72V |
| c.214A > T | c.A214T | M72L |
| c.215T > C | c.T215C | M72T |
| c.216G > A or | c.G216A or | M72I |
| c.216G > T or | c.G216T or | |
| c.216G > C | c.G216C | |
| c.217G > A | c.G217A | A73T |
| c.217G > T | c.G217T | A73S |
| c.218C > T | c.C218T | A73V |
| c.220G > A | c.G220A | E74K |
| c.221A > G | c.A221G | E74G |
| c.221A > T | c.A221T | E74V |
| c.222G > C | c.G222C | E74D |
| c.223C > T | c.C223T | L75F |
| c.224T > C | c.T224C | L75P |
| c.226A > G | c.A226G | M76V |
| c.227T > C | c.T227C | M76T |
| c.229G > A | c.G229A | V77I |
| c.229G > C | c.G229C | V77L |
| c.232T > C | c.T232C | S78P |
| c.233C > T | c.C233T | S78L |
| c.235G > A | c.G235A | E79K |
| c.235G > C | c.G235C | E79Q |
| c.236A > C | c.A236C | E79A |
| c.236A > G | c.A236G | E79G |
| c.236A > T | c.A236T | E79V |
| c.237A > T | c.A237T | E79D |
| c.238G > A | c.G238A | G80S |
| c.238G > T | c.G238T | G80C |
| c.239G > A | c.G239A | G80D |
| c.239G > C | c.G239C | G80A |
| c.239G > T | c.G239T | G80V |
| c.242G > T | c.G242T | W81L |
| c.244A > G | c.A244G | K82E |
| c.245A > C | c.A245C | K82T |
| c.245A > G | c.A245G | K82R |
| c.245A > T | c.A245T | K82M |
| c.246G > C | c.G246C | K82N |
| c.247G > A | c.G247A | D83N |
| c.248A > C | c.A248C | D83A |
| c.248A > G | c.A248G | D83G |
| c.248A > T | c.A248T | D83V |
| c.249T > A | c.T249A | D83E |
| c.250G > A | c.G250A | A84T |
| c.250G > C | c.G250C | A84P |
| c.250G > T | c.G250T | A84S |
| c.251C > A | c.C251A | A84E |
| c.251C > G | c.C251G | A84G |
| c.251C > T | c.C251T | A84V |
| c.253G > A | c.G253A | G85S |
| c.[253G > A; | c.G253A/G254A | G85N |
| 254G > A] | | |
| c.[253G > A; | c.G253A/G254T/ | G85M |
| 254G > T; 255T > G] | T255G | |
| c.253G > C | c.G253C | G85R |
| c.253G > T | c.G253T | G85C |
| c.254G > A | c.G254A | G85D |
| c.254G > C | c.G254C | G85A |
| c.257A > T | c.A257T | Y86F |
| c.260A > G | c.A260G | E87G |
| c.261G > C or | c.G261C or | E87D |
| c.261G > T | c.G261T | |
| c.262T > A | c.T262A | Y88N |
| c.262T > C | c.T262C | Y88H |
| c.263A > C | c.A263C | Y88S |
| c.263A > G | c.A263G | Y88C |
| c.265C > G | c.C265G | L89V |
| c.265C > T | c.C265T | L89F |
| c.271A > C | c.A271C | I91L |
| c.271A > T | c.A271T | I91F |
| c.272T > C | c.T272C | I91T |
| c.272T > G | c.T272G | I91S |
| c.273T > G | c.T273G | I91M |
| c.286A > G | c.A286G | M96V |
| c.286A > T | c.A286T | M96L |
| c.287T > C | c.T287C | M96T |
| c.288G > A or | c.G288A or | M96I |
| c.288G > T or | c.G288T or | |
| c.288G > C | c.G288C | |
| c.289G > A | c.G289A | A97T |
| c.289G > C | c.G289C | A97P |
| c.289G > T | c.G289T | A97S |
| c.290C > A | c.C290A | A97D |
| c.290C > T | c.C290T | A97V |
| c.293C > A | c.C293A | P98H |
| c.293C > G | c.C293G | P98R |
| c.293C > T | c.C293T | P98L |
| c.295C > G | c.C295G | Q99E |
| c.296A > C | c.A296C | Q99P |
| c.296A > G | c.A296G | Q99R |
| c.296A > T | c.A296T | Q99L |
| c.301G > C | c.G301C | D101H |
| c.302A > C | c.A302C | D101A |
| c.302A > G | c.A302G | D101G |
| c.302A > T | c.A302T | D101V |
| c.303T > A | c.T303A | D101E |
| c.304T > A | c.T304A | S102T |
| c.304T > C | c.T304C | S102P |
| c.304T > G | c.T304G | S102A |
| c.305C > T | c.C305T | S102L |
| c.310G > A | c.G310A | G104S |
| c.311G > A | c.G311A | G104D |
| c.311G > C | c.G311C | G104A |
| c.311G > T | c.G311T | G104V |
| c.313A > G | c.A313G | R105G |
| c.314G > A | c.G314A | R105K |
| c.314G > C | c.G314C | R105T |
| c.314G > T | c.G314T | R105I |
| c.316C > A | c.C316A | L106I |
| c.316C > G | c.C316G | L106V |
| c.316C > T | c.C316T | L106F |
| c.317T > A | c.T317A | L106H |
| c.317T > C | c.T317C | L106P |
| c.319C > A | c.C319A | Q107K |
| c.319C > G | c.C319G | Q107E |
| c.320A > G | c.A320G | Q107R |
| c.321G > C | c.G321C | Q107H |
| c.322G > A | c.G322A | A108T |
| c.323C > A | c.C323A | A108E |
| c.323C > T | c.C323T | A108V |
| c.325G > A | c.G325A | D109N |
| c.325G > C | c.G325C | D109H |
| c.325G > T | c.G325T | D109Y |
| c.326A > C | c.A326C | D109A |
| c.326A > G | c.A326G | D109G |
| c.327C > G | c.C327G | D109E |
| c.328C > A | c.C328A | P110T |
| c.334C > G | c.C334G | R112G |
| c.335G > A | c.G335A | R112H |
| c.335G > T | c.G335T | R112L |
| c.337T > A | c.T337A | F113I |
| c.337T > C or | c.T337C or | F113L |
| c.339T > A or | c.T339A or | |
| c.339T > G | c.T339G | |
| c.337T > G | c.T337G | F113V |
| c.338T > A | c.T338A | F113Y |
| c.341C > T | c.C341T | P114L |
| c.343C > A | c.C343A | H115N |
| c.343C > G | c.C343G | H115D |
| c.346G > C | c.G346C | G116R |
| c.350T > C | c.T350C | I117T |
| c.351T > G | c.T351G | I117M |
| c.352C > T | c.C352T | R118C |
| c.361G > A | c.G361A | A121T |
| c.362C > T | c.C362T | A121V |
| c.367T > A | c.T367A | Y123N |
| c.367T > G | c.T367G | Y123D |
| c.368A > C | c.A368C | Y123S |
| c.368A > G | c.A368G | Y123C |
| c.368A > T | c.A368T | Y123F |
| c.370G > A | c.G370A | V124I |
| c.371T > G | c.T371G | V124G |
| c.373C > A | c.C373A | H125N |
| c.373C > G | c.C373G | H125D |
| c.373C > T | c.C373T | H125Y |
| c.374A > G | c.A374G | H125R |
| c.374A > T | c.A374T | H125L |
| c.376A > G | c.A376G | S126G |
| c.376A > T | c.A376T | S126C |
| c.377G > T | c.G377T | S126I |
| c.379A > G | c.A379G | K127E |
| c.383G > A | c.G383A | G128E |
| c.383G > C | c.G383C | G128A |
| c.385C > G | c.C385G | L129V |
| c.388A > C | c.A388C | K130Q |
| c.389A > T | c.A389T | K130M |
| c.390G > C | c.G390C | K130N |
| c.391C > G | c.C391G | L131V |
| c.397A > C | c.A397C | I133L |
| c.397A > G | c.A397G | I133V |
| c.397A > T | c.A397T | I133F |
| c.398T > C | c.T398C | I133T |
| c.399T > G | c.T399G | I133M |
| c.[399T > G; | c.T399G/T434C | I133M/F145S |
| 434T > C] | | |
| c.403G > A | c.G403A | A135T |
| c.403G > T | c.G403T | A135S |
| c.404C > A | c.C404A | A135E |
| c.404C > G | c.C404G | A135G |
| c.404C > T | c.C404T | A135V |
| c.406G > A | c.G406A | D136N |
| c.407A > C | c.A407C | D136A |
| c.407A > T | c.A407T | D136V |
| c.408T > A or | c.T408A or | D136E |
| c.408T > G | c.T408G | |
| c.409G > A | c.G409A | V137I |
| c.409G > C | c.G409C | V137L |
| c.410T > A | c.T410A | V137D |
| c.410T > C | c.T410C | V137A |
| c.410T > G | c.T410G | V137G |
| c.413G > C | c.G413C | G138A |
| c.415A > C | c.A415C | N139H |
| c.415A > T | c.A415T | N139Y |
| c.416A > G | c.A416G | N139S |
| c.416A > T | c.A416T | N139I |
| c.417T > A | c.T417A | N139K |
| c.418A > C | c.A418C | K140Q |
| c.418A > G | c.A418G | K140E |
| c.419A > C | c.A419C | K140T |
| c.419A > G | c.A419G | K140R |
| c.419A > T | c.A419T | K140I |
| c.420A > T | c.A420T | K140N |
| c.421A > T | c.A421T | T141S |
| c.427G > A | c.G427A | A143T |
| c.428C > A | c.C428A | A143E |
| c.428C > G | c.C428G | A143G |
| c.428C > T | c.C428T | A143V |
| c.430G > A | c.G430A | G144S |
| c.430G > C | c.G430C | G144R |
| c.430G > T | c.G430T | G144C |
| c.431G > A | c.G431A | G144D |
| c.431G > C | c.G431C | G144A |
| c.431G > T | c.G431T | G144V |
| c.433T > G | c.T433G | F145V |
| c.434T > A | c.T434A | F145Y |
| c.434T > C | c.T434C | F145S |
| c.434T > G | c.T434G | F145C |
| c.435C > G | c.C435G | F145L |
| c.436C > A | c.C436A | P146T |
| c.436C > G | c.C436G | P146A |
| c.436C > T | c.C436T | P146S |
| c.437C > A | c.C437A | P146H |
| c.437C > G | c.C437G | P146R |
| c.437C > T | c.C437T | P146L |
| c.440G > C | c.G440C | G147A |
| c.442A > G | c.A442G | S148G |
| c.442A > T | c.A442T | S148C |
| c.443G > C | c.G443C | S148T |
| c.446T > G | c.T446G | F149C |
| c.449G > A | c.G449A | G150E |
| c.449G > T | c.G449T | G150V |
| c.451T > G | c.T451G | Y151D |
| c.452A > C | c.A452C | Y151S |
| c.452A > G | c.A452G | Y151C |
| c.454T > A | c.T454A | Y152N |
| c.454T > C | c.T454C | Y152H |
| c.454T > G | c.T454G | Y152D |
| c.455A > C | c.A455C | Y152S |
| c.455A > G | c.A455G | Y152C |
| c.455A > T | c.A455T | Y152F |
| c.457G > A | c.G457A | D153N |
| c.457G > C | c.G457C | D153H |
| c.457G > T | c.G457T | D153Y |
| c.458A > C | c.A458C | D153A |
| c.458A > T | c.A458T | D153V |
| c.465T > A or | c.T465A or | D155E |
| c.465T > G | c.T465G | |
| c.466G > A | c.G466A | A156T |
| c.466G > T | c.G466T | A156S |
| c.467C > G | c.C467G | A156G |
| c.467C > T | c.C467T | A156V |
| c.469C > A | c.C469A | Q157K |
| c.469C > G | c.C469G | Q157E |
| c.470A > C | c.A470C | Q157P |
| c.470A > T | c.A470T | Q157L |
| c.471G > C or | c.G471C or | Q157H |
| c.471G > T | c.G471T | |
| c.472A > G | c.A472G | T158A |
| c.472A > T | c.A472T | T158S |
| c.473C > A | c.C473A | T158N |
| c.473C > T | c.C473T | T158I |
| c.475T > A | c.T475A | F159I |
| c.475T > G | c.T475G | F159V |
| c.476T > A | c.T476A | F159Y |
| c.476T > G | c.T476G | F159C |
| c.477T > A | c.T477A | F159L |
| c.478G > A | c.G478A | A160T |
| c.478G > T | c.G478T | A160S |
| c.479C > A | c.C479A | A160D |
| c.479C > G | c.C479G | A160G |
| c.479C > T | c.C479T | A160V |
| c.481G > A | c.G481A | D161N |
| c.481G > C | c.G481C | D161H |
| c.481G > T | c.G481T | D161Y |
| c.482A > T | c.A482T | D161V |
| c.484T > G | c.T484G | W162G |
| c.485G > C | c.G485C | W162S |
| c.490G > A | c.G490A | V164I |
| c.490G > T | c.G490T | V164L |
| c.491T > C | c.T491C | V164A |
| c.493G > A | c.G493A | D165N |
| c.493G > C | c.G493C | D165H |
| c.494A > C | c.A494C | D165A |
| c.494A > G | c.A494G | D165G |
| c.495T > A | c.T495A | D165E |
| c.496_497delinsTC | c.496_497delinsTC | L166S |
| c.496C > A | c.C496A | L166M |
| c.496C > G | c.C496G | L166V |
| c.[496C > G; | c.C496G/T497G | L166G |
| 497T > G] | | |
| c.497T > A | c.T497A | L166Q |
| c.499C > A | c.C499A | L167I |
| c.499C > G | c.C499G | L167V |
| c.505T > A | c.T505A | F169I |
| c.505T > G | c.T505G | F169V |
| c.506T > A | c.T506A | F169Y |
| c.506T > C | c.T506C | F169S |
| c.506T > G | c.T506G | F169C |
| c.507T > A | c.T507A | F169L |
| c.511G > A | c.G511A | G171S |
| c.512G > C | c.G512C | G171A |
| c.512G > T | c.G512T | G171V |
| c.517T > C | c.T517C | Y173H |
| c.518A > C | c.A518C | Y173S |
| c.518A > G | c.A518G | Y173C |
| c.518A > T | c.A518T | Y173F |
| c.520T > C | c.T520C | C174R |
| c.520T > G | c.T520G | C174G |
| c.523G > C | c.G523C | D175H |
| c.523G > T | c.G523T | D175Y |
| c.524A > G | c.A524G | D175G |
| c.524A > T | c.A524T | D175V |
| c.525C > G or | c.C525G or | D175E |
| c.525C > A | c.C525A | |
| c.526A > T | c.A526T | S176C |
| c.528T > A | c.T528A | S176R |
| c.529T > A | c.T529A | L177M |
| c.529T > G | c.T529G | L177V |
| c.530T > C | c.T530C | L177S |
| c.530T > G | c.T530G | L177W |
| c.531G > C | c.G531C | L177F |
| c.532G > A | c.G532A | E178K |
| c.532G > C | c.G532C | E178Q |
| c.533A > C | c.A533C | E178A |
| c.533A > G | c.A533G | E178G |
| c.538T > A | c.T538A | L180M |
| c.538T > G | c.T538G | L180V |
| c.539T > C | c.T539C | L180S |
| c.539T > G | c.T539G | L180W |
| c.540G > C or | c.G540C or | L180F |
| c.540G > T | c.G540T | |
| c.541G > A | c.G541A | A181T |
| c.541G > C | c.G541C | A181P |
| c.542C > T | c.C542T | A181V |
| c.544G > T | c.G544T | D182Y |
| c.545A > C | c.A545C | D182A |
| c.545A > G | c.A545G | D182G |
| c.545A > T | c.A545T | D182V |
| c.546T > A | c.T546A | D182E |
| c.548G > A | c.G548A | G183D |
| c.548G > C | c.G548C | G183A |
| c.550T > A | c.T550A | Y184N |
| c.550T > C | c.T550C | Y184H |
| c.551A > C | c.A551C | Y184S |
| c.551A > G | c.A551G | Y184C |
| c.551A > T | c.A551T | Y184F |
| c.553A > C | c.A553C | K185Q |
| c.553A > G | c.A553G | K185E |
| c.554A > C | c.A554C | K185T |
| c.554A > T | c.A554T | K185M |
| c.555G > C | c.G555C | K185N |
| c.556C > A | c.C556A | H186N |
| c.556C > G | c.C556G | H186D |
| c.556C > T | c.C556T | H186Y |
| c.557A > T | c.A557T | H186L |
| c.558C > G | c.C558G | H186Q |
| c.559_564dup | c.559_564dup | p.M187_S188dup |
| c.559A > T | c.A559T | M187L |
| c.559A > G | c.A559G | M187V |
| c.560T > C | c.T560C | M187T |
| c.561G > T or | c.G561T or | M187I |
| c.561G > A or | c.G561A or | |
| c.561G > C | c.G561C | |
| c.562T > A | c.T562A | S188T |
| c.562T > C | c.T562C | S188P |
| c.562T > G | c.T562G | S188A |
| c.563C > A | c.C563A | S188Y |
| c.563C > G | c.C563G | S188C |
| c.563C > T | c.C563T | S188F |
| c.565T > G | c.T565G | L189V |
| c.566T > C | c.T566C | L189S |
| c.567G > C or | c.G567C or | L189F |
| c.567G > T | c.G567T | |
| c.568G > A | c.G568A | A190T |
| c.568G > T | c.G568T | A190S |
| c.569C > A | c.C569A | A190D |
| c.569C > G | c.C569G | A190G |
| c.569C > T | c.C569T | A190V |
| c.571C > A | c.C571A | L191M |
| c.571C > G | c.C571G | L191V |
| c.572T > A | c.T572A | L191Q |
| c.574A > C | c.A574C | N192H |
| c.574A > G | c.A574G | N192D |
| c.575A > C | c.A575C | N192T |
| c.575A > G | c.A575G | N192S |
| c.576T > A | c.T576A | N192K |
| c.577A > G | c.A577G | R193G |
| c.577A > T | c.A577T | R193W |
| c.578G > C | c.G578C | R193T |
| c.578G > T | c.G578T | R193M |
| c.580A > C | c.A580C | T194P |
| c.580A > G | c.A580G | T194A |
| c.580A > T or | c.A580T or | T194S |
| c.581C > G | c.C581G | |
| c.581C > A | c.C581A | T194N |
| c.581C > T | c.C581T | T194I |
| c.583G > A | c.G583A | G195S |
| c.583G > C | c.G583C | G195R |
| c.583G > T | c.G583T | G195C |
| c.584G > T | c.G584T | G195V |
| c.586A > G | c.A586G | R196G |
| c.587G > A | c.G587A | R196K |
| c.587G > C | c.G587C | R196T |
| c.587G > T | c.G587T | R196I |
| c.589A > G | c.A589G | S197G |
| c.589A > T | c.A589T | S197C |
| c.590G > A | c.G590A | S197N |
| c.590G > C | c.G590C | S197T |
| c.590G > T | c.G590T | S197I |
| c.593T > C | c.T593C | I198T |
| c.593T > G | c.T593G | I198S |
| c.594T > G | c.T594G | I198M |
| c.595G > A | c.G595A | V199M |
| c.595G > C | c.G595C | V199L |
| c.596T > A | c.T596A | V199E |
| c.596T > C | c.T596C | V199A |
| c.596T > G | c.T596G | V199G |
| c.598T > A | c.T598A | Y200N |
| c.599A > C | c.A599C | Y200S |
| c.599A > G | c.A599G | Y200C |
| c.601T > A | c.T601A | S201T |
| c.601T > G | c.T601G | S201A |
| c.602C > A | c.C602A | S201Y |
| c.602C > G | c.C602G | S201C |
| c.602C > T | c.C602T | S201F |
| c.607G > C | c.G607C | E203Q |
| c.608A > C | c.A608C | E203A |
| c.608A > G | c.A608G | E203G |
| c.608A > T | c.A608T | E203V |
| c.609G > C or | c.G609C or | E203D |
| c.609G > T | c.G609T | |
| c.610T > G | c.T610G | W204G |
| c.611G > C | c.G611C | W204S |
| c.611G > T | c.G611T | W204L |
| c.613C > A | c.C613A | P205T |
| c.613C > T | c.C613T | P205S |
| c.614C > T | c.C614T | P205L |
| c.616C > A | c.C616A | L206I |
| c.616C > G | c.C616G | L206V |
| c.616C > T | c.C616T | L206F |
| c.617T > A | c.T617A | L206H |
| c.617T > G | c.T617G | L206R |
| c.619T > C | c.T619C | Y207H |
| c.620A > C | c.A620C | Y207S |
| c.620A > T | c.A620T | Y207F |
| c.623T > A | c.T623A | M208K |
| c.623T > G | c.T623G | M208R |
| c.625T > A | c.T625A | W209R |
| c.625T > G | c.T625G | W209G |
| c.627G > C | c.G627C | W209C |
| c.628C > A | c.C628A | P210T |
| c.628C > T | c.C628T | P210S |
| c.629C > A | c.C629A | P210H |
| c.629C > T | c.C629T | P210L |
| c.631T > C | c.T631C | F211L |
| c.631T > G | c.T631G | F211V |
| c.632T > A | c.T632A | F211Y |
| c.632T > C | c.T632C | F211S |
| c.632T > G | c.T632G | F211C |
| c.635A > C | c.A635C | Q212P |
| c.636A > T | c.A636T | Q212H |
| c.637A > C | c.A637C | K213Q |
| c.637A > G | c.A637G | K213E |
| c.638A > G | c.A638G | K213R |
| c.638A > T | c.A638T | K213M |
| c.640C > A | c.C640A | P214T |
| c.640C > G | c.C640G | P214A |
| c.640C > T | c.C640T | P214S |
| c.641C > A | c.C641A | P214H |
| c.641C > G | c.C641G | P214R |
| c.641C > T | c.C641T | P214L |
| c.643A > C | c.A643C | N215H |
| c.643A > G | c.A643G | N215D |
| c.643A > T | c.A643T | N215Y |
| c.644A > C | c.A644C | N215T |
| c.644A > G | c.A644G | N215S |
| c.[644A > G; | c.A644G/G937T | N215S/D313Y |
| 937G > T] | | |
| c.644A > T | c.A644T | N215I |
| c.645T > A | c.T645A | N215K |
| c.646T > A | c.T646A | Y216N |
| c.646T > C | c.T646C | Y216H |
| c.646T > G | c.T646G | Y216D |
| c.647A > C | c.A647C | Y216S |
| c.647A > G | c.A647G | Y216C |
| c.647A > T | c.A647T | Y216F |
| c.649A > C | c.A649C | T217P |
| c.649A > G | c.A649G | T217A |
| c.649A > T | c.A649T | T217S |
| c.650C > A | c.C650A | T217K |
| c.650C > G | c.C650G | T217R |
| c.650C > T | c.C650T | T217I |
| c.652G > A | c.G652A | E218K |
| c.652G > C | c.G652C | E218Q |
| c.653A > C | c.A653C | E218A |
| c.653A > G | c.A653G | E218G |
| c.653A > T | c.A653T | E218V |
| c.654A > T | c.A654T | E218D |
| c.655A > C | c.A655C | I219L |
| c.655A > T | c.A655T | I219F |
| c.656T > A | c.T656A | I219N |
| c.656T > C | c.T656C | I219T |
| c.656T > G | c.T656G | I219S |
| c.657C > G | c.C657G | I219M |
| c.659G > A | c.G659A | R220Q |
| c.659G > C | c.G659C | R220P |
| c.659G > T | c.G659T | R220L |
| c.661C > A | c.C661A | Q221K |
| c.661C > G | c.C661G | Q221E |
| c.662A > C | c.A662C | Q221P |
| c.662A > G | c.A662G | Q221R |
| c.662A > T | c.A662T | Q221L |
| c.663G > C | c.G663C | Q221H |
| c.664T > A | c.T664A | Y222N |
| c.664T > C | c.T664C | Y222H |
| c.664T > G | c.T664G | Y222D |
| c.665A > C | c.A665C | Y222S |
| c.665A > G | c.A665G | Y222C |
| c.670A > C | c.A670C | N224H |
| c.671A > C | c.A671C | N224T |
| c.671A > G | c.A671G | N224S |
| c.673C > G | c.C673G | H225D |
| c.679C > G | c.C679G | R227G |
| c.682A > C | c.A682C | N228H |
| c.682A > G | c.A682G | N228D |
| c.683A > C | c.A683C | N228T |
| c.683A > G | c.A683G | N228S |
| c.683A > T | c.A683T | N228I |
| c.685T > A | c.T685A | F229I |
| c.686T > A | c.T686A | F229Y |
| c.686T > C | c.T686C | F229S |
| c.687T > A or | c.T687A or | F229L |
| c.687T > G | c.T687G | |
| c.688G > C | c.G688C | A230P |
| c.689C > A | c.C689A | A230D |
| c.689C > G | c.C689G | A230G |
| c.689C > T | c.C689T | A230V |
| c.694A > C | c.A694C | I232L |
| c.694A > G | c.A694G | I232V |
| c.695T > C | c.T695C | I232T |
| c.696T > G | c.T696G | I232M |
| c.698A > C | c.A698C | D233A |
| c.698A > G | c.A698G | D233G |
| c.698A > T | c.A698T | D233V |
| c.699T > A | c.T699A | D233E |
| c.703T > A | c.T703A | S235T |
| c.703T > G | c.T703G | S235A |
| c.710A > T | c.A710T | K237I |
| c.712A > G | c.A712G | S238G |
| c.712A > T | c.A712T | S238C |
| c.713G > A | c.G713A | S238N |
| c.713G > C | c.G713C | S238T |
| c.713G > T | c.G713T | S238I |
| c.715A > T | c.A715T | I239L |
| c.716T > C | c.T716C | I239T |
| c.717A > G | c.A717G | I239M |
| c.718A > G | c.A718G | K240E |
| c.719A > G | c.A719G | K240R |
| c.719A > T | c.A719T | K240M |
| c.720G > C or | c.G720C or | K240N |
| c.720G > T | c.G720T | |
| c.721A > T | c.A721T | S241C |
| c.722G > C | c.G722C | S241T |
| c.722G > T | c.G722T | S241I |
| c.724A > C | c.A724C | I242L |
| c.724A > G | c.A724G | I242V |
| c.724A > T | c.A724T | I242F |
| c.725T > A | c.T725A | I242N |
| c.725T > C | c.T725C | I242T |
| c.725T > G | c.T725G | I242S |
| c.726C > G | c.C726G | I242M |
| c.727T > A | c.T727A | L243M |
| c.727T > G | c.T727G | L243V |
| c.728T > C | c.T728C | L243S |
| c.728T > G | c.T728G | L243W |
| c.729G > C or | c.G729C or | L243F |
| c.729G > T | c.G729T | |
| c.730G > A | c.G730A | D244N |
| c.730G > C | c.G730C | D244H |
| c.730G > T | c.G730T | D244Y |
| c.731A > C | c.A731C | D244A |
| c.731A > G | c.A731G | D244G |
| c.731A > T | c.A731T | D244V |
| c.732C > G | c.C732G | D244E |
| c.733T > G | c.T733G | W245G |
| c.735G > C | c.G735C | W245C |
| c.736A > G | c.A736G | T246A |
| c.737C > A | c.C737A | T246K |
| c.737C > G | c.C737G | T246R |
| c.737C > T | c.C737T | T246I |
| c.739T > A | c.T739A | S247T |
| c.739T > G | c.T739G | S247A |
| c.740C > A | c.C740A | S247Y |
| c.740C > G | c.C740G | S247C |
| c.740C > T | c.C740T | S247F |
| c.742T > G | c.T742G | F248V |
| c.743T > A | c.T743A | F248Y |
| c.743T > G | c.T743G | F248C |
| c.744T > A | c.T744A | F248L |
| c.745A > C | c.A745C | N249H |
| c.745A > G | c.A745G | N249D |
| c.745A > T | c.A745T | N249Y |
| c.746A > C | c.A746C | N249T |
| c.746A > G | c.A746G | N249S |
| c.746A > T | c.A746T | N249I |
| c.747C > G or | c.C747G or | N249K |
| c.747C > A | c.C747A | |
| c.748C > A | c.C748A | Q250K |
| c.748C > G | c.C748G | Q250E |
| c.749A > C | c.A749C | Q250P |
| c.749A > G | c.A749G | Q250R |
| c.749A > T | c.A749T | Q250L |
| c.750G > C | c.G750C | Q250H |
| c.751G > A | c.G751A | E251K |
| c.751G > C | c.G751C | E251Q |
| c.752A > G | c.A752G | E251G |
| c.752A > T | c.A752T | E251V |
| c.754A > G | c.A754G | R252G |
| c.757A > G | c.A757G | I253V |
| c.757A > T | c.A757T | I253F |
| c.758T > A | c.T758A | I253N |
| c.758T > C | c.T758C | I253T |
| c.758T > G | c.T758G | I253S |
| c.760-762delGTT or | c.760_762delGTT or | p.V254del |
| c.761-763del | c.761_763del | |
| c.760G > T | c.G760T | V254F |
| c.761T > A | c.T761A | V254D |
| c.761T > C | c.T761C | V254A |
| c.761T > G | c.T761G | V254G |
| c.763G > A | c.G763A | D255N |
| c.763G > C | c.G763C | D255H |
| c.763G > T | c.G763T | D255Y |
| c.764A > C | c.A764C | D255A |
| c.764A > T | c.A764T | D255V |
| c.765T > A | c.T765A | D255E |
| c.766G > C | c.G766C | V256L |
| c.767T > A | c.T767A | V256D |
| c.767T > G | c.T767G | V256G |
| c.769G > A | c.G769A | A257T |
| c.769G > C | c.G769C | A257P |
| c.769G > T | c.G769T | A257S |
| c.770C > G | c.C770G | A257G |
| c.770C > T | c.C770T | A257V |
| c.772G > C or | c.G772C or | G258R |
| c.772G > A | c.G772A | |
| c.773G > A | c.G773A | G258E |
| c.773G > T | c.G773T | G258V |
| c.775C > A | c.C775A | P259T |
| c.775C > G | c.C775G | P259A |
| c.775C > T | c.C775T | P259S |
| c.776C > A | c.C776A | P259Q |
| c.776C > G | c.C776G | P259R |
| c.776C > T | c.C776T | P259L |
| c.778G > T | c.G778T | G260W |
| c.779G > A | c.G779A | G260E |
| c.779G > C | c.G779C | G260A |
| c.781G > A | c.G781A | G261S |
| c.781G > C | c.G781C | G261R |
| c.781G > T | c.G781T | G261C |
| c.782G > C | c.G782C | G261A |
| c.787A > C | c.A787C | N263H |
| c.788A > C | c.A788C | N263T |
| c.788A > G | c.A788G | N263S |
| c.790G > A | c.G790A | D264N |
| c.790G > C | c.G790C | D264H |
| c.790G > T | c.G790T | D264Y |
| c.793C > G | c.C793G | P265A |
| c.794C > A | c.C794A | P265Q |
| c.794C > T | c.C794T | P265L |
| c.799A > G | c.A799G | M267V |
| c.799A > T | c.A799T | M267L |
| c.800T > C | c.T800C | M267T |
| c.802T > A | c.T802A | L268I |
| c.804A > T | c.A804T | L268F |
| c.805G > A | c.G805A | V269M |
| c.805G > C | c.G805C | V269L |
| c.806T > C | c.T806C | V269A |
| c.808A > C | c.A808C | I270L |
| c.808A > G | c.A808G | I270V |
| c.809T > C | c.T809C | I270T |
| c.809T > G | c.T809G | I270S |
| c.810T > G | c.T810G | I270M |
| c.811G > A | c.G811A | G271S |
| c.[811G > A; | c.G811A/G937T | G271S/D313Y |
| 937G > T] | | |
| c.812G > A | c.G812A | G271D |
| c.812G > C | c.G812C | G271A |
| c.814A > G | c.A814G | N272D |
| c.818T > A | c.T818A | F273Y |
| c.823C > A | c.C823A | L275I |
| c.823C > G | c.C823G | L275V |
| c.827G > A | c.G827A | S276N |
| c.827G > C | c.G827C | S276T |
| c.829T > G | c.T829G | W277G |
| c.830G > T | c.G830T | W277L |
| c.831G > T or | c.G831T or | W277C |
| c.831G > C | c.G831C | |
| c.832A > T | c.A832T | N278Y |
| c.833A > T | c.A833T | N278I |
| c.835C > G | c.C835G | Q279E |
| c.838C > A | c.C838A | Q280K |
| c.839A > G | c.A839G | Q280R |
| c.839A > T | c.A839T | Q280L |
| c.840A > T or | c.A840T or | Q280H |
| c.840A > C | c.A840C | |
| c.841G > C | c.G841C | V281L |
| c.842T > A | c.T842A | V281E |
| c.842T > C | c.T842C | V281A |
| c.842T > G | c.T842G | V281G |
| c.844A > G | c.A844G | T282A |
| c.844A > T | c.A844T | T282S |
| c.845C > T | c.C845T | T282I |
| c.847C > G | c.C847G | Q283E |
| c.848A > T | c.A848T | Q283L |
| c.849G > C | c.G849C | Q283H |
| c.850A > G | c.A850G | M284V |
| c.850A > T | c.A850T | M284L |
| c.851T > C | c.T851C | M284T |
| c.852G > C | c.G852C | M284I |
| c.853G > A | c.G853A | A285T |
| c.854C > G | c.C854G | A285G |
| c.854C > T | c.C854T | A285V |
| c.856C > G | c.C856G | L286V |
| c.856C > T | c.C856T | L286F |
| c.857T > A | c.T857A | L286H |
| c.860G > T | c.G860T | W287L |
| c.862G > C | c.G862C | A288P |
| c.862G > T | c.G862T | A288S |
| c.863C > G | c.C863G | A288G |
| c.863C > T | c.C863T | A288V |
| c.865A > C | c.A865C | I289L |
| c.865A > G | c.A865G | I289V |
| c.866T > C | c.T866C | I289T |
| c.866T > G | c.T866G | I289S |
| c.868A > C or | c.A868C or | M290L |
| c.868A > T | c.A868T | |
| c.868A > G | c.A868G | M290V |
| c.869T > C | c.T869C | M290T |
| c.870G > A or | c.G870A or | M290I |
| c.870G > C or | c.G870C or | |
| c.870G > T | c.G870T | |
| c.871G > A | c.G871A | A291T |
| c.871G > T | c.G871T | A291S |
| c.872C > G | c.C872G | A291G |
| c.874G > T | c.G874T | A292S |
| c.875C > G | c.C875G | A292G |
| c.877C > A | c.C877A | P293T |
| c.880T > A | c.T880A | L294I |
| c.880T > G | c.T880G | L294V |
| c.881T > C | c.T881C | L294S |
| c.882A > T | c.A882T | L294F |
| c.883T > A | c.T883A | F295I |
| c.883T > G | c.T883G | F295V |
| c.884T > A | c.T884A | F295Y |
| c.884T > C | c.T884C | F295S |
| c.884T > G | c.T884G | F295C |
| c.886A > G | c.A886G | M296V |
| c.886A > T or | c.A886T or | M296L |
| c.886A > C | c.A886C | |
| c.887T > C | c.T887C | M296T |
| c.888G > A or | c.G888A or | M296I |
| c.888G > T or | c.G888T or | |
| c.888G > C | c.G888C | |
| c.889T > A | c.T889A | S297T |
| c.892A > G | c.A892G | N298D |
| c.893A > C | c.A893C | N298T |
| c.893A > G | c.A893G | N298S |
| c.893A > T | c.A893T | N298I |
| c.895G > A | c.G895A | D299N |
| c.895G > C | c.G895C | D299H |
| c.897C > G or | c.C897G or | D299E |
| c.897C > A | c.C897A | |
| c.898C > A | c.C898A | L300I |
| c.898C > G | c.C898G | L300V |
| c.898C > T | c.C898T | L300F |
| c.899T > C | c.T899C | L300P |
| c.901C > G | c.C901G | R301G |
| c.902G > A | c.G902A | R301Q |
| c.902G > C | c.G902C | R301P |
| c.902G > T | c.G902T | R301L |
| c.904C > A | c.C904A | H302N |
| c.904C > G | c.C904G | H302D |
| c.904C > T | c.C904T | H302Y |
| c.905A > T | c.A905T | H302L |
| c.907A > G | c.A907G | I303V |
| c.907A > T | c.A907T | I303F |
| c.908T > A | c.T908A | I303N |
| c.908T > C | c.T908C | I303T |
| c.908T > G | c.T908G | I303S |
| c.911G > A | c.G911A | S304N |
| c.911G > C | c.G911C | S304T |
| c.911G > T | c.G911T | S304I |
| c.916C > G | c.C916G | Q306E |
| c.917A > C | c.A917C | Q306P |
| c.917A > T | c.A917T | Q306L |
| c.919G > A | c.G919A | A307T |
| c.919G > C | c.G919C | A307P |
| c.919G > T | c.G919T | A307S |
| c.920C > A | c.C920A | A307D |
| c.920C > G | c.C920G | A307G |
| c.920C > T | c.C920T | A307V |
| c.922A > C | c.A922C | K308Q |
| c.922A > G | c.A922G | K308E |
| c.923A > G | c.A923G | K308R |
| c.923A > T | c.A923T | K308I |
| c.924A > T or | c.A924T or | K308N |
| c.924A > C | c.A924C | |
| c.925G > A | c.G925A | A309T |
| c.925G > C | c.G925C | A309P |
| c.926C > A | c.C926A | A309D |
| c.926C > T | c.C926T | A309V |
| c.928C > A | c.C928A | L310I |
| c.928C > G | c.C928G | L310V |
| c.928C > T | c.C928T | L310F |
| c.931C > A | c.C931A | L311I |
| c.931C > G | c.C931G | L311V |
| c.934C > A | c.C934A | Q312K |
| c.934C > G | c.C934G | Q312E |
| c.935A > G | c.A935G | Q312R |
| c.935A > T | c.A935T | Q312L |
| c.936G > T or | c.G936T or | Q312H |
| c.936G > C | c.G936C | |
| c.937G > T | c.G937T | D313Y |
| c.[937G > T; | c.G937T/G1232A | D313Y/G411D |
| 1232G > A] | | |
| c.938A > G | c.A938G | D313G |
| c.938A > T | c.A938T | D313V |
| c.939T > A | c.T939A | D313E |
| c.940A > G | c.A940G | K314E |
| c.941A > C | c.A941C | K314T |
| c.941A > T | c.A941T | K314M |
| c.942G > C | c.G942C | K314N |
| c.943G > A | c.G943A | D315N |
| c.943G > C | c.G943C | D315H |
| c.943G > T | c.G943T | D315Y |
| c.944A > C | c.A944C | D315A |
| c.944A > G | c.A944G | D315G |
| c.944A > T | c.A944T | D315V |
| c.946G > A | c.G946A | V316I |
| c.946G > C | c.G946C | V316L |
| c.947T > C | c.T947C | V316A |
| c.947T > G | c.T947G | V316G |
| c.949A > C | c.A949C | I317L |
| c.949A > G | c.A949G | I317V |
| c.950T > C | c.T950C | I317T |
| c.951T > G | c.T951G | I317M |
| c.952G > A | c.G952A | A318T |
| c.952G > C | c.G952C | A318P |
| c.953C > A | c.C953A | A318D |
| c.953C > T | c.C953T | A318V |
| c.955A > T | c.A955T | I319F |
| c.956T > C | c.T956C | I319T |
| c.957C > G | c.C957G | I319M |
| c.958A > C | c.A958C | N320H |
| c.959A > C | c.A959C | N320T |
| c.959A > G | c.A959G | N320S |
| c.959A > T | c.A959T | N320I |
| c.961C > A | c.C961A | Q321K |
| c.962A > G | c.A962G | Q321R |
| c.962A > T | c.A962T | Q321L |
| c.963G > C or | c.G963C or | Q321H |
| c.963G > T | c.G963T | |
| c.964G > A | c.G964A | D322N |
| c.964G > C | c.G964C | D322H |
| c.965A > C | c.A965C | D322A |
| c.965A > T | c.A965T | D322V |
| c.966C > A or | c.C966A or | D322E |
| c.966C > G | c.C966G | |
| c.967C > A | c.C967A | P323T |
| c.968C > G | c.C968G | P323R |
| c.970T > G | c.T970G | L324V |
| c.971T > G | c.T971G | L324W |
| c.973G > A | c.G973A | G325S |
| c.973G > C | c.G973C | G325R |
| c.973G > T | c.G973T | G325C |
| c.974G > C | c.G974C | G325A |
| c.974G > T | c.G974T | G325V |
| c.976A > C | c.A976C | K326Q |
| c.976A > G | c.A976G | K326E |
| c.977A > C | c.A977C | K326T |
| c.977A > G | c.A977G | K326R |
| c.977A > T | c.A977T | K326M |
| c.978G > C or | c.G978C or | K326N |
| c.978G > T | c.G978T | |
| c.979C > G | c.C979G | Q327E |
| c.980A > C | c.A980C | Q327P |
| c.980A > T | c.A980T | Q327L |
| c.981A > T | c.A981T | Q327H |
| c.983G > C | c.G983C | G328A |
| c.985T > A | c.T985A | Y329N |
| c.985T > C | c.T985C | Y329H |
| c.985T > G | c.T985G | Y329D |
| c.986A > G | c.A986G | Y329C |
| c.986A > T | c.A986T | Y329F |
| c.988C > A | c.C988A | Q330K |
| c.988C > G | c.C988G | Q330E |
| c.989A > C | c.A989C | Q330P |
| c.989A > G | c.A989G | Q330R |
| c.990G > C | c.G990C | Q330H |
| c.991C > G | c.C991G | L331V |
| c.992T > A | c.T992A | L331H |
| c.992T > C | c.T992C | L331P |
| c.992T > G | c.T992G | L331R |
| c.994A > G | c.A994G | R332G |
| c.995G > C | c.G995C | R332T |
| c.995G > T | c.G995T | R332I |
| c.996A > T | c.A996T | R332S |
| c.997C > G | c.C997G | Q333E |
| c.998A > C | c.A998C | Q333P |
| c.998A > T | c.A998T | Q333L |
| c.1000G > C | c.G1000C | G334R |
| c.1001G > A | c.G1001A | G334E |
| c.1001G > T | c.G1001T | G334V |
| c.1003G > T | c.G1003T | D335Y |
| c.1004A > C | c.A1004C | D335A |
| c.1004A > G | c.A1004G | D335G |
| c.1004A > T | c.A1004T | D335V |
| c.1005C > G | c.C1005G | D335E |
| c.1006A > G | c.A1006G | N336D |
| c.1006A > T | c.A1006T | N336Y |
| c.1007A > C | c.A1007C | N336T |
| c.1007A > G | c.A1007G | N336S |
| c.1007A > T | c.A1007T | N336I |
| c.1009T > G | c.T1009G | F337V |
| c.1010T > A | c.T1010A | F337Y |
| c.1010T > C | c.T1010C | F337S |
| c.1010T > G | c.T1010G | F337C |
| c.1011T > A | c.T1011A | F337L |
| c.1012G > A | c.G1012A | E338K |
| c.1013A > C | c.A1013C | E338A |
| c.1013A > G | c.A1013G | E338G |
| c.1013A > T | c.A1013T | E338V |
| c.1014A > T | c.A1014T | E338D |
| c.1015G > A | c.G1015A | V339M |
| c.1016T > A | c.T1016A | V339E |
| c.1016T > C | c.T1016C | V339A |
| c.1021G > C | c.G1021C | E341Q |
| c.1022A > C | c.A1022C | E341A |
| c.1027C > A | c.C1027A | P343T |
| c.1027C > G | c.C1027G | P343A |
| c.1027C > T | c.C1027T | P343S |
| c.1028C > T | c.C1028T | P343L |
| c.1030C > G | c.C1030G | L344V |
| c.1030C > T | c.C1030T | L344F |
| c.1031T > G | c.T1031G | L344R |
| c.1033T > C | c.T1033C | S345P |
| c.1036G > T | c.G1036T | G346C |
| c.1037G > A | c.G1037A | G346D |
| c.1037G > C | c.G1037C | G346A |
| c.1037G > T | c.G1037T | G346V |
| c.1039T > A | c.T1039A | L347I |
| c.1043C > A | c.C1043A | A348D |
| c.1046G > C | c.G1046C | W349S |
| c.1046G > T | c.G1046T | W349L |
| c.1047G > C | c.G1047C | W349C |
| c.1048G > A | c.G1048A | A350T |
| c.1048G > T | c.G1048T | A350S |
| c.1049C > G | c.C1049G | A350G |
| c.1049C > T | c.C1049T | A350V |
| c.1052T > A | c.T1052A | V351E |
| c.1052T > C | c.T1052C | V351A |
| c.1054G > A | c.G1054A | A352T |
| c.1054G > T | c.G1054T | A352S |
| c.1055C > G | c.C1055G | A352G |
| c.1055C > T | c.C1055T | A352V |
| c.1057A > T | c.A1057T | M353L |
| c.1058T > A | c.T1058A | M353K |
| c.1058T > C | c.T1058C | M353T |
| c.1061T > A | c.T1061A | I354K |
| c.1061T > G | c.T1061G | I354R |
| c.1063A > C | c.A1063C | N355H |
| c.1063A > G | c.A1063G | N355D |
| c.1063A > T | c.A1063T | N355Y |
| c.1064A > G | c.A1064G | N355S |
| c.1066C > G | c.C1066G | R356G |
| c.1066C > T | c.C1066T | R356W |
| c.1067G > A | c.G1067A | R356Q |
| c.1067G > C | c.G1067C | R356P |
| c.1067G > T | c.G1067T | R356L |
| c.1069C > G | c.C1069G | Q357E |
| c.1072G > C | c.G1072C | E358Q |
| c.1073A > C | c.A1073C | E358A |
| c.1073A > G | c.A1073G | E358G |
| c.1074G > T or | c.G1074T or | E358D |
| c.1074G > C | c.G1074C | |
| c.1075A > C | c.A1075C | I359L |
| c.1075A > G | c.A1075G | I359V |
| c.1075A > T | c.A1075T | I359F |
| c.1076T > A | c.T1076A | I359N |
| c.1076T > C | c.T1076C | I359T |
| c.1076T > G | c.T1076G | I359S |
| c.1078G > A | c.G1078A | G360S |
| c.1078G > C | c.G1078C | G360R |
| c.1078G > T | c.G1078T | G360C |
| c.1079G > A | c.G1079A | G360D |
| c.1079G > C | c.G1079C | G360A |
| c.1082G > A | c.G1082A | G361E |
| c.1082G > C | c.G1082C | G361A |
| c.1084C > A | c.C1084A | P362T |
| c.1084C > G | c.C1084G | P362A |
| c.1084C > T | c.C1084T | P362S |
| c.1085C > A | c.C1085A | P362H |
| c.1085C > G | c.C1085G | P362R |
| c.1085C > T | c.C1085T | P362L |
| c.1087C > A | c.C1087A | R363S |
| c.1087C > G | c.C1087G | R363G |
| c.1087C > T | c.C1087T | R363C |
| c.1088G > A | c.G1088A | R363H |
| c.1088G > T | c.G1088T | R363L |
| c.1090T > C | c.T1090C | S364P |
| c.1091C > G | c.C1091G | S364C |
| c.1093T > A | c.T1093A | Y365N |
| c.1093T > G | c.T1093G | Y365D |
| c.1094A > C | c.A1094C | Y365S |
| c.1094A > T | c.A1094T | Y365F |
| c.1096A > C | c.A1096C | T366P |
| c.1096A > T | c.A1096T | T366S |
| c.1097C > A | c.C1097A | T366N |
| c.1097C > T | c.C1097T | T366I |
| c.1099A > C | c.A1099C | I367L |
| c.1099A > T | c.A1099T | I367F |
| c.1101C > G | c.C1101G | I367M |
| c.1102G > A | c.G1102A | A368T |
| c.1102G > C | c.G1102C | A368P |
| c.1103C > G | c.C1103G | A368G |
| c.1105G > A | c.G1105A | V369I |
| c.1105G > C | c.G1105C | V369L |
| c.1105G > T | c.G1105T | V369F |
| c.1106T > C | c.T1106C | V369A |
| c.1106T > G | c.T1106G | V369G |
| c.1108G > A | c.G1108A | A370T |
| c.1108G > C | c.G1108C | A370P |
| c.1109C > A | c.C1109A | A370D |
| c.1109C > G | c.C1109G | A370G |
| c.1109C > T | c.C1109T | A370V |
| c.1111T > A | c.T1111A | S371T |
| c.1112C > G | c.C1112G | S371C |
| c.1117G > A | c.G1117A | G373S |
| c.1117G > T | c.G1117T | G373C |
| c.1118G > C | c.G1118C | G373A |
| c.1120A > G | c.A1120G | K374E |
| c.1121A > C | c.A1121C | K374T |
| c.1121A > G | c.A1121G | K374R |
| c.1121A > T | c.A1121T | K374I |
| c.1123G > C | c.G1123C | G375R |
| c.1124G > A | c.G1124A | G375E |
| c.1124G > C | c.G1124C | G375A |
| c.1126G > A | c.G1126A | V376M |
| c.1126G > C | c.G1126C | V376L |
| c.1127T > A | c.T1127A | V376E |
| c.1127T > G | c.T1127G | V376G |
| c.1129G > A | c.G1129A | A377T |
| c.1129G > C | c.G1129C | A377P |
| c.1129G > T | c.G1129T | A377S |
| c.1130C > G | c.C1130G | A377G |
| c.1135A > G | c.A1135G | N379D |
| c.1136A > C | c.A1136C | N379T |
| c.1136A > T | c.A1136T | N379I |
| c.1137T > A | c.T1137A | N379K |
| c.1138C > A | c.C1138A | P380T |
| c.1138C > G | c.C1138G | P380A |
| c.1139C > A | c.C1139A | P380H |
| c.1139C > G | c.C1139G | P380R |
| c.1139C > T | c.C1139T | P380L |
| c.1142C > A | c.C1142A | A381D |
| c.1147T > A | c.T1147A | F383I |
| c.1148T > A | c.T1148A | F383Y |
| c.1148T > G | c.T1148G | F383C |
| c.1150A > T | c.A1150T | I384F |
| c.1151T > C | c.T1151C | I384T |
| c.1152C > G | c.C1152G | I384M |
| c.1153A > G | c.A1153G | T385A |
| c.1154C > T | c.C1154T | T385I |
| c.1156C > A | c.C1156A | Q386K |
| c.1157A > T | c.A1157T | Q386L |
| c.1158G > C | c.G1158C | Q386H |
| c.1159C > A | c.C1159A | L387I |
| c.1159C > T | c.C1159T | L387F |
| c.1160T > A | c.T1160A | L387H |
| c.1160T > G | c.T1160G | L387R |
| c.1162C > A | c.C1162A | L388I |
| c.1162C > G | c.C1162G | L388V |
| c.1162C > T | c.C1162T | L388F |
| c.1163T > A | c.T1163A | L388H |
| c.1163T > G | c.T1163G | L388R |
| c.1168G > A | c.G1168A | V390M |
| c.1171A > C | c.A1171C | K391Q |
| c.1171A > G | c.A1171G | K391E |
| c.1172A > C | c.A1172C | K391T |
| c.1172A > G | c.A1172G | K391R |
| c.1172A > T | c.A1172T | K391I |
| c.1173A > T | c.A1173T | K391N |
| c.1174A > G | c.A1174G | R392G |
| c.1174A > T | c.A1174T | R392W |
| c.1175G > A | c.G1175A | R392K |
| c.1175G > C | c.G1175C | R392T |
| c.1175G > T | c.G1175T | R392M |
| c.1177A > C | c.A1177C | K393Q |
| c.1177A > G | c.A1177G | K393E |
| c.1178A > C | c.A1178C | K393T |
| c.1179G > C | c.G1179C | K393N |
| c.1180C > A | c.C1180A | L394I |
| c.1181T > A | c.T1181A | L394Q |
| c.1181T > C | c.T1181C | L394P |
| c.1181T > G | c.T1181G | L394R |
| c.1183G > C | c.G1183C | G395R |
| c.1184G > A | c.G1184A | G395E |
| c.1184G > C | c.G1184C | G395A |
| c.1186T > A | c.T1186A | F396I |
| c.1186T > G | c.T1186G | F396V |
| c.1187T > G | c.T1187G | F396C |
| c.1188C > G | c.C1188G | F396L |
| c.1189T > A | c.T1189A | Y397N |
| c.1189T > C | c.T1189C | Y397H |
| c.1190A > C | c.A1190C | Y397S |
| c.1190A > G | c.A1190G | Y397C |
| c.1190A > T | c.A1190T | Y397F |
| c.1192G > A | c.G1192A | E398K |
| c.1192G > C | c.G1192C | E398Q |
| c.1193A > G | c.A1193G | E398G |
| c.1195T > A | c.T1195A | W399R |
| c.1195T > G | c.T1195G | W399G |
| c.1198A > C | c.A1198C | T400P |
| c.1198A > G | c.A1198G | T400A |
| c.1198A > T | c.A1198T | T400S |
| c.1199C > A | c.C1199A | T400N |
| c.1199C > T | c.C1199T | T400I |
| c.1201T > A | c.T1201A | S401T |
| c.1201T > G | c.T1201G | S401A |
| c.1202_{—} | c.1202_{—} | p.T400_{—} |
| 1203insGACTTC | 1203insGACTTC | S401dup |
| c.1202C > T | c.C1202T | S401L |
| c.1204A > G | c.A1204G | R402G |
| c.1204A > T | c.A1204T | R402W |
| c.1205G > C | c.G1205C | R402T |
| c.1205G > T | c.G1205T | R402M |
| c.1206G > C | c.G1206C | R402S |
| c.1207T > G | c.T1207G | L403V |
| c.1208T > C | c.T1208C | L403S |
| c.1209A > T | c.A1209T | L403F |
| c.1210A > G | c.A1210G | R404G |
| c.1211G > A | c.G1211A | R404K |
| c.1211G > C | c.G1211C | R404T |
| c.1211G > T | c.G1211T | R404I |
| c.1212A > T | c.A1212T | R404S |
| c.1213A > G | c.A1213G | S405G |
| c.1216C > G | c.C1216G | H406D |
| c.1217A > T | c.A1217T | H406L |
| c.1218C > G | c.C1218G | H406Q |
| c.1219A > T | c.A1219T | I407L |
| c.1220T > C | c.T1220C | I407T |
| c.1221A > G | c.A1221G | I407M |
| c.1222A > C | c.A1222C | N408H |
| c.1222A > G | c.A1222G | N408D |
| c.1222A > T | c.A1222T | N408Y |
| c.1223A > C | c.A1223C | N408T |
| c.1225C > A | c.C1225A | P409T |
| c.1225C > G | c.C1225G | P409A |
| c.1225C > T | c.C1225T | P409S |
| c.1226C > T | c.C1226T | P409L |
| c.1228A > G | c.A1228G | T410A |
| c.1228A > T | c.A1228T | T410S |
| c.1229C > T | c.C1229T | T410I |
| c.1231G > A | c.G1231A | G411S |
| c.1231G > T | c.G1231T | G411C |
| c.1232G > A | c.G1232A | G411D |
| c.1232G > C | c.G1232C | G411A |
| c.1232G > T | c.G1232T | G411V |
| c.1234A > C | c.A1234C | T412P |
| c.1234A > G | c.A1234G | T412A |
| c.1234A > T | c.A1234T | T412S |
| c.1235C > A | c.C1235A | T412N |
| c.1235C > T | c.C1235T | T412I |
| c.1237G > A | c.G1237A | V413I |
| c.1237G > T | c.G1237T | V413F |
| c.1238T > G | c.T1238G | V413G |
| c.1240T > G | c.T1240G | L414V |
| c.1242G > C | c.G1242C | L414F |
| c.1243C > A | c.C1243A | L415I |
| c.1244T > A | c.T1244A | L415H |
| c.1246C > G | c.C1246G | Q416E |
| c.1247A > T | c.A1247T | Q416L |
| c.1248G > C | c.G1248C | Q416H |
| c.1249C > A | c.C1249A | L417I |
| c.1252G > A | c.G1252A | E418K |
| c.1252G > C | c.G1252C | E418Q |
| c.1253A > C | c.A1253C | E418A |
| c.1253A > G | c.A1253G | E418G |
| c.1254A > T | c.A1254T | E418D |
| c.1255A > G | c.A1255G | N419D |
| c.1255A > T | c.A1255T | N419Y |
| c.1256A > C | c.A1256C | N419T |
| c.1256A > G | c.A1256G | N419S |
| c.1256A > T | c.A1256T | N419I |
| c.1258A > C | c.A1258C | T420P |
| c.1258A > T | c.A1258T | T420S |
| c.1259C > A | c.C1259A | T420K |
| c.1259C > G | c.C1259G | T420R |
| c.1261A > G | c.A1261G | M421V |
| c.1261A > T | c.A1261T | M421L |
| c.1262T > A | c.T1262A | M421K |
| c.1262T > C | c.T1262C | M421T |
| c.1262T > G | c.T1262G | M421R |
| c.1263G > C | c.G1263C | M421I |
| c.1265A > C | c.A1265C | Q422P |
| c.1267A > T | c.A1267T | M423L |
| c.1268T > A | c.T1268A | M423K |
| c.1268T > C | c.T1268C | M423T |
| c.1269G > C | c.G1269C | M423I |
| c.1271C > T | c.C1271T | S424L |
| c.1275A > C | c.A1275C | L425F |
| c.1279G > A | c.G1279A | D427N |
| c.1286T > G | c.T1286G | L429R |

### Kidney Function in Fabry Patients

Progressive decline in renal function is a major complication of Fabry disease. For example, patients associated with a classic Fabry phenotype exhibit progressive renal impairment that can lead to dialysis or renal transplantation.

A frequently used method in the art to assess kidney function is the glomerular filtration rate (GFR). Generally, the GFR is the volume of fluid filtered from the renal glomerular capillaries into the Bowman's capsule per unit time. Clinically, estimates of GFR are made based upon the clearance of creatinine from serum. GFR can be estimated by collecting urine to determine the amount of creatinine that was removed from the blood over a given time interval. Age, body size and gender may also be factored in. The lower the GFR number, the more advanced kidney damage is.

Some studies indicate that untreated Fabry patients experience an average decline in GFR between 7.0 and 18.9 mL/min/1.73 m² per year, while patients receiving an enzyme replacement therapy (ERT) may experience an average decline in GFR between 2.0 and 2.7 mL/min/1.73 m² per year, although more rapid declines may occur in patients with more significant proteinuria or with more severe chronic kidney disease. Thus, even with patients receiving therapy there is a need to determine an appropriate dose of the therapeutic to account for a patient's developing impairment of renal function. Adjustment of the dose can be used to avoid an accumulation of the therapeutic to a level that is outside the therapeutic index or to a level where the patient experiences toxicity.

An estimated GFR (eGFR) is calculated from serum creatinine using an isotope dilution mass spectrometry (IDMS) traceable equation. Two of the most commonly used equations for estimating glomerular filtration rate (GFR) from serum creatinine are the Chronic Kidney Disease Epidemiology Collaboration (CKD-EPI) equation and the Modification of Diet in Renal Disease (MDRD) Study equation. Both the MDRD Study and CKD-EPI equations include variables for age, gender, and race, which may allow providers to observe that CKD is present despite a serum creatinine concentration that appears to fall within or just above the normal reference interval.

The CKD-EPI equation uses a 2-slope “spline” to model the relationship between GFR and serum creatinine, age, sex, and race. CKD-EPI equation expressed as a single equation:

GFR=141×min(S_{cr}/κ,1)α×max(S_{cr}/κ,1)−1.209×0.993^{Age}×1.018 [if female]×1.159 [if black]
where:
S_{cr} is serum creatinine in mg/dL,
κ is 0.7 for females and 0.9 for males,
α is −0.329 for females and −0.411 for males,
min indicates the minimum of S_{cr}/κ or 1, and
max indicates the maximum of S_{cr}/κ or 1.

The following is the IDMS-traceable MDRD Study equation (for creatinine methods calibrated to an IDMS reference method):

$$GFR ( mL / min / 1.73 {m}^{2} ) = 175 \times {( {S}_{cr} )}^{- 1.154} \times {( Age )}^{- 0.203} \times ( 0.742 if female ) \times ( 1.212 if African American )$$

The equation does not require weight or height variables because the results are reported normalized to 1.73 m² body surface area, which is an accepted average adult surface area. The equation has been validated extensively in Caucasian and African American populations between the ages of 18 and 70 with impaired kidney function (eGFR <60 mL/min/1.73 m²) and has shown good performance for patients with all common causes of kidney disease.

One method for estimating the creatinine clearance rate (eC_{cr}) is using the Cockcroft-Gault equation, which in turn estimates GFR in ml/min:

$$Creatinine Clearance ( ml / min ) = [ ( 140 - Age ) \times Mass {( kg )}^{*} ] \div 72 \times Serum Creatinine ( mg / dL )$$

$${[}^{*} multiplied by 0.85 if female ]$$

The Cockcroft-Gault equation is the equation suggested for use by the Food and Drug Administration for renal impairment studies. It is common for the creatinine clearance calculated by the Cockcroft-Gault formula to be normalized for a body surface area of 1.73 m². Therefore, this equation can be expressed as the estimated eGFR in mL/min/1.73 m². The normal range of GFR, adjusted for body surface area, is 100-130 ml/min/1.73 m² in men and 90-120 ml/min/1.73 m² in women younger than the age of 40.

The severity of chronic kidney disease has been defined in six stages (see also Table 2): (Stage 0) Normal kidney function—GFR above 90 mL/min/1.73 m² and no proteinuria; (Stage 1)—GFR above 90 mL/min/1.73 m² with evidence of kidney damage; (Stage 2) (mild)—GFR of 60 to 89 mL/min/1.73 m² with evidence of kidney damage; (Stage 3) (moderate)—GFR of 30 to 59 mL/min/1.73 m²; (Stage 4) (severe)—GFR of 15 to 29 mL/min/1.73 m²; (Stage 5) kidney failure—GFR less than 15 mL/min/1.73 m². Table 2 below shows the various kidney disease stages with corresponding GFR levels.

TABLE 2

| Chronic Kidney | GFR level |
|---|---|
| Disease Stage | (mL/min/1.73 m²) |
| Stage 1 (Normal) | ≥90 |
| Stage 2 (Mild) | 60-89 |
| Stage 3 (Moderate) | 30-59 |
| Stage 4 (Severe) | 15-29 |
| Stage 5 (Kidney Failure) | <15 |

As used herein, the term “severe renal impairment” includes a renal impairment corresponding to both Stage 4 and Stage 5 kidney disease stages provided in Table 2. For example, “severe renal impairment” includes a renal impairment wherein an eGFR level, as measured by the Cockgroft-Gault equation, is less than about 30 mL/min/1.73 m².

### Dosing, Formulation and Administration

One or more of the dosing regimens described herein are particularly suitable for Fabry patients who have some degree of renal impairment. Amicus Therapeutics has sponsored two Phase 3 studies using migalastat 150 mg every other day (QOD) in Fabry patients. FACETS (011, NCT00925301) was a 24-month trial, including a 6-month double-blind, placebo-controlled period, in 67 enzyme replacement therapy (ERT)—can patients. ATTRACT (012, NCT01218659) was an active-controlled, 18-month trial in 57 ERT-experienced patients with a 12-month open-label extension (OLE). Both the FACETS and ATTRACT studies included patients having an estimated glomerular filtration rate (eGFR) of ≥30 ml/min/1.73 m². Accordingly, both studies included Fabry patients with normal renal function as well as patients with mild and moderate renal impairment, but neither study included patients with severe renal impairment.

The Phase 3 studies of migalastat treatment of Fabry patients established that 150 mg every other day slowed the progression of the disease as shown by surrogate markers. However, in some embodiments, the migalastat dosing regimen may be adjusted in some Fabry patients because these patients can experience kidney deterioration. With a slowing in the ability to clear the drug from the body there can be an increasing exposure to the patient to the drug. Thus, in some embodiments a dose adjustment protocol is provided to inform physicians of the best dose taking into consideration the current clearance profile from the body. Dose adjustment is particularly difficult with a chaperone because it is an inhibitor, and a delicate balance must be reached such that the chaperone is present in amounts great enough to be therapeutic, but also not so great that the chaperone inhibits enzyme function (which would exacerbate the disease). As such, it is difficult to predict correct dosing, which is further complicated in patients who have reduced capacity to clear the migalastat.

Accordingly, in one or more embodiments, the Fabry patient with renal impairment is administered from about 50 mg to about 200 mg FBE of migalastat at a frequency of less than once every week. In one or more embodiments, the migalastat or salt thereof is administered at a frequency of once every two weeks (also referred to as “QOW” or “Q14D”).

In various embodiments, the doses described herein pertain to migalastat hydrochloride or an equivalent dose of migalastat or a salt thereof other than the hydrochloride salt. In some embodiments, these doses pertain to the free base of migalastat. In alternate embodiments, these doses pertain to a salt of migalastat. In further embodiments, the salt of migalastat is migalastat hydrochloride. The administration of migalastat or a salt of migalastat is referred to herein as “migalastat therapy”.

The effective amount of migalastat or salt thereof can be in the range from about 50 mg FBE to about 200 mg FBE. Exemplary doses include about 50 mg FBE, about 55 mg FBE, about 60 mg FBE, about 65 mg FBE, about 70 mg FBE, about 75 mg FBE, about 80 mg FBE, about 85 mg FBE, about 90 mg FBE, about 95 mg FBE, about 100 mg FBE, about 105 mg FBE, about 110 mg FBE, about 115 mg FBE, about 120 mg FBE, about 125 mg FBE, about 130 mg FBE, about 135 mg FBE, about 140 mg FBE, about 145 mg FBE, about 150 mg FBE, about 155 mg FBE, about 160 mg FBE, about 165 mg FBE, about 170 mg FBE, about 175 mg FBE, about 180 mg FBE, about 185 mg FBE, about 190 mg FBE, about 195 mg FBE, about 200 mg FBE, about 205 mg FBE, about 210 mg FBE, about 215 mg FBE, about 220 mg FBE, about 225 mg FBE, about 230 mg FBE, about 235 mg FBE, about 240 mg FBE, about 245 mg FBE, or about 250 mg FBE.

Again, it is noted that 150 mg of migalastat hydrochloride is equivalent to 123 mg of the free base form of migalastat. Thus, in one or more embodiments, the dose is 150 mg of migalastat hydrochloride or an equivalent dose of migalastat or a salt thereof other than the hydrochloride salt, administered at a frequency of less than once every week. As set forth above, this dose is referred to as 123 mg FBE of migalastat. In further embodiments, the dose is 150 mg of migalastat hydrochloride administered at a frequency of once every other week. In other embodiments, the dose is 123 mg of the migalastat free base administered at a frequency of once every other week.

In various embodiments, the effective amount is about 61 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, or about 244 mg of migalastat hydrochloride.

Thus, in one or more embodiments, the dose is 150 mg migalastat hydrochloride or an equivalent dose of migalastat or a salt thereof other than the hydrochloride salt, administered at a frequency of less than once every week. In further embodiments, the dose is 150 mg migalastat hydrochloride administered every two weeks. In other embodiments, the dose is 123 mg of migalastat free base administered at a frequency of less than once every week. Longer dosing intervals (e.g. eight to twenty-one days) may be useful with a higher degree of renal impairment compared to a dosing frequency of every other day. Such longer dosing intervals include every eight, ten, fourteen, or twenty-one days.

Accordingly, in one or more embodiments, the Fabry patient with renal impairment is administered from about 50 mg to about 100 mg FBE of migalastat at a frequency of between about once every three days and about once every two weeks. In one or more embodiments, the migalastat or salt thereof is administered at a frequency of once every week (also referred to as “QW” or “Q7D”).

In various embodiments, the doses described herein pertain to migalastat hydrochloride or an equivalent dose of migalastat or a salt thereof other than the hydrochloride salt. In some embodiments, these doses pertain to the free base of migalastat. In alternate embodiments, these doses pertain to a salt of migalastat. In further embodiments, the salt of migalastat is migalastat hydrochloride. The administration of migalastat or a salt of migalastat is referred to herein as “migalastat therapy”.

The effective amount of migalastat or salt thereof can be in the range from about 50 mg FBE to about 100 mg FBE. Exemplary doses include about 50 mg FBE, about 55 mg FBE, about 60 mg FBE, about 65 mg FBE, about 70 mg FBE, about 75 mg FBE, about 80 mg FBE, about 85 mg FBE, about 90 mg FBE, about 95 mg FBE, or about 100 mg FBE migalastat.

Again, it is noted that 100 mg of migalastat hydrochloride is equivalent to 82 mg of the free base form of migalastat. Thus, in one or more embodiments, the dose is 100 mg of migalastat hydrochloride or an equivalent dose of migalastat or a salt thereof other than the hydrochloride salt, administered at a frequency of once every week. As set forth above, this dose is referred to as 82 mg FBE of migalastat. In further embodiments, the dose is 100 mg of migalastat hydrochloride administered at a frequency of once every week. In other embodiments, the dose is 82 mg of the migalastat free base administered at a frequency of once every week.

In various embodiments, the effective amount is about 61 mg, about 65 mg, about 70 mg, about 75 mg, about 80 mg, about 85 mg, about 90 mg, about 95 mg, about 100 mg, about 105 mg, about 110 mg, about 115 mg, about 120 mg, or about 122 mg of migalastat hydrochloride.

Thus, in one or more embodiments, the dose is 100 mg migalastat hydrochloride or an equivalent dose of migalastat or a salt thereof other than the hydrochloride salt, administered at a frequency of between about once every three days and about once every two weeks. For example, the frequency may be once about every 3 days, once about every 4 days, once about every 5 days, once about every 6 days, once about every 7 days, once about every 8 days, once about every 9 days, once about every 10 days, once about every 11 days, once about every 12 days, once about every 13 days, or once about every 14 days. In further embodiments, the dose is 100 mg migalastat hydrochloride administered every week. In other embodiments, the dose is 82 mg of migalastat free base administered every week. Longer dosing intervals (e.g. eight to fourteen days) may be useful with a higher degree of renal impairment compared to a dosing frequency of every other day.

The administration of migalastat may be for a certain period of time. In one or more embodiments, the migalastat is administered for a duration of at least 28 days, such as at least 30, 60 or 90 days or at least 3, 4, 5, 6, 7, 8, 9, 10, 11, 12, 16, 20 or 24 months or at least 1, 2 or 3 years. In various embodiments, the migalastat therapy is long-term migalastat therapy of at least 6 months, such as at least 6, 7, 8, 9, 10, 11, 12, 16, 20 or 24 months or at least 1, 2 or 3 years.

Administration of migalastat according to the present invention may be in a formulation suitable for any route of administration, but is preferably administered in an oral dosage form such as a tablet, capsule or solution. As one example, the patient is orally administered one or more capsules each containing 25 mg, 50 mg, 75 mg, 100 mg or 150 mg migalastat hydrochloride (i.e. 1-deoxygalactonojirimycin hydrochloride) or an equivalent dose of migalastat or a salt thereof other than the hydrochloride salt.

In some embodiments, the PC (e.g., migalastat or salt thereof) is administered orally. In one or more embodiments, the PC (e.g., migalastat or salt thereof) is administered by injection. The PC may be accompanied by a pharmaceutically acceptable carrier, which may depend on the method of administration.

In one embodiment of the invention, the chaperone compound is administered as monotherapy, and can be in a form suitable for any route of administration, including e.g., orally in the form tablets or capsules or liquid, or in sterile aqueous solution for injection. In other embodiments, the PC is provided in a dry lyophilized powder to be added to the formulation of the replacement enzyme during or immediately after reconstitution to prevent enzyme aggregation in vitro prior to administration.

In some embodiments, the chaperone compound is provided during a dialysis treatment, including intravenous delivery through a dialysis machine.

When the chaperone compound is formulated for oral administration, the tablets or capsules can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active chaperone compound.

The pharmaceutical formulations of the chaperone compound suitable for parenteral/injectable use generally include sterile aqueous solutions (where water soluble), or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the purified enzyme and the chaperone compound in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The formulation can contain an excipient. Pharmaceutically acceptable excipients which may be included in the formulation are buffers such as citrate buffer, phosphate buffer, acetate buffer, bicarbonate buffer, amino acids, urea, alcohols, ascorbic acid, and phospholipids; proteins, such as serum albumin, collagen, and gelatin; salts such as EDTA or EGTA, and sodium chloride; liposomes; polyvinylpyrollidone; sugars, such as dextran, mannitol, sorbitol, and glycerol; propylene glycol and polyethylene glycol (e.g., PEG-4000, PEG-6000); glycerol; glycine or other amino acids; and lipids. Buffer systems for use with the formulations include citrate; acetate; bicarbonate; and phosphate buffers. Phosphate buffer is a preferred embodiment.

The route of administration of the chaperone compound may be oral (preferably) or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of the chaperone compound may be by periodic injections of a bolus of the preparation, or may be administered by intravenous or intraperitoneal administration from a reservoir which is external (e.g., an i.v. bag, dialysis machine) or internal (e.g., a bioerodable implant).

Embodiments relating to pharmaceutical formulations and administration may be combined with any of the other embodiments of the invention, for example embodiments relating to a method of treating a patient with Fabry disease, a method of enhancing α-galactosidase A in a patient diagnosed with or suspected of having Fabry disease, use of a pharmacological chaperone for α-galactosidase A for the manufacture of a medicament for treating a patient diagnosed with Fabry disease or to a pharmacological chaperone for α-galactosidase A for use in treating a patient diagnosed with Fabry disease as well as embodiments relating to amenable mutations, the PCs and suitable dosages thereof.

In one or more embodiments, chaperone is administered in combination with enzyme replacement therapy. Enzyme replacement therapy increases the amount of protein by exogenously introducing wild-type or biologically functional enzyme by way of infusion. This therapy has been developed for many genetic disorders, including lysosomal storage disorders such as Fabry disease, as referenced above. After the infusion, the exogenous enzyme is expected to be taken up by tissues through non-specific or receptor-specific mechanism. In general, the uptake efficiency is not high, and the circulation time of the exogenous protein is short. In addition, the exogenous protein is unstable and subject to rapid intracellular degradation as well as having the potential for adverse immunological reactions with subsequent treatments. In one or more embodiments, the chaperone is administered at the same time as replacement enzyme. In some embodiments, the chaperone is co-formulated with the replacement enzyme.

In one or more embodiments, a patient is switched from enzyme replace therapy (ERT) to migalastat therapy. In some embodiments, a patient on ERT is identified, the patient's ERT is discontinued, and the patient begins receiving migalastat therapy. The migalastat therapy can be in accordance with any of the methods described herein. In various embodiments, the patient has some degree of renal impairment, such as mild, moderate or severe renal impairment.

### Monitoring Lyso-Gb3 and Migalastat Levels

Lyso-Gb3 (globotriaosylsphingosine) can be monitored to determine whether substrate is being cleared from the body of a Fabry patient. Higher levels of lyso-Gb3 correlate with higher levels of substrate. If a patient is being successfully treated, then lyso-Gb3 levels are expected to drop.

Over time, the levels of lyso-Gb3 may rise which can be due to either disease progression and/or decreasing ability of the kidneys to clear migalastat from the patient's body. Lyso-Gb3 levels will rise when the level of migalastat is too high because at higher levels the migalastat acts as an inhibitor of α-Gal A, thus preventing the enzyme from binding to the target substrate. Individuals with normal kidney function will generally clear a 150 mg dose of migalastat hydrochloride by 48 hours (i.e., C_{48 h} to below a level of quantification of about 5 ng/ml). In cases of severe kidney impairment, C_{48 h} may be 250 or even above 300 ng/ml. It is thought that high levels of migalastat are due to impaired kidney function because migalastat does not have other known interactions that would otherwise result in high levels.

Accordingly, another aspect of the invention pertains to methods for treatment of Fabry disease in a patient having renal impairment. In one or more embodiments, the method comprises administering to the patient about 50 mg to about 200 mg FBE of migalastat or salt thereof at a first frequency of less than once every week for a first time period; and administering to the patient about 50 mg to about 200 mg FBE of migalastat or salt thereof at a longer dosing interval for a second time period. In some embodiments, the dosing frequency is adjusted after measuring lyso-Gb3 and/or migalastat levels. In some embodiments, the dosing frequency is adjusted after a change in the patient's kidney function (e.g. eGFR). For example, the dosing frequency can be adjusted as the patient's eGFR indicates a change from mild renal impairment to moderate renal impairment or a change from moderate renal impairment to severe renal impairment.

Accordingly, another aspect of the invention pertains to methods for treatment of Fabry disease in a patient having renal impairment. In one or more embodiments, the method comprises administering to the patient about 50 mg to about 100 mg FBE of migalastat or salt thereof at a first frequency of between every three days and two weeks for a first time period; and administering to the patient about 50 mg to about 100 mg FBE of migalastat or salt thereof at a longer dosing interval for a second time period. In some embodiments, the dosing frequency is adjusted after measuring lyso-Gb3 and/or migalastat levels. In some embodiments, the dosing frequency is adjusted after a change in the patient's kidney function (e.g. eGFR). For example, the dosing frequency can be adjusted as the patient's eGFR indicates a change from mild renal impairment to moderate renal impairment or a change from moderate renal impairment to severe renal impairment.

In some embodiments, the migalastat or salt thereof is administered at a first frequency for a first time period, and then administered at a second frequency for a second time period. The first frequency is greater (i.e., more frequent) than the second frequency. The first frequency and the second frequency may be any dosing interval disclosed herein. In some embodiments, the migalastat or salt thereof is administered at a first frequency for a first time period, then administered at a second frequency for a second time period, and then administered at a third frequency for a third time period. The first frequency is greater (i.e., more frequent) than the second frequency, and the second frequency is greater than the third frequency.

In some embodiments, the dosing frequency is adjusted in response to a reduction in the patient's eGFR. In exemplary embodiments, when the patient's eGFR is reduced below 30 mL/min/1.73 m², the dosing frequency can be adjusted from every week to every 10 days, or every 2 weeks, or every 3 weeks, or every 4 weeks. Or it can be adjusted from once every 2 weeks to once every 3 weeks, or every 4 weeks, or every 5 weeks. In exemplary embodiments, when the patient's eGFR is reduced below 20 mL/min/1.73 m², the dosing frequency can be adjusted from every week to every 10 days, or every 2 weeks, or every 3 weeks, or every 4 weeks. Or it can be adjusted from once every 2 weeks to once every 3 weeks, or every 4 weeks, or every 5 weeks. Other adjustments in dosing frequency can be made from one dosing interval to a longer dosing interval as described above. In some embodiments, the patient suffers from severe renal impairment.

In some embodiments, the method further comprises measuring migalastat levels. In one or more embodiments, migalastat concentration (e.g., ng/ml) is measured. In some embodiments, the total area under the curve (AUC_{0-∞}) is measured. In one or more embodiments, the lowest concentration the migalastat reaches before the next dose (C_{trough}) is measured. C_{trough} for Q3D will be the concentration at 72 hours (C₇₂). C_{trough} for Q7D will be the concentration at 168 hours (C₁₆₈). Similarly, C_{trough} for Q14D will be the concentration at 336 hours (C₃₃₆). In one or more embodiments, the targeted C_{trough} values are at or near below the level of quantitation (BLQ). Such C_{trough} values indicate that the migalastat is being cleared from the body at an appropriate rate (i.e., is almost completely cleared before administration of the next dose).

Migalastat levels can be measured via methods known in the art. For example, if measuring migalastat from tissue samples, tissue aliquots may be homogenized (7 μL water per 1 mg tissue) using a homogenizer (e.g., FastPrep-24 from MP Biomedical, Irvine, CA). Microcentrifuge tubes containing 100 μl of the tissue homogenate or 50 μl of plasma may then be spiked with 500 ng/ml 13C d2-AT1001 HCl internal standard (manufactured by MDS Pharma Services). A 600 μl volume of 5 mM HCl in 95/5 MeOH:H₂O can then be added and the tubes vortexed for 2 minutes, followed by centrifugation at 21000×g for 10 minutes at room temperature. The supernatants may then be collected into a clean, 96-well plate, diluted with 5 mM HCl in dH₂O and applied to a 96-well solid phase extraction (SPE) plate (Waters Corp., Milford MA). After several wash steps and elution into a clean, 96-well plate, the extracts may be dried down under N₂ and reconstituted with mobile phase A. Migalastat levels can then be determined by liquid chromatography-tandem mass spectroscopy (LC-MS/MS) (e.g., LC: Shimadzu; MS/MS: ABSciex API 5500 MS/MS). The liquid chromatography can be conducted using an CAN:water:formate binary mobile phase system (mobile phase A: 5 mM ammonium formate, 0.5% formic acid in 95:5 CAN:water; mobile phase B: 5 mM ammonium formate, 0.5% formic acid in 5:47.5:47.5 CAN:MeOH:water) with a flow rate of 0.7 mL/minute on an Halo HILIC column (150×4.6 mm, 2.7 μm) (Advanced Materials Technology, Inc.). MS/MS analysis may be carried out under APCi positive ion mode. The same procedure may be followed for migalastat determination in plasma except without homogenization. The following precursor ion→product ion transitions may be monitored: mass/charge (m/z) 164.1→m/z 80.1 for migalastat and m/z 167.1→m/z 83.1 for the internal standard. A 12-point calibration curve and quality control samples may be prepared. The ratio of the area under the curve for migalastat to that of the internal standard is then determined and final concentrations of migalastat in each sample calculated using the linear least squares fit equation applied to the calibration curve. To derive approximate molar concentrations, one gram of tissue may be estimated as one mL of volume.

Migalastat concentration can be measured from plasma samples at various times to monitor clearance from the body. A clinically relevant increase in C_{trough} suggests significant accumulation of plasma migalastat concentration. If the migalastat is not cleared from the body enough prior to the next dose administration, then the levels of migalastat can build up, possibly leading to an inhibitory effect. Thus, in one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in C_{trough} compared to normal renal function C_{trough}. In one or more embodiments, the C_{trough} of normal renal function at is BLQ. In some embodiments, BLQ is 5 ng/ml of migalastat. A person with normal kidney function will generally clear 150 mg of migalastat HCl in 48 hours. Thus, a patient that is currently on a dosing QOD regimen of 150 mg of migalastat HCl should reach BLQ by 48 hours, which is also the C_{trough} value. If values above BLQ are measured at 48 hours in a patient on a QOD dosing regimen, then this may indicate a need to change the dosing interval. Accordingly, in one or more embodiments, the C_{trough} value of a patient with renal impairment (C_{48 h} if on a QOD regimen, C₉₆ if on a Q4D regimen or C₁₆₈ if on a Q7D regimen) will be compared with C_{trough} of a person with normal renal function (C_{48 h}).

In one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in AUC_{0-∞}, compared to normal renal function AUC_{0-∞}.

In some embodiments, samples may be taken at 0, 1, 2, 3, 4, 6, 8, 12, 24, 48, 72, 96, 120, 144 and/or 168 hours after administration. In some embodiments, the migalastat concentration 48 hours after administration is measured. In some embodiments, the administration of the second time period is begun after more than about 5, 10, 15, 20, 25, 40, 50, 60, 70, 80, 90, 100, 125, 150, 175 or 200 ng/mL of migalastat is measured 48 hours after administration of the migalastat during the first time period is measured.

In further embodiments, the method further comprises measuring lyso-Gb3 in one or more plasma samples from the patient. A first baseline lyso-Gb3 level may be determined during the first time period. As used herein, “baseline lyso-Gb3 level” refers to the lowest plasma lyso-Gb3 value measured during a given time period or dosing regimen. Thus, if the lyso-Gb3 levels go up significantly from the baseline lyso-Gb3 levels, this may indicate kidney disease progression and/or improper clearance of migalastat. Thus, in further embodiments, the administration of the second time period is begun after an increase (e.g., of at least about 20, 25, 30, 33, 35, 40, 45 or 50% and/or 1, 1.25, 1.5, 1.75, 2, 2.25, 2.5 or 3 nM) above the first baseline lyso-Gb3 level is measured. A 33% and/or 2 nM increase from baseline in plasma lyso-Gb3 has been deemed clinically relevant based upon Phase 3 data in Fabry patients signaling either inhibition-induced migalastat exposure from decline in renal function and/or progression of disease condition. Lyso-Gb3 levels may be measured at varying frequencies (e.g., about once every 2, 3, 4 or 5 months). It is thought that it takes about 3 months for a baseline lyso-Gb3 level to be established once a dosing regimen has been started.

Lyso-Gb3 can be measured via methods known in the art using validated assays. As with migalastat, lyso-Gb3 levels may be determined using liquid chromatography-tandem mass spectroscopy (LC-MS/MS) (e.g., LC: Shimadzu; MS/MS: ABSciex API 5500 MS/MS). For example, one process of measuring plasma lyso-Gb3 is described in Hamler, Rick, et al. “Accurate quantitation of plasma globotriaosylsphingosine (lyso-Gb3) in normal individuals and Fabry disease patients by liquid chromatography-tandem mass spectrometry (LC-MS/MS).” Molecular Genetics and Metabolism, Volume 114.2 (2015):S51. In one or more embodiments, lyso-Gb3 is measured in samples from a patient's urine.

Once the dose regimen has been changed, a new plasma lyso-Gb3 baseline level will be established. Any new dose regimen modifications will be based on a comparison to the subject's most current baseline level. For example, a new baseline level may be established as follows: if a subject has a decrease in plasma lyso-Gb3 relative to their previous measurement, a confirmatory retest may take place. If the confirmatory value is also lower than their previous measurement, the average of the 2 values will be the subject's new baseline level. If the retest is NOT lower than the subject's previous measurement, the previous measurement will continue as the current baseline level until the next visit.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in various embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

Patents, patent applications, publications, product descriptions, and protocols are cited throughout this application, the disclosures of which are incorporated herein by reference in their entireties for all purposes.

### EXAMPLES

### Example 1: Pharmacokinetics of Migalastat in Non-Fabry Patients with Renal Impairment

A Phase 1 trial involving six non-Fabry subjects with end stage renal disease (ESRD) receiving dialysis treatment was conducted. The dialysis treatment was hemodialysis (HD) or hemodiafiltration (HDF).

### Study Design

A Phase 1, open-label and non-randomized trial involving six non-Fabry subjects with ESRD receiving dialysis treatment (standard HD and HDF, n=3 each) and six matched controls with normal renal function (NRF) was conducted at a single center. Recruitment started with the ESRD subjects. Matched control subjects with NRF were recruited in parallel, but not treated at the same time; each NRF subject was enrolled after the follow-up visit of the respective ESRD subject.

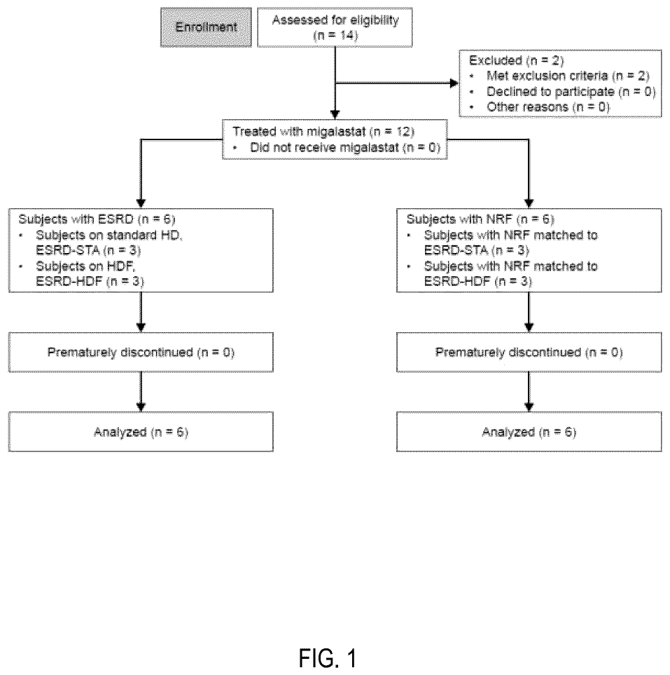

FIG. 1 illustrates a flow diagram of subject disposition. Referring to FIG. 1, the term “STA” means hemodialysis (i.e., “STA” is equivalent to “HD”). The inclusion criteria for ESRD subjects were as follows: eGFR according to the Modification of Diet in Renal Disease equation (eGFR_{MDRD})<15 mL/min/1.73 m²; receives HD or HDF for least 4 hours thrice a week; stable on dialysis regimen for least 2 months; 18 to 79 years of age; body mass index (BMI) within 18.0 to 35.0 kg/m²; systolic blood pressure ≥90 to ≤179 mmHg, diastolic blood pressure ≥55 to ≤100 mmHg measured after 5 minutes in the supine position; resting pulse rate ≥50 to ≤99 beats/min measured after 5 minutes in the supine position; electrocardiogram (ECG) without clinically significant abnormalities; no febrile or infectious illness for at least 7 days prior to the first administration of study drug; willing to provide written informed consent, authorization for use and disclosure of personal health information or research-related health information, or has a legally authorized representative who has given written informed consent; and, if of reproductive potential, both male and female subjects agree to use a medically accepted method of contraception during the study and for at least 30 days after the last dose of study drug.

Exclusion criteria for ESRD subjects included: excess xanthine consumption (>5 cups of coffee or equivalent per day); more than moderate alcohol consumption (>35 g of ethanol regularly per day or >245 g regularly per week); any history of alcohol or drug abuse; more than moderate smoking (>10 cigarettes per day); consumption of xanthine-containing food or beverages within 48 hours before first dosing; consumption of grapefruit-containing food and beverages within 1 week before first dosing; positive urine drug screen; positive alcohol breath test; use of any new medication within 4 weeks before dosing (or at least 10 times the respective elimination half-life, whichever is longer) except hormonal contraceptives, paracetamol, and those drugs the renally impaired subject is currently taking for treatment of renal and/or concomitant disease; any change of chronic medication <14 days prior to first dosing; requirement for treatment with miglitol or miglustat; previous kidney transplantation; any history of allergy or sensitivity to migalastat or other iminosugars or excipients; any history of drug hypersensitivity, asthma, urticaria, or other severe allergic diathesis as well as current hay fever; any intercurrent illness or condition that may preclude the subject from fulfilling the protocol requirements or suggests to the investigator that the potential subject may have an unacceptable risk by participating in this study; any severe or unsuitable concomitant medical condition (cardiovascular, neurological, hepatic, metabolic, hematological, immunological, pulmonary, or gastrointestinal disorder); any clinically significant abnormal laboratory value(s) and clinically significant ECG findings not due to the underlying disease; positive test for human immunodeficiency virus (HIV) antibodies or HIV-1 p24-antigen; positive hepatitis B-virus surface antigen (HBsAg) test; positive anti-hepatitis C-virus antibodies (anti-HCV) test; treatment with an investigational drug within 30 days of study start; inability to comply with study requirements or deemed otherwise unsuitable for study entry in the opinion of the investigator; female subject is pregnant or breastfeeding; blood donation within 30 days before signing informed consent to this study.

Inclusion criteria for healthy subjects matched to the ESRD subjects for age±10 years, body weight±10 kg, and sex, included the following: eGFR_{MDRD} ≥80 mL/min/1.73 m²; 18 to 79 years of age; BMI within 18.0 to 35.0 kg/m²; systolic blood pressure ≥90 to ≤145 mmHg, diastolic blood pressure ≥55 to ≤89 mmHg measured after 5 minutes in the supine position; resting pulse rate ≥50 to ≤99 beats/min measured after 5 minutes in the supine position; ECG without clinically significant abnormalities; no febrile or infectious illness for at least 7 days prior to the first administration of study drug; in general good health as determined by medical and surgical history, physical examination, 12-lead ECG, vital signs, and clinical laboratory evaluations; willing to provide written informed consent and authorization for use and disclosure of personal health information or research-related health information; and, if of reproductive potential, both male and female subjects agree to use a medically accepted method of contraception during the study and for at least 30 days after the last dose of study drug.

Exclusion criteria for healthy subjects included: excess xanthine consumption (>5 cups of coffee or equivalent per day); more than moderate alcohol consumption (>35 g of ethanol regularly per day or >245 g regularly per week); any history of alcohol or drug abuse; more than moderate smoking (>10 cigarettes per day); consumption of xanthine-containing food or beverages within 48 hours before dosing; consumption of grapefruit-containing food and beverages within 1 week before dosing; positive urine drug screen; positive alcohol breath test; any use of medication within 4 weeks before dosing (or at least 10 times the respective elimination half-life, whichever is longer) except hormonal contraceptives and paracetamol; previous kidney transplantation; any history of allergy or sensitivity to migalastat or other iminosugars or excipients; any history of drug hypersensitivity, asthma, urticaria, or other severe allergic diathesis as well as current hay fever; any intercurrent illness or condition that may preclude the subject from fulfilling the protocol requirements or suggests to the investigator that the potential subject may have an unacceptable risk by participating in this study; any history of chronic or recurrent metabolic, renal, hepatic, pulmonary, gastrointestinal, neurological (particularly a history of epileptic seizures), endocrinological, immunological, psychiatric or cardiovascular disease, myopathies, and bleeding tendency; any active physical disease (acute or chronic); laboratory values outside the reference range that are of clinical relevance in the opinion of the investigator (e.g. suggesting an unknown disease and requiring further clinical evaluation assessed by the investigator) especially aspartate aminotransferase (AST), alanine aminotransferase (ALT), gamma glutamyl transpeptidase (gamma-GT); positive test for HIV antibodies or HIV-1 p24-antigen; positive HBsAg test; positive anti-HCV test; treatment with an investigational drug within 30 days of study start; inability to comply with study requirements or deemed otherwise unsuitable for study entry in the opinion of the investigator; female subject is pregnant or breastfeeding; blood donation within 30 days before signing informed consent to this study.

### Study Treatment

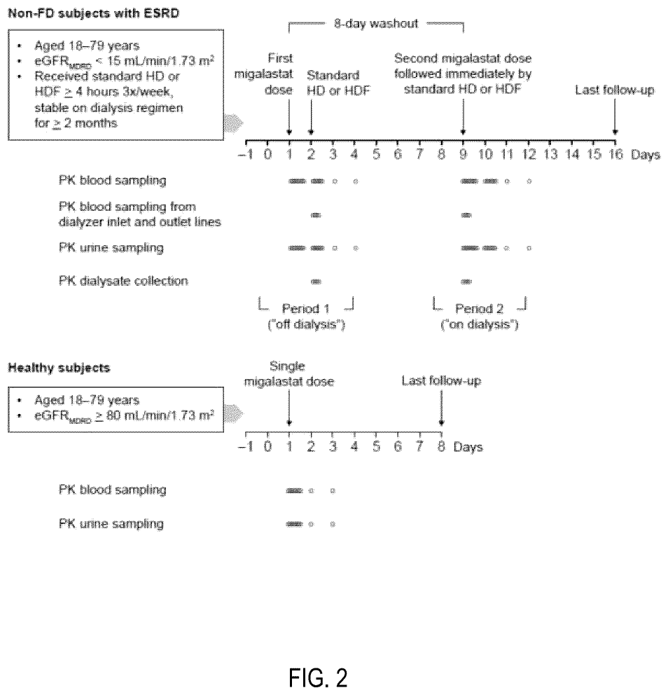

All subjects were screened before the first treatment period (day −21 to −2) for eligibility to participate in the trial. On day −1, all qualified subjects were admitted to the clinical testing facility to begin treatment and study assessments. Subjects with ESRD on standard HD or HDF participated in two treatment periods, receiving the following treatments in a fixed sequence, with a washout phase of at least 8 days between dosing, as illustrated in FIG. 2. Referring to FIG. 2, period 1 (“off dialysis”) consisted of a single oral dose of 150 mg (equivalent to 123 mg in free base form) migalastat hydrochloride (HCl) 24 hours before the start of dialysis (i.e., in a dialysis-free interval); period 2 (“on dialysis”) consisted of a single oral dose of 150 mg migalastat HCl immediately before the start of dialysis. Matched healthy control subjects with NRF participated in one treatment period and received a single oral dose of 150 mg migalastat.

### Pharmacokinetic (PK) Sample Collection

Subjects with ESRD on standard HD and HDF were domiciled at the clinical testing facility from the evening of day −1 (˜8 pm) until discharge the morning of day 3. In the ESRD group for Period 1 (dialysis initiated 24 hours post-dose), assessments included blood samples for plasma migalastat taken just prior to dosing (Time 0), and at 1, 2, 3, 4, 6, 8, 10, 12, 24, 48, and 72 hours post-dose, blood samples for plasma migalastat and dialysate samples for dialysate migalastat taken from the inlet line and outlet lines of the dialysis machine at 24:05, 25:00, 26:00, 27:00, 28:00, and 28:05 hh:mm post-dose. In the ESRD group for Period 2 (dialysis initiated immediately post-dose), assessments included blood samples for plasma migalastat taken just prior to dosing (Time 0), at 5 minutes post-dose concurrent with start of dialysis, and at 1, 2, 3, 4, 4:05, 6, 8, 10, 12, 24, 48, and 72 hours post-dose, blood samples for plasma migalastat and dialysate samples for dialysate migalastat taken from the inlet line and outlet lines of the dialysis machine at 00:05, 01:00, 02:00, 03:00, 04:00, and 04:05 hh:mm post-dose. Urine samples for urine migalastat collected on days 1 to 4 at intervals of 0 to 24 hours, 24 to 48 hours, and 48 to 72 hours, in both periods. In the NRF group, subjects were domiciled at the clinical testing facility from the evening of day −1 (˜8 pm) until discharge the morning of day 3. Assessments included blood samples for plasma migalastat taken just prior to dosing (Time 0), at 5 minutes post-dose, and at 1, 2, 3, 4, 6, 8, 10, 12, 24, and 48 hours post-dose, and urine samples for urine migalastat collected on days 1 and 2 at intervals of 0 to 24 hours and 24 to 48 hours. During the domiciled period, subjects with ESRD left the study center for dialysis. On day 4, these subjects returned to the study center for an ambulatory visit. Subjects in both groups had an ambulant follow-up visit 7 days after dosing.

### Analytical Method

Following completion of the in-clinic portion of the study, samples were shipped to the bioanalytical laboratory for measurement of migalastat concentrations. Migalastat was analyzed using a validated liquid chromatography-tandem mass spectrometry method. The analytical range of the plasma migalastat assay was 5.88 to 2940 ng/mL. The urine migalastat assay comprised two analytical ranges: a low range from 10 to 500 μg/mL and a high range from 100 to 5000 ng/mL. The dialysate migalastat analytical range was 0.1 to 40 μg/mL.

Individual plasma migalastat concentration-time profiles were plotted on a linear and log-linear scale using Prism GraphPad, version 7.01. Noncompartmental (NCA) PK analysis was performed on plasma, urine, and dialysate migalastat concentration-time data using Phoenix WinNonlin software, version 8.0.

Primary outcome measures for plasma migalastat PK parameters included: maximum plasma migalastat concentration (Cₘₐₓ), time of maximum plasma migalastat concentration (tₘₐₓ), plasma migalastat at the last measurable time point (Cₗₐₛₜ), plasma migalastat area under the curve (AUC) from Time 0 to the last measurable time point, t (AUC₀₋ₜ), plasma migalastat AUC from Time 0 extrapolated to infinity (AUC_{0-∞}), terminal elimination half-life (t_{1/2}), and apparent oral clearance (CL/F) for ESRD and healthy subjects.

Pre-dialyzer and post-dialyzer blood samples were also taken at limited selected timepoints from the inlet and outlet tubing of the dialysis machine for determination of the area under the concentration-time curve from venous samples entering the dialyzer (AUCᵢₙₗₑₜ) and the area under the concentration-time curve from arterial samples leaving the dialyzer (AUCₒᵤₜₗₑₜ), and mean plasma migalastat concentration during the dialysis interval (P). Pre- and post-dialyzer plasma migalastat AUCs were used to determine the dialysis extraction ratio (ED). Urine migalastat PK parameters were determined for ESRD subjects (if available) and healthy control subjects and included total amount of migalastat excreted in urine (Aₑ), the fraction of the migalastat dose recovered in urine (Fₑ), and renal clearance (CLR). Dialysate migalastat PK parameters were determined for ESRD subjects only and included dialysate clearance (CLD), migalastat concentration in dialysate (CD), dialyzer blood flow (QD), amount of migalastat recovered in dialysate (AₑD), and fraction of the migalastat dose recovered in dialysate (FₑD).

For descriptive statistics of plasma and dialysate migalastat concentrations, values below the lower limit of quantification (LLOQ) were set to missing. For calculation of the PK parameters, concentrations below LLOQ between two quantifiable concentrations, and trailing concentrations below LLOQ were set to missing. Otherwise, missing data were not replaced or imputed in any way.

Safety assessments for the PK study (secondary outcome measures) included treatment-emergent adverse events (TEAEs), clinical safety laboratory test results, and ECG results, and vital signs were measured.

### Results

### Subject Demographics

In total, 12 subjects were enrolled and completed the study between June and December 2019; six non-FD subjects with ESRD receiving dialysis (three on standard HD and three on HDF) and six controls with NRF matched for age, sex, and weight, as shown in Table 3. All 12 enrolled subjects were included in PK and safety analyses.

TABLE 3
Demographic characteristics of the enrolled subjects.

| Parameter | Statistic | ESRD^{a} (n = 6) | | NRF (n = 6) | |
|---|---|---|---|---|---|
| Age (years) | Mean | 47.5 | | 48.0 | |
| | Median | 45.0 | | 44.0 | |
| | (min-max) | (37-64) | | (33-69) | |
| Sex, n (%) | Female^{b} | 4 | (66.7) | 4 | (66.7) |
| | Male | 2 | (33.3) | 2 | (33.3) |
| Race, n (%) | White | 6 | (100) | 6 | (100) |

| Weight (kg) | Mean | 72.7 | 70.3 |
|---|---|---|---|
| | Median | 75.1 | 67.5 |
| | (min-max) | (56-83) | (60-82) |
| BMI (kg/m²) | Mean | 26.4 | 26.1 |
| | Median | 26.1 | 26.2 |
| | (min-max) | (20-31) | (21-34) |
| eGFR | Mean | 10.8 | 114.7 |
| (mL/min/1.73 m²) | Median | 8.0 | 98.5 |
| | (min-max) | (4-26)^{c} | (89-172)^{d} |

BMI = body mass index.
^{a}Pooled ESRD group combined subjects on HD and subjects on HDF.
^{b}Of the eight women included, five were of childbearing potential, two were at least 2 years postmenopausal, and one was surgically sterilized.
^{c}This included one subject with an initial eGFR of 25.8 mL/min/1.73 m²; the value was repeated due to low serum creatinine and the updated eGFR was calculated as 12.2 mL/min/1.73 m².
^{d}This included one subject with a low serum creatinine, making the eGFR artifactually high.

### Individual Plasma Migalastat Concentrations

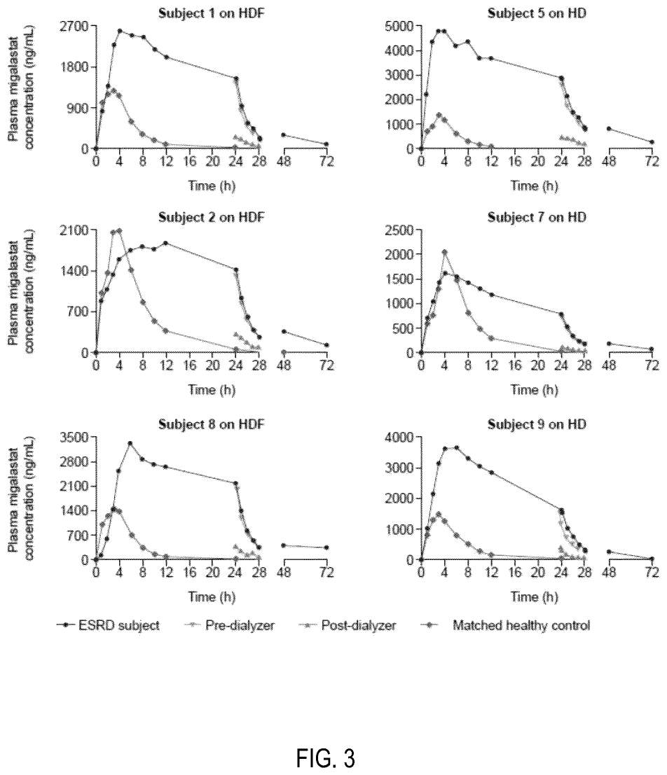

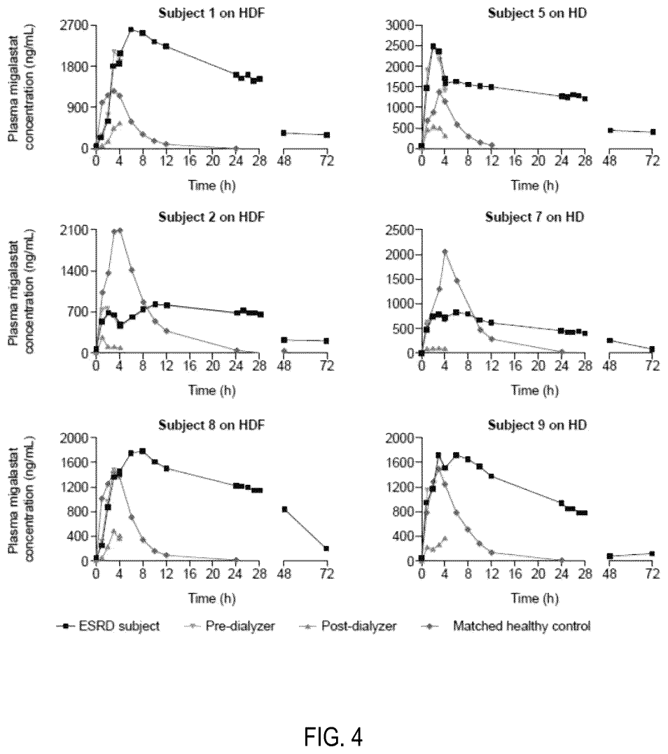

Individual plasma migalastat concentrations for ESRD subjects during periods 1 (“off dialysis”) and 2 (“on dialysis”) are shown in FIGS. 3 and 4, respectively, along with the concentration-time profiles of matched healthy controls.

All six subjects with ESRD had low but quantifiable migalastat concentrations above the LLOQ (5.88 ng/mL) at 72 hours in both periods. Quantifiable concentrations were negligible (<5% of Cₘₐₓ) pre-dose at period 2 (168 hours post-period 1 dosing) and did not impact period 2 PK assessments.

Generally, plasma migalastat concentration-time profiles appeared similar between subjects with ESRD receiving HD or HDF. Plasma concentrations peaked at approximately 5 to 6 hours post-dose in subjects with ESRD, and at approximately 3 to 4 hours post-dose in matched controls with NRF.

Plasma migalastat concentrations for each subject with ESRD during the “off-dialysis” period are shown in FIG. 3, including matched controls, for Period 1 (“off-dialysis period”), where the dose was administered at 24 hours before receiving dialysis treatment.

The concentrations shown in FIG. 3 were consistently higher than those during the Period 2 (“on-dialysis period”), where the dose was administered at 1 hour prior to dialysis. The results for Period 2 are shown in FIG. 4. Outside dialysis windows, plasma migalastat concentrations declined at a much slower rate in the subjects with ESRD than in matched controls with NRF. In all ESRD subjects, the concentration of plasma migalastat remained high until 24 to 28 hours post-dose (the 4-hour time interval of dialysis treatment) for subjects in the “off-dialysis” period, then declined precipitously to levels near the LLOQ. Post-dialyzer concentrations were substantially lower than the corresponding pre-dialyzer concentrations. Although both dosing-dialysis regimens removed a substantial amount of migalastat from plasma during the “on-dialysis” period, migalastat concentrations were appreciably greater than those of NRF subjects through 72 hours post-dose.

### Migalastat Pharmacokinetics in Plasma and Dialysate

Overall, plasma migalastat exposure was substantially higher in subjects with ESRD than in matched controls with NRF regardless of timing of dialysis, as shown in Table 4.

TABLE 4
Summary of Pharmacokinetic Parameters of Migalastat in Plasma.

| | Subjects with ESRD (n = 6) | | | | Subjects |
|---|---|---|---|---|---|
| PK | Period 1 (“off dialysis”) | | Period 2 (“on dialysis”) | | with NRF |
| parameter | ESRD-HD | ESRD-HDF | ESRD-HD | ESRD-HDF | (n = 6) |
| C_{max, 24 h} | | | | | |
| (ng/mL) | | | | | |
| Median | 3650 | 2600 | 1720 | 1770 | 1455 |
| Range | 1610-4770 | 1870-3320 | 816-2500 | 816-2620 | 1260-2070 |
| n | 3 | 3 | 3 | 3 | 6 |
| tₘₐₓ (h) | | | | | |
| Median | 4.0 | 6.0 | 3.0 | 8.0 | 3.0 |
| Range | 3.0-6.0 | 4.0-12.0 | 2.0-6.1 | 6.0-10.0 | 3.0-4.0 |
| n | 3 | 3 | 3 | 3 | 6 |
| AUC_{0-24 h} | | | | | |
| (ng*h/mL) | | | | | |
| Median | 61349 | 46160 | 31105 | 32515 | 9168 |
| Range | 26959-85764 | 37909-55216 | 14602-36337 | 16594-46401 | 6915-15926 |
| n | 3 | 3 | 3 | 3 | 6 |
| AUC₀₋ₜ | | | | | |
| (ng*h/mL) | | | | | |
| Median | 74169 | 59072 | 45147 | 68668 | 9168 |
| Range | 35210-121553 | 52914-76048 | 26672-67965 | 33308-79321 | 6698-16568 |
| n | 3 | 3 | 3 | 3 | 6 |
| AUC_{0-∞} | | | | | |
| (ng*h/mL) | | | | | |
| Median | 73068 | 115051 | 47544 | 74400 | 9237 |
| Range | 48688-97448 | — | 29118-84727 | 40661-87626 | 6919-16628 |
| n | 2 | 1 | 3 | 3 | 6 |
| t_{1/2} (h) | | | | | |
| Median | 17.3 | 31.1 | 20.9 | 19.6 | 3.5 |
| Range | 15.4-19.2 | — | 14.7-29.2 | 18.7-25.1 | 2.0-5.9 |
| n | 2 | 1 | 3 | 3 | 6 |
| CL/F (L/h) | | | | | |
| Median | 2.31 | 1.30 | 3.15 | 2.02 | 16.3 |
| Range | 1.54-3.08 | — | 1.77-5.15 | 1.71-3.69 | 9.02-21.7 |
| n | 2 | 1 | 3 | 3 | 6 |
| V_{z}/F (L) | | | | | |
| Median | 59.7 | 58.4 | 74.6 | 56.9 | 73.5 |
| Range | 34.1-85.3 | — | 66.9-155.4 | 46.2-133.6 | 59.4-97.1 |
| n | 2 | 1 | 3 | 3 | 6 |

AUC_{0-∞}, area under the concentration-time curve from time 0 to infinity; AUC_{0-24 h}, area under the concentration-time curve from time 0 to 24 hours; AUC₀₋ₜ, area under the concentration-time curve from time 0 to the last measurable concentration; CL/F, apparent plasma clearance; C_{max, 24 h}, maximum concentration observed between time 0 to 24 hours; t_{1/2}, apparent terminal elimination half-life; tₘₐₓ, time to maximum concentration; V_{z}/F, apparent terminal phase volume of distribution.
^{a} AUC_{0-∞} and other extrapolated parameters are not reported for three ESRD-HDF subjects because >50% of area was extrapolated for these subjects.

The difference was more pronounced when comparing subjects with ESRD to matched controls with NRF when administration was during period 1 (“off dialysis”), with median area under the concentration-time curve from time 0 to 24 hours (AUC_{0-24 h}) approximately 5.9-fold but median Cₘₐₓ only 2.1-fold higher than in matched controls with NRF, respectively. During period 2 (“on dialysis”), the difference was somewhat lower, with median AUC_{0-24 h} approximately 3.5-fold higher and median Cₘₐₓ only 1.2-fold higher than in matched controls with NRF. The median rate of absorption was prolonged by 1 to 5 hours in ESRD subjects relative to subjects with NRF. Median terminal half-life increased by 5- to 9-fold and, correspondingly, median apparent clearance decreased by approximately 5- to 12-fold in ESRD subjects relative to subjects with NRF.

A summary of migalastat PK parameters in dialysate and extraction from plasma is shown in Table 5. The median fraction of the dose recovered in dialysate was similar between the Period 1 “off dialysis” (22.2%) and Period 2 “on dialysis” (22.1%) treatment periods. Comparison of median fraction of the dose recovered in dialysate between dialysis methods, standard HD versus HDF, were generally similar also (standard HD, 21.2%; HDF, 22.6%). Overall, median apparent dialysate clearance (CLD) was similar between treatment periods: 8.81 L/h for “off dialysis” and 11.1 L/h for “on dialysis”. Median CLD was generally similar between dialysis methods (standard HD, 6.13 L/h; HDF, 10.2 L/h). Both methods of dialysis demonstrated significant decreases in plasma migalastat exposures during 4-hour dialysis treatments as measured by the ratio between AUCᵢₙₗₑₜ and AUCₒᵤₜₗₑₜ. The ratio is expressed as percent of the absorbed dose extracted by dialysis. Overall, median percent of the absorbed dose extracted by dialysis was similar between treatment periods: 73.3% extracted during “off dialysis” and 76.3% during “on dialysis”. Median percent of the absorbed dose extracted by dialysis comparisons between dialysis methods were similar (standard HD, 74.1%; HDF, 71.8%).

TABLE 5
Summary of PK Parameters of Migalastat in Dialysate
and Extraction from Plasma by Dialysis.

| | Subjects with ESRD (n = 6) | | | |
|---|---|---|---|---|
| PK | Period 1 (“off dialysis”) | | Period 2 (“on dialysis”) | |
| parameter | ESRD-HD | ESRD-HDF | ESRD-HD | ESRD-HDF |
| FₑD (%) | | | | |
| Median | 21.8 | 22.6 | 32.8 | 17.1 |
| Range | 6.37-25.3 | 12.5-27.9 | 17.9-45.4 | 15.7-26.3 |
| n | 3 | 3 | 3 | 3 |
| CL_{D} (L/h) | | | | |
| Median | 6.13 | 10.2 | 10.6 | 11.9 |
| Range | 6.01-10.8 | 7.39-12.2 | 9.44-11.5 | 6.60-12.3 |
| n | 3 | 3 | 3 | 3 |
| AUCᵢₙₗₑₜ | | | | |
| (ng*h/mL) | | | | |
| Median | 2859 | 2465 | 4756 | 3456 |
| Range | 1342-5255 | 2151-3422 | 2453-7354 | 2366-4062 |
| n | 3 | 3 | 2 | 3 |
| AUCₒᵤₜₗₑₜ | | | | |
| (ng*h/mL) | | | | |
| Median | 784 | 732 | 924 | 993 |
| Range | 251-1363 | 607-890 | 376-1694 | 576-1005 |
| n | 3 | 3 | 3 | 3 |
| ED (%) | | | | |
| Median | 74.1 | 71.8 | 80.6 | 75.6 |
| Range | 72.6-81.3 | 70.3-74.0 | 77.0-84.7 | 70.9-75.6 |
| n | 3 | 3 | 3 | 3 |

AUCᵢₙₗₑₜ, area under the concentration-time curve from venous samples entering the dialyzer; AUCₒᵤₜₗₑₜ, area under the concentration-time curve from arterial samples leaving the dialyzer; CL_{D}, apparent dialysate clearance; ED, dialysis extraction ratio expressed as [(AUCiₙₗₑₜ − AUCₒᵤₜₗₑₜ)/AUCᵢₙₗₑₜ] * 100; ESRD, end-stage renal disease; FₑD, fraction of the migalastat dose recovered in dialysate.

A summary of migalastat PK parameters in urine is shown in Table 6.

TABLE 6
Summary of PK parameters of migalastat in urine.

| | Subjects with ESRD (n = 6) | | | | Subjects |
|---|---|---|---|---|---|
| PK | Period 1 (“off dialysis”) | | Period 2 (“on dialysis”) | | with NRF |
| parameter | ESRD-HD | ESRD-HDF | ESRD-HD | ESRD-HDF | (n = 6) |
| Aₑ (mg) | | | | | |
| Median | 9.4 | 3.8 | 6.0 | 0.8 | 59.4 |
| Range | 2.1-20.3 | 1.3-5.4 | 0.1-8.9 | 0.7-2.9 | 35.8-83.0 |
| n | 3 | 3 | 3 | 3 | 6 |
| Fₑ (%) | | | | | |
| Median | 6.3 | 2.6 | 4.0 | 0.6 | 39.6 |
| Range | 1.4-13.5 | 0.9-3.6 | 0.1-5.9 | 0.4-1.9 | 23.9-55.3 |
| n | 3 | 3 | 3 | 3 | 6 |
| CL_{R} (L/h) | | | | | |
| Median | 0.266 | 0.065 | 0.198 | 0.025 | 5.492 |
| Range | 0.017-0.273 | 0.025-0.070 | 0.002-0.226 | 0.008-0.042 | 4.928-7.022 |
| n | 3 | 3 | 3 | 3 | 6 |

Median fraction of the migalastat dose excreted in urine (Fₑ%) and corresponding amount recovered in urine (Aₑ) in healthy subjects with NRF was approximately 39.6% (Aₑ, 59.4 mg) compared with 3.1% (Aₑ, 4.59 mg) excreted during Period 1 “off dialysis” and 1.2% (Aₑ, 1.85 mg) excreted during Period 2 “on dialysis” in subjects with ESRD. Median renal clearance (CLᵣ) was considerably lower for subjects with ESRD than healthy subjects with NRF (0.068 and 0.034 L/h for “off dialysis” and “on dialysis”, respectively, in subjects with ESRD vs. 5.49 L/h for healthy subjects with NRF).

### Example 2. Population Pharmacokinetic (PopPK) Analysis

A popPK analysis (model optimization) was performed using nonlinear mixed-effects modeling (NONMEM®) program version 7.4.4 (ICON Development Solutions, Ellicott City, MD, USA).

An earlier popPK model based on data from the original Phase III study in patients with FD served as the starting model (referred to in original publication as the Interim 3 Adult model); this model is described in detail in Leonowens C et al. 2022. This base model has a two-compartment structure with an absorption lag time and an absorption rate (Ka) that is related to time after dose up to 24 hours using a slope-intercept relationship. The effects of body weight (up to 70 kg) on PK parameters were described using allometric scaling, with an allometric exponent of 0.75 on CL/F and apparent intercompartmental clearance (Q/F), and allometric exponent of 1 on apparent volume of distribution of the central (V₂/F) and peripheral (V₃/F) compartments. Above 70 kg, there did not appear to be any correlation between body weight and CL/F, Q/F, V₂/F and V₃/F. Additionally, renal function affects CL/F and FD status affects both CL/F and V₂/F. The effect of renal function on CL/F is characterized as a piecewise function in which CL/F is linearly dependent on estimated glomerular filtration rate (eGFR) until 120 mL/min/1.73 m², above which the effect is a constant. FD status is characterized as a binary effect. Interindividual variability (IIV) is included on CL/F and V₂/F with correlation and on the slope and intercept on Ka.

Several possible dialysis-related changes to the model were considered: increase in apparent plasma clearance (CL/F) due to apparent dialysis clearance (CL_{dialysis}) during active dialysis; volume changes due to dialysis and fluid accumulation between dialysis; and separate residual variability during dialysis. Model selection considered change in objection function value, goodness-of-fit plots, magnitude of interindividual and residual variabilities, precision of estimates, successful minimization, and physiologic rationale.

The selected model was used for Monte Carlo simulations of the exposures of FD patients with ESRD after various dosing regimens.

For the simulations, two sets of 100 virtual FD subjects were simulated. The set representing subjects with NRF simulated weight and estimated glomerular filtration rate (eGFR) as normal distribution based on the weight (72.5±15.4 kg) and eGFR (87.8±31.7 mL/min/1.73 m²) of FD patients in Study AT1001-011. For the set representing subjects with ESRD, the eGFR was changed to be simulated as a uniform distribution between 5 and 15 mL/min/1.73 m². Dialysis clearance differs within and between subjects with no clear predictable pattern; therefore, dialysis clearance in the ESRD population was simulated as a uniform distribution assuming the range from the current study. Simulation scenarios evaluated twice-weekly and thrice-weekly dialysis and a migalastat HCl dose of 100 or 150 mg administered weekly (QW) or every other week (QOW).

Simulated concentration-time data were used to determine Cₘₐₓ, concentration at the end of a dosing interval at steady state (C_{trough}), and the average plasma concentration (C_{avg}). C_{avg} was calculated as the AUC for a steady-state dosing interval (AUCₜₐᵤ) divided by the duration of the dosing interval. The PK parameters after each of these two dosing scenarios were compared with the standard migalastat dosing (150 mg every other day [QOD]) in subjects with NRF by calculating the geometric mean ratio (ESRD: NRF) and the 90% confidence interval. Migalastat exposures in each ESRD group and NRF subjects were considered bioequivalent if the 90% CIs of the geometric mean ratio of Cₘₐₓ or AUCₜₐᵤ were within the range of 0.8 to 1.25 (i.e., 80% to 125%).

### Results

The popPK model was modified to incorporate dialysis clearance on apparent clearance during each dialysis session. Dialysis-related changes in volume or residual error were not incorporated because these changes did not improve the goodness-of-fit plots or residual variability. The parameter estimates of the final models are included in Table 7:

TABLE 7
PopPK model structure and parameter estimates.

| | | | Estimate (relative | |
|---|---|---|---|---|
| | | | standard error) | |
| | | | [shrinkage] | |
| | | | Fixed | IIV or |
| Parameter | Equation | Label | effect | residual |
| Ka (h⁻¹) | Ka = | 1. Intercept on Ka | 0.256 | 60.4% |
| | $max \{ \begin{matrix}0.00001 \\ {θ}_{1} \times exp ( {η}_{1} ) + ( {θ}_{2} \times exp ( {η}_{2} ) \times TADC\end{matrix}$ | | (9%) | (11%) [30%] |
| | | 2. Slope for time- | 0.284 | 60.7% |
| | | dependent effect | (9%) | (9%) |
| | | on Ka (with a | | [31%] |
| | | maximum time of | | |
| | | 24 h) | | |
| ALAG1 | ALAG1 = θ₃ | 3. Lag time | 0.175 | — |
| (h) | | | (5%) | |
| F1 | F1 = θ₄ | 4. Bioavailability | 1 FIX | — |
| CL/F (L/h) | $\frac{CL}{F} = if {( eGFR > 1 \; 20 , {θ}_{5} , {θ}_{6} \times \frac{eGFR}{90} )}^{{θ}_{7}} \times$ $( 1 + {θ}_{8} \times ( 1 - FABRY ) ) \times {WTCO}^{{θ}_{9}} \times exp ( {η}_{5} )$ | 5. Coefficient for eGFR effect on CL/F for FD subjects with | 20.9 (17%) | 28.8% (7%) [4%] |
| | | eGFR > | | |
| | | 120 mL/min/1.73 | | |
| | | m² and weight ≥ | | |
| | | 70 kg | | |
| | | 6. Coefficient for | 18.6 | — |
| | | eGFR effect on | (16%) | |
| | | CL/F for FD | | |
| | | subjects with | | |
| | | eGFR = 90 | | |
| | | mL/min/1.73 m² | | |
| | | and weight ≥ 70 | | |
| | | kg | | |
| | | 7. Exponent for | 0.922 | — |
| | | eGFR effect on | (6%) | |
| | | CL/F | | |
| | | 8. Fractional | -0.15 | — |
| | | change in CL/F in | (25%) | |
| | | subjects without | | |
| | | FD | | |
| | | 9. Exponent for | 0.75 | — |
| | | weight effect on | FIX | |
| Q/F (L/h) | $\frac{Q}{F} = {θ}_{10} \times {WTCO}^{{θ}_{9}}$ | CL/F and Q/F | | |
| | | 10. Q/F for | 1 | — |
| | | subjects with | (5%) | |
| | | weight ≥ 70 kg | | |
| V₂/F (L) | $\frac{{V}_{2}}{F} = {θ}_{11} \times ( 1 + {θ}_{12} \times ( 1 - FABRY ) ) \times$ ${WTCO}^{{θ}_{13}} \times exp ( {η}_{11} )$ | 11. Typical value for V₂/F for FD subjects with weight ≥ 70 kg | 70.1 (5%) | 34.5% (6%) [7%] |
| | | 12. Fractional | −0.30 | — |
| | | change in V₂/F in | 6 | |
| | | subjects without | (13%) | |
| | | FD | | |
| | | 13. Exponent for | 1 FIX | — |
| V₃/F (L) | $\frac{{V}_{3}}{F} = {θ}_{14} \times {WTCO}^{{θ}_{13}}$ | weight effect on V₂/F and V₃/F | | |
| | | 14. V₃/F for FD | 27.5 | — |
| | | subjects with | (12%) | |
| | | weight ≥ 70 kg | | |
| Residual | Combined proportional and additive | Proportional (%) | — | 26.2% |
| error | residual error | | | (5.5%) |
| | | | | [7%] |
| | | Additive (ng/ml) | — | 2.55 |
| | | | | (30%) |
| | | | | [7%] |

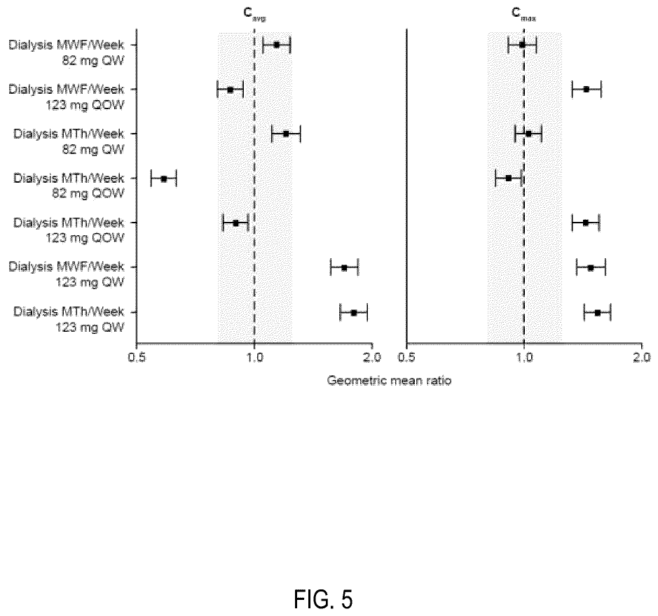

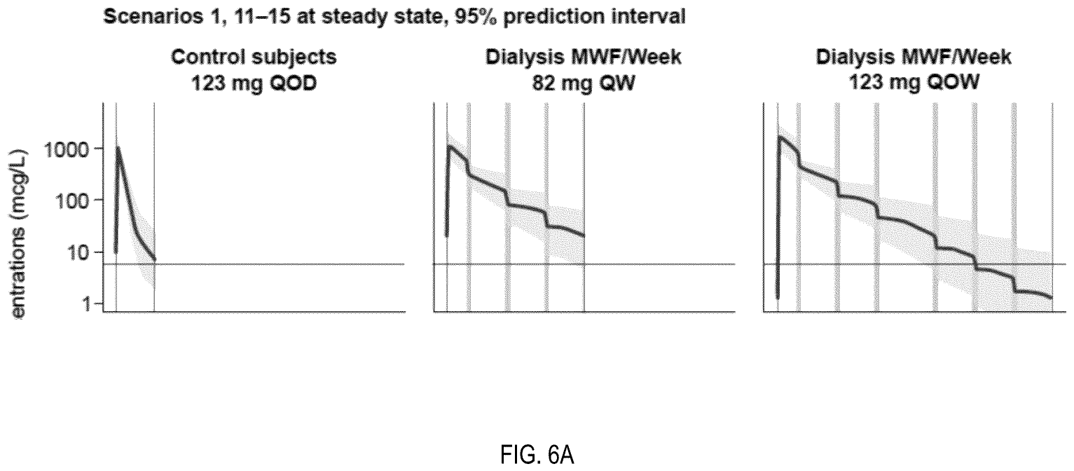

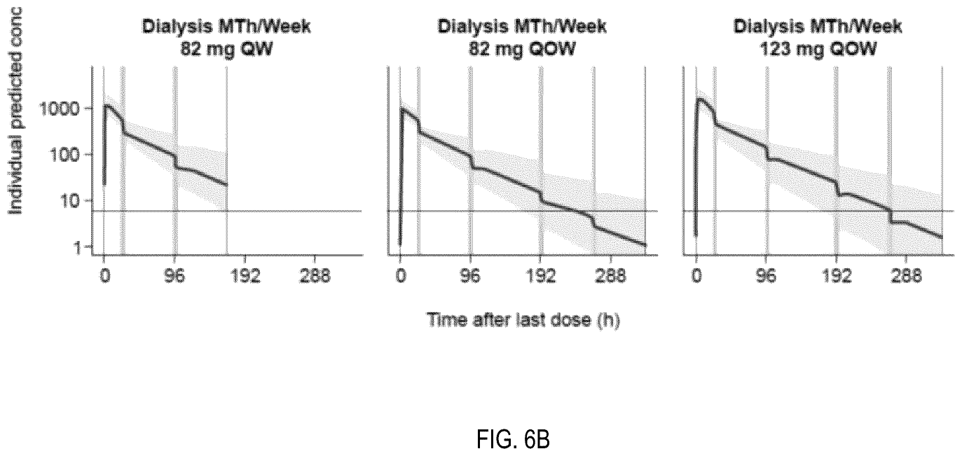

PopPK simulations showed that migalastat 82 mg QW dosing along with twice- or thrice-weekly dialysis was predicted to result in migalastat C_{avg} and Cₘₐₓ values that are approximately bioequivalent to those of subjects with NRF receiving 123 mg of migalastat QOD (FIGS. 5 and 6). Few subjects attained Cₘₐₓ>10 μM or achieved concentrations that were below the limit of quantification (BLQ) at the end of a dosing interval at steady state (Table 4), which are hypothesized to facilitate intracellular trafficking of mutant α-Gal A to the lysosome and dissociation of the migalastat and α-Gal A complex.

PopPK simulations also showed that Migalastat 123 mg QOW along with twice- or thrice-weekly dialysis was predicted to result in a C_{avg} that was bioequivalent to, but with a higher Cₘₐₓ than, subjects with NRF receiving 123 mg of migalastat QOD, as shown in FIG. 5 and FIG. 6.

FIG. 5 shows geometric mean ratios for C_{avg} and Cₘₐₓ for week-based dosing and dialysis schedule scenarios. Referring to FIG. 5, MTh is Monday, Thursday; MWF is Monday, Wednesday, Friday; and Q is “every”. The black dashed line is the geometric mean ratio of 1.0; gray shaded region is the bioequivalence criteria for 0.8 to 1.25 for the 90% confidence interval of the geometric mean ratio; dot and segment are the geometric mean ratio and 90% confidence interval.

FIG. 6 shows median (95% prediction interval) steady-state concentration-time profiles for week-based dosing and dialysis schedule scenarios. The black line is the median with 97.5th prediction interval; horizontal line is the LLOQ; vertical line is the migalastat dose; shaded gray vertical bars denote the dialysis periods.

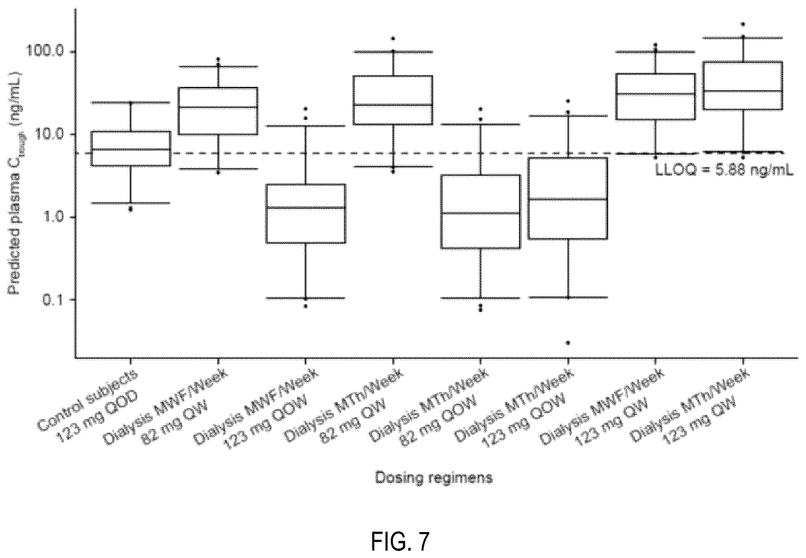

Migalastat 123 mg QOW regimens resulted in a substantial proportion of the subjects who attained Cₘₐₓ>10 UM and/or C_{trough} that was below quantification level (BQL; Table 4 and FIG. 7).

PopPK simulations also showed that migalastat 82 mg QOW resulted in a Cₘₐₓ and C_{avg} that was too low, while migalastat 123 mg QW resulted in a Cₘₐₓ and C_{avg} that were high, relative to subjects with NRF.

Table 8 shows the results across all simulation scenarios:

TABLE 8
Results across all simulation scenarios

| | | | | | Subjects (%) | |
|---|---|---|---|---|---|---|
| | Dialysis | Migalastat | Bioequivalent? | | Cₘₐₓ >10 | C_{trough} |
| Scenario | frequency | regimen | C_{avg} | Cₘₐₓ | μM | BLQ |

Prespecified scenarios

| | | | | | | |
|---|---|---|---|---|---|---|
| 1 | N/A | 123 mg | Reference | Reference | 17 | 44 |
| | (control) | QOD | | | | |
| 2 | Q3D | 123 mg | High | High | 59-77 | 0 |
| | | QOD | | | | |
| 3 | QOD | 123 mg | High | High | 82 | 0 |
| | | QOD | | | | |
| 4 | QOD | 123 mg | High | High | 65 | 0 |
| | | Q4D | | | | |
| 5 | Q3D | 123 mg | High | High | 71 | 0 |
| | | Q3D | | | | |

Extended dosing interval scenarios

| | | | | | | |
|---|---|---|---|---|---|---|
| 6 | QOD | 49 mg | Slightly | Low | 1 | 0 |
| | | Q4D | high | | | |
| 7 | QOD | 65 mg | Yes | Slightly | 4 | 4 |
| | | Q6D | | low | | |
| 8 | QOD | 123 mg | Yes | High | 56 | 79 |
| | | Q12D | | | | |
| 9 | Q3D | 82 mg | High | Yes | 14 | 0 |
| | | Q6D | | | | |
| 10 | Q3D | 123 mg | Yes | High | 59 | 60 |
| | | Q12D | | | | |

Week-based dosing and dialysis schedule

| | | | | | | |
|---|---|---|---|---|---|---|
| 11 | MWF | 82 mg | Yes | Yes | 15 | 8 |
| | QW | QW | | | | |
| 12 | MWF | 123 mg | Yes | High | 56 | 92 |
| | QW | QOW | | | | |
| 13 | MTh | 82 mg | Slightly | Yes | 12 | 7 |
| | QW | QW | high | | | |
| 14 | MTh | 82 mg | Low | Yes | 8 | 90 |
| | QW | QOW | | | | |
| 15 | MTh | 123 mg | Yes | High | 49 | 78 |
| | QW | QOW | | | | |
| 20^{a} | MWF | 123 mg | High | High | 53 | 2 |
| | QW | QW | | | | |
| 21^{a} | MTh | 123 mg | High | High | 58 | 1 |
| | QW | QW | | | | |

Twice QOW dosing and week-based dialysis schedule

| | | | | | | |
|---|---|---|---|---|---|---|
| 16 | MTh | 82 mg | Yes | Yes | 12 | 56 |
| | QW | SuW | | | | |
| | | QOW | | | | |
| 17 | MWF | 82 mg | Yes | Yes | 15 | 62 |
| | QW | SuW | | | | |
| | | QOW | | | | |
| 18 | MTh | 82 mg | Slightly | Yes | 17 | 54 |
| | QW | SuTu | high | | | |
| | | QOW | | | | |
| 19 | MWF | 82 mg | Yes | Yes | 11 | 75 |
| | QW | SuTu | | | | |
| | | QOW | | | | |

The simulations (along with all the simulation scenarios, which are detailed in Table 8) suggested that migalastat 82 mg QW or migalastat 123 mg QOW could be acceptable doses in ESRD, but an understanding of tissue concentrations (hence, need for PBPK model described below) was needed to evaluate whether there is an advantage of migalastat 123 mg QOW when compared with migalastat 82 mg QW.

Table 9 shows simulation scenarios for QW and QOW dosing and week-based dialysis, where bioequivalence was determined by comparing with predictions in virtual subjects with NRF.

TABLE 9
Simulation Scenarios for QW and QOW Dosing and Week-based Dialysis.

| | Dialysis | Migalastat | | Bioequivalent? | | Subjects (%) | |
|---|---|---|---|---|---|---|---|
| Scenario | Frequency | Regimen | | C_{avg} | Cₘₐₓ | Cₘₐₓ | C_{trough} |
| Reference | None | 123 | mg QOD | Reference | Scenario | 17 | 44 |
| Test 1 | MWF QW | 82 | mg QW | Yes | Yes | 15 | 8 |
| Test 2 | MWF QW | 123 | mg QOW | Yes | High | 56 | 92 |
| Test 3 | MTh QW | 82 | mg QW | Slightly high | Yes | 12 | 7 |
| Test 4 | MTh QS | 82 | mg QOW | Low | Yes | 8 | 90 |
| Test 5 | MTh QW | 123 | mg QOW | Yes | High | 49 | 78 |
| Test 6^{a} | MWF QW | 123 | mg QW | High | High | 53 | 2 |
| Test 7^{a} | MTh QW | 123 | mg QW | High | High | 58 | 1 |

BLQ, below the limit of quantification; C_{avg}, average concentration; Cₘₐₓ, maximum concentration; C_{trough}, concentration at the end of a dosing interval at steady state; MWF, Monday, Wednesday, Friday; MTh, Monday, Thursday; NRF, normal renal function; QOW, every other week; QOD, every other day; QW, every week.
^{a}These simulations were added after the twice every other week dosing simulations were conducted.

FIG. 7 shows the predicted C_{trough} for week-based dosing and dialysis schedule scenarios. Referring to FIG. 7: C_{trough}, concentration at the end of a dosing interval at steady state; LLOQ, lower level of quantification; MTh, Monday, Thursday; MWF, Monday, Wednesday, Friday; QW, every week; QOW, every other week, QOD, every other day. The box plot represents the median, 25th, and 75th percentile predicted concentrations. The whisker ends represent the 2.5th and 97.5th percentile values. The dashed line is the LLOQ for migalastat detection (5.88 ng/ml).

### Example 3: Physiologically Based Pharmacokinetic (PBPK) Modeling

PBPK modeling was conducted in PK-Sim Version 8 Build 22 (Bayer, Leverkusen, Germany). Data obtained during the PK study in non-FD subjects with ESRD receiving dialysis treatment and matched healthy subjects were combined with subjects from the Phase III study, AT1001-011, conducted in patients with FD and an eGFR ≥60 mL/min/1.73 m2. These combined data were used to update the previous PBPK model to add components to describe dialysis to allow appropriate migalastat dose regimens for evaluation in a future clinical trial in FD patients with ESRD on dialysis treatment. The previous PBPK model was described in WU Y S, Khanna R, Schmith V, Lun Y, Shen J S, Garcia A, et al., Migalastat Tissue Distribution: Extrapolation from Mice to Humans Using Pharmacokinetic Modeling and Comparison with Agalsidase Beta Tissue Distribution in Mice. Clin. Pharmacol. Drug. Dev. 2021, 10(9):1075-88, which is incorporated herein by reference in its entirety.

In this previous study, two PBPK models (full dataset and steady state) were developed that featured migalastat clearance from renal filtration, small hepatic metabolism, and minor extrahepatic glucuronidation. The model was modified to evaluate overall metabolic elimination as an enzymatic process to allow for modeling of decreased clearance in ESRD subjects. Inputs defining the two models are summarized in Table 10:

TABLE 10
Parameters for the migalastat PBPK models in humans

| | | |
|---|---|---|
| | | Steady- |
| | Full | state |
| | dataset | dataset |
| Parameter | model | model |

Basic physiochemical properties

| Is it a small molecule? | Yes | |
|---|---|---|
| Lipophilicity | −1.70 | −3.03 |
| Fraction unbound (%) | 100 | |
| Molecular weight (g/mol) | 163.17 | |
| Has halogens? | No | |
| Compound type | Monoprotic base | |
| pKa (basic) | 7.47 | |
| Solubility | 500 g/L between pH 1.2 and 7.5 | |
| Partition coefficient calculation method | Schmitt | |
| Cellular permeabilities calculation | PK-Sim ® standard | |
| method | | |

Biological properties

| | | |
|---|---|---|
| Specific intestinal permeability | 1 × 10⁻⁴ | 2 × 10⁻⁵ |
| (cm/min) | | |
| Specific binding - lysosome: K_{d} (M) | 0.01 | 0.01 |
| Specific binding - lysosome: K_{off} | 1.6 × 10⁻³ | 2 × 10⁻³ |
| (min⁻¹) | | |
| Hepatic clearance - specific clearance | 0.05 | 0.04 |
| (min⁻¹) | | |
| Extrahepatic clearance - UDPGT in | 2.10 × 10⁻³ | 2.10 × 10⁻³ |
| brain CLspec/[enzyme] (L/μmol/min) | | |

Tissue partition coefficient (intracellular: plasma)

| | | |
|---|---|---|
| Heart | 0.01 | 5 × 10⁻³ |
| Kidney | 4.00 | 0.76 |
| Liver | 0.10 | 5 × 10⁻³ |
| Muscle | 0.50 | 0.79 |
| Skin | 0.54 | 0.54 |
| Brain | 0.81 | 0.80 |

Virtual ESRD subjects were created from subjects with NRF by fitting glomerular filtration rate (GFR) and theoretical hepatic enzyme expression level to the observed plasma concentration-time data of ESRD subjects in the current study. Dialysis was modeled by addition of a dialysis compartment, implementation of dialysis-dependent passive transport processes, and creation of dialysis events in the simulation. The PBPK models were used to simulate 100 and 150 mg migalastat HCl administered QW or QOW and twice- or thrice-weekly dialysis treatment. In lymphoblasts and fibroblasts isolated from males with FD, 75 different missense mutant forms of the α-Gal A enzyme, one insertion, and one splice-site mutation were identified. Of these, 49 missense mutant forms had half maximal effective concentration (EC₅₀) values ranging from 820 nM to >1 mM. An approximate median EC₅₀ of 1 μM was selected as a target for simulations of time above EC₅₀ in ESRD subjects with FD. The predicted total (free+bound) tissue concentrations were evaluated for the time above the concentration producing 50% of maximum effect (EC₅₀; 1 μM) during a dosing interval (F_{EC50}) or time above EC₅₀ over 2 weeks.

The full dataset PBPK model and the steady-state PBPK model from the original PBPK model (Wu Y S, et al.) were modified for use in subjects both with and without renal impairment. The extra-renal clearance (a minor elimination pathway) was changed from an overall hepatic clearance process to an enzyme metabolism process in the liver, such that individual clearance can be varied by controlling the expression level of the hepatic enzyme in the virtual subject. Based on the popPK analysis, apparent clearance is proportional to renal clearance; therefore, a virtual subject with ESRD was created by decreasing the GFR to the median eGFR of the dialysis subjects in the study and decreasing the hepatic enzyme expression proportionally, fitting to the plasma concentration-time profiles of ESRD subjects before dialysis in the off-dialysis period. The dialysis process was modeled by adding a dialysis compartment in the PBPK model that filters migalastat for 4 hours when a dialysis event is triggered.

The PBPK models were used to predict migalastat tissue concentrations for the top two dosing regimens selected using popPK analysis. The predicted fractions of dosing interval with tissue concentration above EC₅₀ are presented in Table 11. Based on either model, the 123 mg QOW regimen in ESRD subjects produced a more similar fraction of time above EC₅₀ to the reference case of subjects without renal impairment receiving 123 mg QOD, while migalastat 82 mg administered QW in ESRD subjects was associated with greater fraction of time above EC₅₀, as shown in Table 11. However, the 82 mg QW dose resulted in a longer duration of concentrations above the LLOQ and was consequently more vulnerable to accumulation. The migalastat 123 mg QOW dose predicted an adequate Cₘₐₓ with accompanying C_{trough} levels at or near the LLOQ (similar to NRF) after a minimum of four dialysis sessions.

TABLE 11
Predicted Fraction of Dosing Interval Above EC₅₀ in the
Tissues for Typical Male Subjects with Weight of 73 kg.

| | | | | | | | |
|---|---|---|---|---|---|---|---|
| | | | | | | | Small |
| | | Brain | Heart | Kidney | Liver | Skin | intestine |
| Dosing | Dialysis | (%) | (%) | (%) | (%) | (%) | (%) |

Week-based dosing and dialysis schedule

| | | | | | | | |
|---|---|---|---|---|---|---|---|
| 123 mg QOD in | None | 0 | 5.00 | 40.1 | 14.5 | 19.9 | 25.2 |
| healthy subject | | | | | | | |
| 123 mg QOW in | 2x/week | 0 | 6.77 | 29.0 | 12.9 | 17.3 | 21.9 |
| subject with ESRD | 3x/week | 0 | 6.77 | 27.2 | 12.9 | 17.3 | 21.3 |
| 82 mg QW in | 2x/week | 0 | 12.8 | 56.8 | 19.2 | 27.4 | 35.4 |
| subject with ESRD | 3x/week | 0 | 12.8 | 47.9 | 19.2 | 27.4 | 35.4 |

Steady-state dataset model

| | | | | | | | |
|---|---|---|---|---|---|---|---|
| 123 mg QOD in | None | 0 | 7.71 | 35.0 | 11.0 | — ^{a} | 22.5 |
| healthy subject | | | | | | | |
| 123 mg QOW in | 2x/week | 0 | 7.59 | 41.5 | 11.5 | — ^{a} | 24.6 |
| subject with ESRD | 3x/week | 0 | 7.59 | 35.9 | 11.5 | — ^{a} | 21.4 |
| 82 mg QW in | 2x/week | 0 | 13.8 | 67.0 | 15.8 | — ^{a} | 38.5 |
| subject with ESRD | 3x/week | 0 | 13.8 | 71.1 | 15.8 | — ^{a} | 38.5 |

EC50, concentration producing 50% of maximum effect;
^{a} Skin tissue exposure was only observed in the single-dose mouse biodistribution study, so steady-state dataset model was not built to predict skin exposure.

### Results and Discussion

Th PBPK study, together with the popPK study and the Phase 1 clinical study, investigated the PK, safety, and dialyzability of migalastat in non-FD subjects with ESRD undergoing standard HD and HDF. A popPK model and a PBPK model developed using the clinical data allowed simulations to be conducted to determine the appropriate dose of migalastat in patients with ESRD that would result in exposures, in the plasma and the tissues, that would be appropriate, relative to the dose of migalastat in patients with FD who do not have ESRD.

Currently, migalastat is not recommended in patients with an eGFR <30 mL/min/1.73 m². However, as expected from its physical and chemical characteristics of being a highly soluble small molecule, results showed that migalastat was dialyzable with an extraction ratio of approximately 74 to 81% of the fraction of the dose in circulation after 24 hours post-dose. Additionally, deteriorated renal function resulted in only approximately <5% of the dose recovered in urine over a 1-week period and about 20% recovered in dialysate, compared with 77% of the dose excreted in the urine from subjects with NRF. Although one dialysis treatment contributed substantially to migalastat elimination, a significant fraction of the dose remains in plasma. While overall AUC increased considerably with decreasing renal function, the Cₘₐₓ increased to a smaller degree, likely because this PK parameter is related to absorption and volume of distribution and not clearance. The observed increased exposure in subjects with ESRD compared with the matched controls with NRF in this study is in line with results observed in a previous study (AT1001-015), where it was shown that migalastat plasma clearance decreased significantly in severe renal impairment, leading to significant prolongation of plasma terminal elimination half-life (Johsnon F K, Mudd P N Jr, DiMino T, Vosk J, Sitaraman S, Boudes P, et al. An Open-label Study to Determine the Pharmacokinetics and Safety of Migalastat HCl in Subjects with Impaired Renal Function and Healthy Subjects with Normal Renal Function, Clin. Pharmacol. Drug. Dev. 2015, 4(4):256-61, which is incorporated herein by reference in its entirety). Therefore, dose adjustment, primarily by prolonged dosing intervals, will likely be required for FD renal impairment with or without ESRD.

This study also confirmed there was no clinically relevant difference in PK, extraction ratio, and dialysate clearance between standard HD and HDF and that the 123 mg dose of migalastat was well tolerated, demonstrating a similar safety profile to that observed in other trials in patients with FD who received 150 mg migalastat HCl QOD (Germain D P, Hughes D A, Nicholls K, Bichet D G, Giugliani R, Wilcox W R, et al., Treatment of Fabry's Disease with the Pharmacologic Chaperone Migalastat, N. Engl. J. Med. 2016, 375(6):545-55 and Hughes D A, Nicholls K, Shankar S P, Sunder-Plassmann G, Koeller D, Need K, et al., Oral Pharmacological Chaperone Migalastat Compared with Enzyme Replacement Therapy in Fabry Disease: 18-month Results from the Randomized Phase III ATTRACT Study, J. Med. Genet. 2017, 54(4):288-96, both of which are incorporated herein by reference in their entireties). Mild treatment emergent adverse effects (TEAEs) such as headache were the most common, and no adverse events linked to migalastat led to the discontinuation of treatment. The lack of new safety signals was a further indication that dialysis was able to clear migalastat in subjects with ESRD. Based on similar dialysis extraction ratio values and recovery of the dose in dialysate with standard HD and HDF, subjects with ESRD could receive the same dose of migalastat regardless of the dialysis method.

In order to develop the best dosing regimen in FD patients with ESRD, one must understand the mechanism of action of migalastat. In FD patients with NRF, migalastat is administered QOD. The rationale for QOD dosing is that after migalastat binds to the α-Gal A active site, migalastat improves folding, stability, and lysosomal trafficking of numerous mutant forms, as well as wild-type, of α-Gal A. If the mutation is amenable, the enzyme is trafficked to the lysosome. Migalastat quickly dissociates and clears from tissues within 24 to 48 hours post-dose, allowing the enzyme to initiate catalysis of GL-3. To allow for time intervals of intracellular trafficking, it is important to enable a dosing regimen of migalastat in ESRD that will lead a high Cₘₐₓ and C_{avg}, but a trough level that is near BQL to enable intracellular trafficking followed by catalysis of substrate. The study was designed to observe the effect of dosing migalastat 24 hours before dialysis relative to dialysis treatment being administered immediately following dosing so that the PK could be compared between timing of dialysis. As expected, plasma migalastat concentrations were considerably higher when dialysis was initiated 24 hours post-dose than when initiated immediately after dosing. Since the mechanism of action of migalastat is a concentration-driven process for intracellular trafficking, the study confirmed the timing of migalastat administration 24 hours before initiating dialysis.

Therefore, simulations were conducted using a popPK model with an adjusted dose of 82 mg QW or 123 mg QOW. The simulations derived from a popPK model showed that 82 mg QW or 123 mg QOW could theoretically be appropriate for patients with Fabry Disease and ESRD. In order to differentiate these regimens and understand the tissue distribution, these two regimens were simulated based on an updated PBPK model. This PBPK model suggested that a migalastat dose of 123 mg QOW may better balance the need for a high Cₘₐₓ above 10 μM (the concentration hypothesized to facilitate effective binding and trafficking for most α-Gal A mutants to the lysosome) with a C_{trough} that is BLQ (hypothesized to allow for migalastat dissociation from α-Gal A and systemic clearance), as compared to 82 mg QW, which is consistent with migalastat's mechanism of action. In the ESRD group receiving 123 mg QOW, the time above EC₅₀ in FD target tissues was similar to that of subjects with NRF receiving 123 mg QOD.

The fact that migalastat 123 mg QOW results in less than two-fold higher Cₘₐₓ than in subjects with NRF is not expected to lead to any safety issues given the known safety profile of migalastat. The dose suggested by the results of this PK study, along with popPK and PBPK modeling and simulations, is being studied in patients with FD and ESRD on HD or HDF in an ongoing clinical study (AT1001-025; NCT04020055) to determine potential effects of treatment of patients with FD with ESRD and amenable α-Gal A variants. Since migalastat is not currently recommended in patients with eGFR <30 mL/min/1.73 m², the results of this study could help inform the expansion of treatment options for patients with FD with severely impaired renal function who are receiving dialysis therapy.

One question is the possibility of carry-over into period 2, when subjects initiated dialysis immediately after dosing. However, carry-over of migalastat concentrations did not impact the overall results of the study, especially since all the data were included in the popPK and PBPK models (which can account for any changes in migalastat PK due to ESRD and dialysis).

Migalastat was highly extracted by dialysis in non-FD subjects with ESRD, with no new safety signals detected in this study population. PopPK and PBPK predicted that migalastat 123 mg administered QOW would be optimal in FD patients with amenable mutations and ESRD. This dose regimen is currently being studied in patients with FD and ESRD on HD or HDF (AT1001-025; NCT04020055). Since migalastat is not currently recommended in patients with eGFR <30 ml/min/1.73 m², the results of this study could help inform the expansion of treatment options for patients with FD with severely impaired renal function who are receiving dialysis therapy.

What is claimed is:

1. A method for the treatment of Fabry disease in a patient having severe renal impairment, the method comprising administering to the patient about 50 mg to about 200 mg free base equivalent (FBE) of migalastat or salt thereof at a frequency of less than once every week.

2. The method of claim 1, wherein the frequency is about once every two weeks.

3. The method of claim 1 or 2, wherein the patient is administered about 150 mg of migalastat HCl.

4. The method of any one of claims 1-3, wherein the patient is administered about 123 mg free base equivalent (FBE) of migalastat.

5. The method of any one of claims 1-4, wherein the severe renal impairment is end stage renal disease (ESRD).

6. The method of any one of claims 1-5, wherein the patient has an estimated glomerular filtration rate (eGFR) of less than about 30 mL/min/1.73 m².

7. The method of any one of claims 1-6, wherein the patient has an estimated glomerular filtration rate (eGFR) of less than about 15 ml/min/1.73 m².

8. The method of any one of claims 1-7, wherein the patient is undergoing a dialysis treatment.

9. The method of claim 8, wherein the dialysis treatment comprises hemodialysis or hemodiafiltration.

10. The method of claim 8 or 9, wherein the migalastat is administered between about 12 hours and about 36 hours prior to the dialysis treatment.

11. The method of claim 10, wherein the migalastat is administered about 24 hours prior to the dialysis treatment.

12. The method of any one of claims 8-11, wherein the dialysis treatment is performed twice per week or three times per week.

13. The method of any one of claims 1-12, wherein the patient has a HEK assay amenable mutation in α-galactosidase A.

14. The method of any one of claims 1-13, wherein the migalastat is in a solid dosage form.

15. The method of any one of claims 1-14, wherein the migalastat is administered orally.

16. A method for the treatment of Fabry disease in a patient having severe renal impairment, the method comprising administering to the patient about 50 mg to about 100 mg free base equivalent (FBE) of migalastat or salt thereof at a frequency of between about once every three days and about once every two weeks.

17. The method of claim 16, wherein the frequency is about once every seven days.

18. The method of claim 16 or 17, wherein the patient is administered about 100 mg of migalastat HCl.

19. The method of any one of claims 16-18, wherein the patient is administered about 82 mg free base equivalent (FBE) of migalastat.

20. The method of any one of claims 16-19, wherein the severe renal impairment is end stage renal disease (ESRD).

21. The method of any one of claims 16-20, wherein the patient has an estimated glomerular filtration rate (eGFR) of less than about 30 mL/min/1.73 m².

22. The method of any one of claims 16-21, wherein the patient has an estimated glomerular filtration rate (eGFR) of less than about 15 mL/min/1.73 m².

23. The method of any one of claims 16-22, wherein the patient is undergoing a dialysis treatment.

24. The method of claim 23, wherein the dialysis treatment comprises hemodialysis or hemodiafiltration.

25. The method of claim 23 or 24, wherein the migalastat is administered between about 12 hours and about 36 hours prior to the dialysis treatment.

26. The method of claim 25, wherein the migalastat is administered about 24 hours prior to the dialysis treatment.

27. The method of any one of claims 23-26, wherein the dialysis treatment is performed twice per week or three times per week.

28. The method of any one of claims 16-27, wherein the patient has a HEK assay amenable mutation in α-galactosidase A.

29. The method of any one of claims 16-28, wherein the migalastat is in a solid dosage form.

30. The method of any one of claims 16-29, wherein the migalastat is administered orally.

31. Use of migalastat in the treatment of Fabry disease in a patient having severe renal impairment, the use of migalastat comprising administering to the patient about 50 mg to about 300 mg free base equivalent (FBE) of migalastat or salt thereof at a frequency of less than once every week.

32. Use of migalastat in the treatment of Fabry disease in a patient having severe renal impairment, the use of migalastat comprising administering to the patient about 50 mg to about 100 mg free base equivalent (FBE) of migalastat or salt thereof at a frequency of between about once every three days and about once every two weeks.
